SEQUENCE LISTING (53 sequences)

SEQ ID NO: 1 - protein, 398 aa, Streptococcus pyogenes
  DOMAIN (1)..(17): Portion that was not expressed on the basis that it appeared to be a leader sequence or a transmembrane domain
MKKRILSAVLVSGVTLGAATTVGAEDLSTKIAKQDSIISNLTTEQKAAQNQVSALQAQVSSLQSEQDKLTARNTELEALSKRFEQEIKALTSQIVARNEKLKNQARSAYKNNETSGYINALLNSKSISDVVNRLVAINRAVSANAKLLEQQKADKVSLEEKQAANQTAINTIAANMAMAEENQNTLRTQQANLVAATANLALQLASATEDKANLVAQKEAAEKAAAEALAQEQAAKVKAQEQAAQQAASVEAAKSAITPAPQATPAAQSSNAIEPAALTAPAAPSAGPQTSYDSSNTYPVGQCTWGAKSLAPWAGNNWGNGGQWAYSAQAAGYRTGSTPMVGAIAVWNDGGYGHVAVVVEVQSASSIRVMESNYSGRQYIADHRGWFNPTGVTFIYPH

SEQ ID NO: 2 - protein, 374 aa, Streptococcus pyogenes
  DOMAIN (1)..(24): Portion that was not expressed on the basis that it appeared to be a leader sequence or a transmembrane domain
MKKFHRFLVSGVILLGFNGLVPTMPSTLISQQENLVHAAVLGDNYPSKWKKGNGIDSWNMYIRQCTSFAAFRLSSANGFQLPKGYGNACTWGHIAKNQGYPVNKTPSIGAIAWFDKNAYQSNAAYGHVAWVADIRGDTVTIEEYNYNAGQGPERYHKRQIPKSQVSGYIHFKDLSSQTSHSYPRQLKHISQASFDPSGTYHFTTRLPVKGQTSIDSPDLAYYEAGQSVYYDKVVTAGGYTWLSYLSFSGNRRYIPIKEPAQSVVQNDNTKPSIKVGDTVTFPGVFRVDQLVNNLIVNKELAGGDPTPLNWIDPTPLDETDNQGKVLGDQILRVGEYFIVTGSYKVLKIDQPSNGIYVQIGSRGTWVNADKANKL

SEQ ID NO: 3 - protein, 292 aa, Streptococcus pyogenes
  DOMAIN (1)..(37): Portion that was not expressed on the basis that it appeared to be a leader sequence or a transmembrane domain
MIRKYDRTSTKKKSLNWIWLIIAFFMISSFIGGSSFTESLLDILPAIAIGGTGYAIFRVRSHQKRLAKAKIAKQLEDLKAKIQLADRKVRLLDTYLADHDDFQYNVLAQQLLPQLSDIKAKAITLKDQLDPQIYRRITKKANDVESDITLQLETLQIATTLNPQPLKTPSPNLINKAPELKPYYDNIQTDHQAILAKIQGADNQEELLALHDANMRRFEDILTGYLKIKEEPKNYYNAAARLEQAKQAIQQFDEDLDETLRRLNESDLKDFDISLRIMQGATQRRTTHHQKD

SEQ ID NO: 4 - protein, 571 aa, Streptococcus pyogenes
  DOMAIN (1)..(31): Portion that was not expressed on the basis that it appeared to be a leader sequence or a transmembrane domain
MSNKKTFKKYSRVAGLLTAALIIGNLVTANAESNKQNTASTETTTTNEQPKPESSELTTEKAGQKTDDMLNSNDMIKLAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAWEWWRKVIDERDVKLSKEINVNISGSTLSPYGSITYK

SEQ ID NO: 5 - protein, 439 aa, Streptococcus pyogenes
  DOMAIN (1)..(21): Portion that was not expressed on the basis that it appeared to be a leader sequence or a transmembrane domain
MNMKKLASLAMLGASVLGLAACGGKSQKEAGASKSDTAKTEITWWAFPVFTQEKAEDGVGTYEKKLIAAFEKANPEIKVKLETIDFTSGPEKITTAIEAGTAPDVLFDAPGRIIQYGKNGKLADLNDLFTEEFTKDVNNDKLIQASKAGDTAYMYPISSAPFYMALNKKMLKDAGVLDLVKEGWTTDDFEKVLKALKDKGYNPGSFFANGQGGDQGPRAFFANLYSSHITDDKVTKYTTDDANSIKAMTKISNWIKDGLMMNGSQYDGSADIQNFANGQTSFTILWAPAQPGIQAKLLEASKVDYLEIPFPSDDGKPELEYLVNGFAVFNNKDEQKVAASKTFIQFIADDKEWGPKNVVRTGAFPVRTSYGDLYKDKRMEKIAEWTKFYSPYYNTIDGFAEMRTLWFPMVQAVSNGDEKPEDALKAFTEKANKTIKKTQ

SEQ ID NO: 6 - protein, 873 aa, Streptococcus pyogenes
  DOMAIN (1)..(26): Portion that was not expressed on the basis that it appeared to be a leader sequence or a transmembrane domain
MDLEQTKPNQVKQKIALTSTIALLSASVGVSHQVKADDRASGETKASNTHDDSLPKPETIQEAKATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKKLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKKTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKKAHLQYLRDFKLNPNRLQVIRERIDNTKQDLAKTTSSLLNAQEALAALQAKQSSLEATIATTEHQLTLLKTLANEKEYRHLDEDIATVPDLQVAPPLTGVKPLSYSKIDTTPLVQEMVKETKQLLEASARLAAENTSLVAEALVGQTSEMVASNAIVSKITSSITQPSSKTSYGSGSSTTSNLISDVDESTQRALKAGVVMLAAVGLTGFRFRKESK

SEQ ID NO: 7 - protein, 298 aa, Streptococcus pyogenes
SVGVSHQVKADDRASGETKASNTHDDSLPKPETIQEAKATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKKLEASGYIGSASYNNYYKEHADQIIAKASPGNQLNQYQ

SEQ ID NO: 8 - protein, 420 aa, Streptococcus pyogenes
  DOMAIN (1)..(1): Portion that was not expressed on the basis that it appeared to be a leader sequence or a transmembrane domain
MTKEKLVAFSQAHAEPAWLQERRLAALEAIPNLELPTIERVKFHRWNLGDGTLTENESLASVPDFIAIGDNPKLVQVGTQTVLEQLPMALIDKGVVFSDFYTALEEIPEVIEAHFGQALAFDEDKLAAYHTAYFNSAAVLYVPDHLEITTPIEAIFLQDSDSDVPFNKHVLVIAGKESKFTYLERFESIGNATQKISANISVEVIAQAGSQIKFSAIDRLGPSVTTYISRRGRLEKDANIDWALAVMNEGNVIADFDSDLIGQGSQADLKVVAASSGRQVQGIDTRVTNYGQRTVGHILQHGVILERGTLTFNGIGHILKDAKGADAQQESRVLMLSDQARADANPILLIDENEVTAGHAASIGQVDPEDMYYLMSRGLDQETAERLVIRGFLGAVIAEIPIPSVRQEIIKVLDEKLLNR

SEQ ID NO: 9 - protein, 656 aa, Streptococcus pyogenes
  DOMAIN (1)..(22): Portion that was not expressed on the basis that it appeared to be a leader sequence or a transmembrane domain
MKKSKWLAAVSVAILSVSALAACGNKNASGGSEATKTYKYVFVNDPKSLDYILTNGGGTTDVITQMVDGLLENDEYGNLVPSLAKDWKVSKDGLTYTYTLRDGVSWYTADGEEYAPVTAEDFVTGLKHAVDDKSDALYVVEDSIKNLKAYQNGEVDFKEVGVKALDDKTVQYTLNKPESYWNSKTTYSVLFPVNAKFLKSKGKDFGTTDPSSILVNGAYFLSAFTSKSSMEFHKNENYWDAKNVGIESVKLTYSDGSDPGSFYKNFDKGEFSVARLYPNDPTYKSAKKNYADNITYGMLTGDIRHLTWNLNRTSFKNTKKDPAQQDAGKKALNNKDFRQAIQFAFDRASFQAQTAGQDAKTKALRNMLVPPTFVTIGESDFGSEVEKEMAKLGDEWKDVNLADAQDGFYNPEKAKAEFAKAKEALTAEGVTFPVQLDYPVDQANAATVQEAQSFKQSVEASLGKENVIVNVLETETSTHEAQGFYAETPEQQDYDIISSWWGPDYQDPRTYLDIMSPVGGGSVIQKLGIKAGQNKDVVAAAGLDTYQTLLDEAAAITDDNDARYKAYAKAQAYLTDNAVDIPVVALGGTPRVTKAVPFSGGFSWAGSKGPLAYKGMKLQDKPVTVKQYEKAKEKWMKAKAKSNAKYAEKLADHVEK

SEQ ID NO: 10 - protein, 1647 aa, Streptococcus pyogenes
  DOMAIN (1)..(33): Portion that was not expressed on the basis that it appeared to be a leader sequence or a transmembrane domain
MEKKQRFSLRKYKSGTFSVLIGSVFLVMTTTVAADELSTMSEPTITNHAQQQAQHLTNTELSSAESKSQDTSQITLKTNREKEQSQDLVSEPTTTELADTDAASMANTGSDATQKSASLPPVNTDVHDWVKTKGAWDKGYKGQGKVVAVIDTGIDPAHQSMRISDVSTAKVKSKEDMLARQKAAGINYGSWINDKVVFAHNYVENSDNIKENQFEDFDEDWENFEFDAEAEPKAIKKHKIYRPQSTQAPKETVIKTEETDGSHDIDWTQTDDDTKYESHGMHVTGIVAGNSKEAAATGERFLGIAPEAQVMFMRVFANDIMGSAESLFIKAIEDAVALGADVINLSLGTANGAQLSGSKPLMEAIEKAKKAGVSVVVAAGNERVYGSDHDDPLATNPDYGLVGSPSTGRTPTSVAAINSKWVIQRLMTVKELENRADLNHGKAIYSESVDFKDIKDSLGYDKSHQFAYVKESTDAGYNAQDVKGKIALIERDPNKTYDEMIALAKKHGALGVLIFNNKPGQSNRSMRLTANGMGIPSAFISHEFGKAMSQLNGNGTGSLEFDSVVSKAPSQKGNEMNHFSNWGLTSDGYLKPDITAPGGDIYSTYNDNHYGSQTGTSMASPQIAGASLLVKQYLEKTQPNLPKEKIADIVKNLLMSNAQIHVNPETKTTTSPRQQGAGLLNIDGAVTSGLYVTGKDNYGSISLGNITDTMTFDVTVHNLSNKDKTLRYDTELLTDHVDPQKGRFTLTSHSLKTYQGGEVTVPANGKVTVRVTMDVSQFTKELTKQMPNGYYLEGFVRFRDSQDDQLNRVNIPFVGFKGQFENLAVAEESIYRLKSQGKTGFYFDESGPKDDIYVGKHFTGLVTLGSETNVSTKTISDNGLHTLGTFKNADGKFILEKNAQGNPVLAISPNGDNNQDFAAFKGVFLRKYQGLKASVYHASDKEHKNPLWVSPESFKGDKNFNSDIRFAKSTTLLGTAFSGKSLTGAELPDGHYHYVVSYYPDVVGAKRQEMTFDMILDRQKPVLSQATFDPETNRFKPEPLKDRGLAGVRKDSVFYLERKDNKPYTVTINDSYKYVSVEDNKTFVERQADGSFILPLDKAKLGDFYYMVEDFAGNVAIAKLGDHLPQTLGKTPIKLKLTDGNYQTKETLKDNLEMTQSDTGLVTNQAQLAVVHRNQPQSQLTKMNQDFFISPNEDGNKDFVAFKGLKNNVYNDLTVNVYAKDDHQKQTPIWSSQAGASVSAIESTAWYGITARGSKVMPGDYQYVVTYRDEHGKEHQKQYTISVNDKKPMITQGRFDTINGVDHFTPDKTKALDSSGIVREEVFYLAKKNGRKFDVTEGKDGITVSDNKVYIPKNPDGSYTISKRDGVTLSDYYYLVEDRAGNVSFATLRDLKAVGKDKAVVNFGLDLPVPEDKQIVNFTYLVRDADGKPIENLEYYNNSGNSLILPYGKYTVELLTYDTNAAKLESDKIVSFTLSADNNFQQVTFKITMLATSQITAHFDHLLPEGSRVSLKTAQDQLIPLEQSLYVPKAYGKTVQEGTYEVVVSLPKGYRIEGNTKVNTLPNEVHELSLRLVKVGDASDSTGDHKVMSKNNSQALTASATPTKSTTSATAKALPSTGEKMGLKLRIVGLVLLGLTCVFSRKKSTKD

SEQ ID NO: 11 - protein, 865 aa, Streptococcus pyogenes
ADELSTMSEPTITNHAQQQAQHLTNTELSSAESKSQDTSQITLKTNREKEQSQDLVSEPTTTELADTDAASMANTGSDATQKSASLPPVNTDVHDWVKTKGAWDKGYKGQGKVVAVIDTGIDPAHQSMRISDVSTAKVKSKEDMLARQKAAGINYGSWINDKVVFAHNYVENSDNIKENQFEDFDEDWENFEFDAEAEPKAIKKHKIYRPQSTQAPKETVIKTEETDGSHDIDWTQTDDDTKYESHGMHVTGIVAGNSKEAAATGERFLGIAPEAQVMFMRVFANDIMGSAESLFIKAIEDAVALGADVINLSLGTANGAQLSGSKPLMEAIEKAKKAGVSVVVAAGNERVYGSDHDDPLATNPDYGLVGSPSTGRTPTSVAAINSKWVIQRLMTVKELENRADLNHGKAIYSESVDFKDIKDSLGYDKSHQFAYVKESTDAGYNAQDVKGKIALIERDPNKTYDEMIALAKKHGALGVLIFNNKPGQSNRSMRLTANGMGIPSAFISHEFGKAMSQLNGNGTGSLEFDSVVSKAPSQKGNEMNHFSNWGLTSDGYLKPDITAPGGDIYSTYNDNHYGSQTGTSMASPQIAGASLLVKQYLEKTQPNLPKEKIADIVKNLLMSNAQIHVNPETKTTTSPRQQGAGLLNIDGAVTSGLYVTGKDNYGSISLGNITDTMTFDVTVHNLSNKDKTLRYDTELLTDHVDPQKGRFTLTSHSLKTYQGGEVTVPANGKVTVRVTMDVSQFTKELTKQMPNGYYLEGFVRFRDSQDDQLNRVNIPFVGFKGQFENLAVAEESIYRLKSQGKTGFYFDESGPKDDIYVGKHFTGLVTLGSETNVSTKTISDNGLHTLGTFKNADGKFILEKN

SEQ ID NO: 12 - protein, 232 aa, Streptococcus pyogenes
  DOMAIN (1)..(14): Portion that was not expressed on the basis that it appeared to be a leader sequence or a transmembrane domain
MKRIIKTIILVIIIFHGYGSVKSDSENIKDVKLQLNYAYEIIPVDYTNCNIDYLTTHDFYIDISSYKKKNFSVDSEVESYITTKFTKNQKVNIFGLPYIFTRYDVYYIYGGVTPSVNSNSENSKIVGNLLIDGVQQKTLINPIKIDKPIFTIQEFDFKIRQYLMQTYKIYDPNSPYIKGQLEIAINGNKHESFNLYDATSSSTRSDIFKKYKDNKTINMKDFSHFDIYLWTK

SEQ ID NO: 13 - protein, 319 aa, Streptococcus pyogenes
  DOMAIN (1)..(40): Portion that was not expressed on the basis that it appeared to be a leader sequence or a transmembrane domain
MKLAVLGTGMIVKEVLPVLQKIDGIDLVAILSTVRSLTTAKDLAKAHHMPLATSKYEAILGNEEIDTVYIGLPNHLHFAYAKEALLAGKHVICEKPFTMTAGELDELVVIARKRKLILLEAITNQYLSNMTFIKEHLDQLGDIKIVECNYSQYSSRYDAFKRGDIAPAFNPKMGGGALRDLNIYNIHFVVGLFGRPKTVQYLANVEKGIDTSGMLVMDYEQFKVVCIGAKDCTAEIKSTIQGNKGSLAVLGATNTLPQVQLSLHGHEPQVINHNKHDHRMYEEFVAFRDMIDQRDFEKVNQALEHSRAVMAVLERAVHS

SEQ ID NO: 14 - protein, 268 aa, Streptococcus pyogenes
  DOMAIN (1)..(19): Portion that was not expressed on the basis that it appeared to be a leader sequence or a transmembrane domain
MKKLLSLSLVAISLLNLSACESVDRAIKGDKYIDEKTAKEESEAASKAYEESIQKALKADASQFPQLTKEVGKEEAKVVMRTSQGDITLKLFPKYAPLAVENFLTHAKKGYYDNLTFHRVINDFMIQSGDPKGDGTGGESIWKGKDPKKDAGNGFVNEISPFLYHIRGALAMANAGANTNGSQFYINQNKKNQSKGLSSTNYPKPIISAYEHGGNPSLDGGYTVFGQVIDGMDVVDKIAATSINQNDKPEQDITITSIDIVKDYRFKN

SEQ ID NO: 15 - protein, 515 aa, Streptococcus pyogenes
  DOMAIN (1)..(17): Portion that was not expressed on the basis that it appeared to be a leader sequence or a transmembrane domain
MKKKILLMMSLISVFFAWQLTQAKQVLAEGKVKVVTTFYPVYEFTKGVIGNDGDVFMLMKAGTEPHDFEPSTKDIKKIQDADAFVYMDDNMETWVSDVKKSLTSKKVTIVKGTGNMLLVAGAGHDHPHEDADKKHEHNKHSEEGHNHAFDPHVWLSPYRSITVVENIRDSLSKAYPEKAENFKANAATYIEKLKELDKDYTAALSDAKQKSFVTQHAAFGYMALDYGLNQISINGVTPDAEPSAKRIATLSKYVKKYGIKYIYFEENASSKVAKTLAKEAGVKAAVLSPLEGLTEKEMKAGQDYFTVMRKNLETLRLTTDVAGKEILPEKDTTKTVYNGYFKDKEVKDRQLSDWSGSWQSVYPYLQDGTLDQVWDYKAKKSKGKMTAAEYKDYYTTGYKTDVEQIKINGKKKTMTFVRNGEKKTFTYTYAGKEILTYPKGNRGVRFMFEAKEADAGEFKYVQFSDHAIAPEKAKHFHLYWGGDSQEKLHKELEHWPTYYGSDLSGREIAQEINAH

SEQ ID NO: 16 - protein, 2045 aa, Streptococcus pyogenes
  DOMAIN (1)..(38): Portion that was not expressed on the basis that it appeared to be a leader sequence or a transmembrane domain
MRKVKKVFVSSCMLLTVGLGVAVPTGFSQSNGVMVVKAAEVPATDLSRQASDSERVDESSLLQKENLSVDSFKLENLNGWEAENDTAGNLGKFKDPDSSGYQNILTSSGKNISVAVAPKGSGKMNIKVTKRSNFQGGYYVGGLRTQTPVLKLNDVYRYSFTTKKLSGNSSEFKTRVKPVESNNKLGKELVIRVDNKNVSTKHDWLPDISDGTHTVDFTGLDKKLSVAFRFSPRQTSNVVYEFSNINIKNISPASVPAIPSKVLEGTSVLSGTAISSGDTLEKRKSFDGDILRVYKDSKIIARTVIKGNKWDVKLSKPLIAGEKLDFEILHPRSQNVSKKISKQVEAKPFDPASYKEKVIAKLKPVYEATSEKITNDAWLDENAKDLQKQKLEEQYISGKVAISEAGTKQEAIDAAYNKYSSQTDPDSLPSQYKQGNKENEQEKGRQDLIQTRDLTLKAIQEDKWLTEQEKTIQKEEALKAFETGIESVNQTVSLEQLKQRLIVYKASEKDSEKKEYPESIPNQHIPGKEKEVKAAKQEELKKLHDTTLEKINQDKWLTPDQQAEQLKQAEVTFKKGQEAIKSAQTLTQLETDLADYVSENEGKGNSIPDKYKSGNKDDLVNKAEVKLKEAHEATKQAIEKDPWLSPEQKKAQKEKAKARLDEGLKALKAADSLEILKVTEEAFVDKEKNPDSIPNQHKAGTADQARKQALDSLDKEVQKELESIDNDNTLTTDEKAAAKKKVNDAYDVAKQTAMEANSYEDLTTIKDEFLSNLPHKQGTPLKDQQSDAIAELEKKQQEIEKAIEGDKTLPRDEKEKQIADSKERLKSDTQKVKDAKNADAIKKAFEEGKVNIPQAHIPGDLNKDKEKLLAELKQKADDTEKAIDVDKTLTEDEKKEQKVKTKAELEKAKTDVKNTQTREELDKKVPELKKAIEDTHVKGNLEGVKNKAIEDLKKAHTETVAKINGDDTLDKATKEAQVKEADKALAAGKDAITKADDADKVSTAVTEHTPKIKAAHKTGDLKKAQVDANTALDKAAEKERGEINKDATLTTEDKAKQLKEVETALTKAKDNVKAAKTADAINDARDKGVATIDAVHKAGQDLGARKSGQVAKLEEAAKATKDKISADPTLTSKEKEEQSKAVDAELKKAIEAVNAADTADKVDDALGEGVTDIKNQHKSGDSIDARREAHGKELDRVAQETKGAIEKDPTLTTEEKAKQVKDVDAAKERGMAKLNEAKDADALDKAYGEGVTDIKNQHKSGDPVDARRGLHNKSIDEVAQATKDAITADTTLTEAEKETQRGNVDKEATKAKEELAKAKDADALDKAYGDGVTSIKNQHKSGKGLDVRKDEHKKALEAVAKRVTAEIEADPTLTPEVREQQKAEVQKELELATDKIAEAKDADEADKAYGDGVTAIENAHVIGKGIEARKDLAKKDLAEAAAKTKALIIEDKTLTDDQRKEQLLGVDTEYAKGIENIDAAKDAAGVDKAYSDGVRDILAQYKEGQNLNDRRNAAKEFLLKEADKVTKLINDDPTLTHDQKVDQINKVEQAKLDAIKSVDDAQTADAINDALGKGIENINNQYQHGDGVDVRKATAKGDLEKEAAKVKALIAKDPTLTQADKDKQTAAVDAAKNTAIAAVDKATTTEGINQELGKGITAINKAYRPGEGVKARKEAAKADLEKEAAKVKALITNDPTLTKADKAKQTEAVAKALKAAIAAVDKATTAEGINQELGKGITAINKAYRPGEGVKARKEAAKADLEREAAKVREAIANDPTLTKADKAKQTEAVAKALKAAIAAVDKATTAEGINQELGKGITAINKAYRPGEGVEAHKEAAKANLEKVAKETKALISGDRYLSETEKAVQKQAVEQALAKALGQVEAAKTVEAVKLAENLGTVAIRSAYVAGLAKDTDQATAALNEAKQAAIEALKQAAAETLAKITTDAKLTEAQKAEQSENVSLALKTAIATVRSAQSIASVKEAKDKGITAIRAAYVPNKAVAKSSSANHLPKSGDANSIVLVGLGVMSLLLGMVLYSKKKESKD

SEQ ID NO: 17 - protein, 805 aa, Streptococcus pyogenes
  DOMAIN (1)..(25): Portion that was not expressed on the basis that it appeared to be a leader sequence or a transmembrane domain
MNTYFCTHHKQLLLYSNLFLSFAMMGQGTAIYADTLTSNSEPNNTYFQTQTLTTTDSEKKVVQPQQKDYYTELLDQWNSIIAGNDAYDKTNPDMVTFHNKAEKDAQNIIKSYQGPDHENRTYLWEHAKDYSASANITKTYRNIEKIAKQITNPESCYYQDSKAIAIVKDGMAFMYEHAYNLDRENHQTTGKENKENWWVYEIGTPRAINNTLSLMYPYFTQEEILKYTAPIEKFVPDPTRFRVRAANFSPFEANSGNLIDMGRVKLISGILRKDDLEISDTIKAIEKVFTLVDEGNGFYQDGSLIDHVVTNAQSPLYKKGIAYTGAYGNVLIDGLSQLIPIIQKTKSPIKADKMATIYHWINHSFFPIIVRGEMMDMTRGRSISRFNAQSHVAGIEALRAILRIADMSEEPHRLALKTRIKTLVTQGNAFYNVYDNLKTYHDIKLMKELLSDTSVPVQKLDSYVASFNSMDKLALYNNKHDFAFGLSMFSNRTQNYEAMNNENLHGWFTSDGMFYLYNNDLGHYSENYWATVNPYRLPGTTETEQKPLEGTPENIKTNYQQVGMTGLSDDAFVASKKLNNTSALAAMTFTNWNKSLTLNKGWFILGNKIIFVGSNIKNQSSHKAYTTIEQRKENQKYPYCSYVNNQPVDLNNQLVDFTNTKSIFLESDDPAQNIGYYFFKPTTLSISKALQTGKWQNIKADDKSPEAIKEVSNTFITIMQNHTQDGDRYAYMMLPNMTRQEFETYISKLDIDLLENNDKLAAVYDHDSQQMHVIHYGKKATMFSNHNLSHQGFYSFPHPVRQNQQ

SEQ ID NO: 18 - protein, 318 aa, Streptococcus pyogenes
  DOMAIN (1)..(28): Portion that was not expressed on the basis that it appeared to be a leader sequence or a transmembrane domain
MKRFLNSRPWLGMVSVFFAILLFLTAASSNHNNSSSQIYSPIETYTHSLKDVPIDMKYDSDKYFISGYSYGAEVYLTSTNRIKLDSEVNNDTRNFKIVADLTHSHPGTVSVNLRVENLPSGVTATVSPDKISVTIGKKESKVFPVRGSVDAKQIANGYEISKIETGVNKVEVTSDESTIALIDHVVAKLPDDQVLDRNYSSRVTLQAVSADGTILASAIDPAKTNLSVAVKKITKSVPIRVEAVGMMDDSLSDIQYKLSKQTAVISGSREVLEDIDEIIAEVNISDVTKNTSKTVSLSSSQVSIEPSVVTVQLTTTKK

SEQ ID NO: 19 - protein, 293 aa, Streptococcus pyogenes
  DOMAIN (1)..(9): Portion that was not expressed on the basis that it appeared to be a leader sequence or a transmembrane domain
MLTFGGASAVKAEENEKVREQEKLIQQLSEKLVEINDLQTLNGDKESIQSLVDYLTRRGKLEEEWMEYLNSGIQRKLFVGPKGPAGEKGEQGPTGKQGERGETGPAGPRGDKGETGDKGAQGPVGPAGKDGQNGKDGLPGKDGKDGQNGKDGLPGKDGKDGQDGKDGLPGKDGKDGQNGKDGLPGKDGQPGKPAPKTPEVPQNPDTAPHTPKTPRIPGQSKDVTPAPQNPSNRGLNKPQTQGGNQLAKTPAAHDTHRQLPATGETTNPFFTAAAVAIMTTAGVVAVAKRQENN

SEQ ID NO: 20 - protein, 792 aa, Streptococcus pyogenes
  DOMAIN (1)..(22): Portion that was not expressed on the basis that it appeared to be a leader sequence or a transmembrane domain
MKTKKVIILVGLLLSSQLTLIACQSRGNGTYPIKTKQSRKGMTSNKIKPIKKSKKTNKTHKGVAGVDFPTDDGFILTKDSKILSKTDQGIVVDHDGHSHFIFYADLKGSPFEYLIPKGASLAKPAVAQRAASQGTSKVADPHHHYEFNPADIVAEDALGYTVRHDDHFHYILKSSLSGQTQAQAKQVATRLPQTSSLVSTATANGIPGLHFPTSDGFQFNGQGIVGVTKDSILVDHDGHLHPISFADLRQGGWAHVADQYDPAKKAEKPAETHQTPELSEREKEYQEKLAYLAEKLGIDPSTIKRVETQDGKLGLEYPHHDHAHVLMLSDIEIGKDIPDPHAIEHARELEKHKVGMDTLRALGFDEEVILDIVRTHDAPTPFPSNEKDPNMMKEWLATVIKLDLGSRKDPLQRKGLSLLPNLETLGIGFTPIKDISPVLQFKKLKQLLMTKTGVTDYRFLDNMPQLEGIDISQNNLKDISFLSKYKNLTLVAAADNGIEDIRPLGQLPNLKFLVLSNNKISDLSPLASLHQLQELHIDNNQITDLSPVSHKESLTVVDLSRNADVDLATLQAPKLETLMVNDTKVSHLDFLKNNPNLSSLSINRAQLQSLEGIEASSVIVRVEAEGNQIKSLVLKDKQGSLTFLDVTGNQLTSLEGVNNFTALDILSVSKNQLTNVNLSKPNKTVTNIDISHNNISLADLKLNEQHIPEAIAKNFPAVYEGSMVGNGTAEEKAAMATKAKESAQEASESHDYNHNHTYEDEEGHAHEHRDKDDHDHEHEDENEAKDEQNHAD

SEQ ID NO: 21 - protein, 351 aa, Streptococcus pyogenes
  DOMAIN (1)..(22): Portion that was not expressed on the basis that it appeared to be a leader sequence or a transmembrane domain
MKNSNKLIASVVTLASVMALAACQSTNDNTKVISMKGDTISVSDFYNETKNTEVSQKAMLNLVISRVFEAQYGDKVSKKEVEKAYHKTAEQYGASFSAALAQSSLTPETFKRQIRSSKLVEYAVKEAAKKELTTQEYKKAYESYTPTMAVEMITLDNEETAKSVLEELKAEGADFTAIAKEKTTTPEKKVTYKFDSGATNVPTDVVKAASSLNEGGISDVISVLDPTSYQKKFYIVKVTKKAEKKSDWQEYKKRLKAIIIAEKSKDMNFQNKVIANALDKANVKIKDKAFANILAQYANLGQKTKAASESSTTSESSKAAEENPSESEQTQTSSAEEPTETEAQTQEPAAQ

SEQ ID NO: 22 - protein, 424 aa, Streptococcus pyogenes
  DOMAIN (1)..(33): Portion that was not expressed on the basis that it appeared to be a leader sequence or a transmembrane domain
MKIGKKIVLMFTAIVLTTVLALGVYLTSAYTFSTGELSKTFKDFSTSSNKSDAIKQTRAFSILLMGVDTGSSERASKWEGNSDSMILVTVNPKTKKTTMTSLERDTLTTLSGPKNNEMNGVEAKLNAAYAAGGAQMAIMTVQDLLNITIDNYVQINMQGLIDLVNAVGGITVTNEFDFPISIAENEPEYQATVAPGTHKINGEQALVYARMRYDDPEGDYGRQKRQREVIQKVLKKILALDSISSYRKILSAVSSNMQTNIEISSRTIPSLLGYRDALRTIKTYQLKGEDATLSDGGSYQIVTSNHLLEIQNRIRTELGLHKVNQLKTNATVYENLYGSTKSQTVNNNYDSSGQAPSYSDSHSSYANYSSGVDTGQSASTDQDSTASSHRPATPSSSSDALAADESSSSGSGSLVPPANINPQT

SEQ ID NO: 23 - protein, 294 aa, Streptococcus pyogenes
  DOMAIN (1)..(19): Portion that was not expressed on the basis that it appeared to be a leader sequence or a transmembrane domain
MIKRCKGIGLALMAFFLVACVNQHPKTAKETEQQRIVATSVAVVDICDRLNLDLVGVCDSKLYTLPKRYDAVKRVGLPMNPDIELIASLKPTWILSPNSLQEDLEPKYQKLDTEYGFLNLRSVEGMYQSIDDLGNLFQRQQEAKELRQQYQDYYRAFQAKRKGKKKPKVLILMGLPGSYLVATNQSYVGNLLDLAGGENVYQSDEKEFLSANPEDMLAKEPDLILRTAHAIPDKVKVMFDKEFAENDIWKHFTAVKEGKVYDLDNTLFGMSAKLNYPEALDTLTQLFDHVGDHP

SEQ ID NO: 24 - protein, 503 aa, Streptococcus pyogenes
  DOMAIN (1)..(22): Portion that was not expressed on the basis that it appeared to be a leader sequence or a transmembrane domain
MNKNKLLRVAMLLSLLAPTAESMTVLAQDVMLETHKATTNETSDSSSKEENNKNAAPTTSDKTDQGPLDASAETNSNSLVNADDKKRSDSSQSAIGSSDNKAEAENQVDDKSTDHSKSTDHSKPTDQPKPSPSKVDTAPASSLSKQLPEARTPIQSLSPYVSDLDLSEIDIPSVNTYAAYVEHWSGKNAYTHHLLSRRYGIKADQIDSYLKSTGIAYDSTRINGEKLLQWEKKSGLDVRAIVAIAMSESSLGTQGIATLLGANMFGYAAFDLDPTQASKFNDDSAIVKMTQDTIIKNKNSNFALQDLKAAKFSRGQLNFASDGGVYFTDTTGSGKRRAQIMEDLDKWIDDHGGTPAIPAELKVQSSASFASVPAGYKLSKSYDVLGYQASSYAWGQCTWYVYNRAKELGYQFDPFMGNGGDWKYKVGYALSKTPKVGYAISFAPGQAGADGTYGHVSIVEDVRKDGSILISESNCIGLGKISYRTFTAQQAEQLTYVIGKSKN

SEQ ID NO: 25 - protein, 284 aa, Streptococcus pyogenes
  DOMAIN (1)..(21): Portion that was not expressed on the basis that it appeared to be a leader sequence or a transmembrane domain
MKSKKVVSVISLTLSLFLVTGCAKVDNNKSVNLKPATKQTYNSYSDDQLRSRENTMSVLWYQRAAETQALYLQGYQLATDRLKEQLNKPTDKPYSIVLDIDETVLDNSPYQAKNVLEGTGFTPESWDYWVQKKEAKPVAGAKDFLQFADQNGVQIYYISDRSTTQVDATMENLQKEGIPVQGRDHLLFLEKGVKSKESRRQKVKETTNVTMLFGDNLLDFADFSKKSQEDRTALLSDLQEEFGRRFIIFPNPMYGSWEGAIYKGEKLDVLKQLEERRKSLKSFK

SEQ ID NO: 26 - protein, 440 aa, Streptococcus pyogenes
  DOMAIN (1)..(15): Portion that was not expressed on the basis that it appeared to be a leader sequence or a transmembrane domain
MKNYLSIGVIALLFALTFGTVKSVQAIAGYGWLPDRPPINNSQLVVSMAGIVEGTDKKVFINFFEIDLTSQPAHGGKTEQGLSPKSKPFATDNGAMPHKLEKADLLKAIQKQLIANVHSNDGYFEVIDFASDATITDRNGKVYFADKDGSVTLPTQPVQEFLLKGHVRVRPYKEKPVQNQAKSVDVEYTVQFTPLNPDDDFRPGLKDTKLLKTLAIGDTITSQELLAQAQSILNKTHPGYTIYERDSSIVTHDNDIFRTILPMDQEFTYHVKNREQAYEINPKTGIKEKTNNTDLVSEKYYVLKQGEKPYDPFDRSHLKLFTIKYVDVNTNELLKSEQLLTASERNLDFRDLYDPRDKAKLLYNNLDAFDIMDYTLTGKVEDNHDKNNRVVTVYMGKRPKGAKGSYHLAYDKDLYTEEERKAYSYLRDTGTPIPDNPKDK

SEQ ID NO: 27 - protein, 542 aa, Streptococcus pyogenes
  DOMAIN (1)..(22): Portion that was not expressed on the basis that it appeared to be a leader sequence or a transmembrane domain
MSKYLKYFSIITLFLTGLILVACQQQKPQTKERQRKQRPKDELVVSMGAKLPHEFDPKDRYGVHNEGNITHSTLLKRSPELDIKGELAKTYHLSEDGLTWSFDLHDDFKFSNGEPVTADDVKFTYDMLKADGKAWDLTFIKNVEVVGKNQVNIHLTEAHSTFTAQLTEIPIVPKKHYNDKYKSNPIGSGPYMVKEYKAGEQAIFVRNPYWHGKKPYFKKWTWVLLDENTALAALESGDVDMIYATPELADKKVKGTRLLDIPSNDVRGLSLPYVKKGVITDSPDGYPVGNDVTSDPAIRKALTIGLNRQKVLDTVLNGYGKPAYSIIDKTPFWNPKTAIKDNKVAKAKQLLTKAGWKEQADGSRKKGDLDAAFDLYYPTNDQLRANLAVEVAEQAKALGITIKLKASNWDEMATKSHDSALLYAGGRHHAQQFYESHHPSLAGKGWTNITFYNNPTVTKYLDKAMTSSDLDKANEYWKLAQWDGKTGASTLGDLPNVWLVSLNHTYIGDKRINVGKQGVHSHGHDWSLLTNIAEWTWDESTK

SEQ ID NO: 28 - protein, 306 aa, Streptococcus pyogenes
  DOMAIN (1)..(19): Portion that was not expressed on the basis that it appeared to be a leader sequence or a transmembrane domain
MKKGFFLMAMVVSLVMIAGCDKSANPKQPTQGMSVVTSFYPMYAMTKEVSGDLNDVRMIQSGAGIHSFEPSVNDVAAIYDADLFVYHSHTLEAWARDLDPNLKKSKVDVFEASKPLTLDRVKGLEDMEVTQGIDPATLYDPHTWTDPVLAGEEAVNIAKELGRLDPKHKDSYTKNAKAFKKEAEQLTEEYTQKFKKVRSKTFVTQHTAFSYLAKRFGLKQLGISGISPEQEPSPRQLKEIQDFVKEYNVKTIFAEDNVNPKIAHAIAKSTGAKVKTLSPLEAAPSGNKTYLENLRANLEVLYQQLK

SEQ ID NO: 29 - protein, 309 aa, Streptococcus pyogenes
  DOMAIN (1)..(22): Portion that was not expressed on the basis that it appeared to be a leader sequence or a transmembrane domain
MKQMNKLITGVVTLATVVTLSACQSSHNNTKLVSMKGDTITVSDFYNETKNTELAQKAMLSLVISRVFETQYANKVSDKEVEKAYKQTADQYGTSFKTVLAQSGLTPETYKKQIRLTKLVEYAVKEQAKNETISKKDYRQAYDAYTPTMTAEIMQFEKEEDAKAALEAVKAEGADFAAIAKEKTTAADKKTTYTFDSGETTLPAEVVRAASGLKEGNRSEIITALDPATSKRTYHIIKVTKKATKKADWKAYQKRLKDIIVTGKLKDPDFQNKVIAKALDKANVKIKDKAFANILAQFAKPNQKQPAQK

SEQ ID NO: 30 - protein, 498 aa, Streptococcus pyogenes
  DOMAIN (1)..(20): Portion that was not expressed on the basis that it appeared to be a leader sequence or a transmembrane domain
MINKKISLGVLSILTAFSLQSVSYACTGFIIGKDLTKDGSLLYGRTEDLEPHHNKNFIVRLAKDNPAGEKWKDLSNGFEYPLPEHSYRYSAIPDVTPNKGVYDEAGFNEFGVSMSATVSASANDAIQKIDPYVKNGLAESSMTSVILPSVKTAREGVALIAKIVTEKGAAEGNIVTLADKDGIWYMEILSGHQYVAIKFPDDKYAVFPNTFYLGHVDFNDKENTIASEDVEKVAKKAKSYTEVDGKFHIAKSYNPPLNDANRSRSFSGIKSLDPDSKVTYKDSNYELLQSTDKTFSLEDAMKLQRNRFEGLDLKPLDQMALDGKGKPKSKKAVKGYAYPISNPNVMEAHIFQLKKDIPAELGGVMWLSIGSPRNAPYLPYLGNISRTYEAYQEKSTQYNDKSWYWTVSHINDLVAAHPKPFGTKVIDEMKGLEKTWIAEQDKSTKEISDLVVSDPKAAQEKADKISLDRAEKTFKRLKAIEAKLVKEKPKNKKGLNRS

SEQ ID NO: 31 - protein, 910 aa, Streptococcus pyogenes
  DOMAIN (1)..(1): Portion that was not expressed on the basis that it appeared to be a leader sequence or a transmembrane domain
MINKKCIIPVSLLTLAITLTSVEEVTSRQNLTYANEIVTQRPKRESVISDKSNFPVISPYLASVDFGERKTPLPTPDKGVKVTTEQSIAQVRKGPEERPYTVTGKITSVINGWGGYGFYIQDSEGIGLYVYPQKDLGYSKGDIVQLTGTLTRFKGDLQLQQVTAHKKLELSFPTSVKEAVISELETTTPSTLVKLSHVTVGELSTDQYNNTSFLVRDDSGKSIVVHIDHRTGVKGADVVTKISQGDLINLTAILSIVDGQLQLRPFSLEQLEVVKKVTSSNSDASSRNIVKIGEIQGASHTSPLLKKAVTVEQVVVTYLDDSTHFYVQDLNGDGDLATSDGIRVFAKNAKVQVGDVLTISGEVEEFFGRGYEERKQTDLTITQIVAKAVTKTGTAQVPSPLVLGKDRIAPANIIDNDGLRVFDPEEDAIDYWESMEGMLVAVDDAKILGPMKNKEIYVLPGSSTRPLNNSGGVLLPANSYNTDVIPVLFKKGKQIIKAGDSYKGRLAGPVSYSYGNYKVFVDDSKNMPSLMDGHLKPEKTNLQKDLSKLSIASYNIENFSANPSSTKDEKVKRIAESFIHDLNAPDIIGLIEVQDNNGPTDDGTTDATQSAQRLIDAIKKLGGPTYRYVDIAPENNVDGGQPGGNIRTGFLYQPERVSLSDKPKGGARDALTWVNGELNLSVGRIDPTNAAWKDVRKSLAAEFIFQGRKVVVVANHLNSKRGDNALYGCVQPVTFKSEQRRHVLANMLAQFAKEGAKHQANIVMLGDFNDFEFTKTIQLIEEGDMVNLVSRHDISDRYSYFHQGNNQTLDNILVSRHLLDHYEFDMVHVNSPFMEAHGRASDHDPLLLQLSFSKENDKAESSKQSVKAKKTSKGKLLPKTGDSLVYVITLLGTASLLVPILLLTKGKKES

SEQ ID NO: 32 - protein, 1008 aa, Streptococcus pyogenes
  DOMAIN (1)..(21): Portion that was not expressed on the basis that it appeared to be a leader sequence or a transmembrane domain
MKKHLKTVALTLTTVSVVTHNQEVFSLVKEPILKQTQASSSISGADYAESSGKSKLKINETSGPVDDTVTDLFSDKRTTPEKIKDNLAKGPREQELKAVTENTESEKQITSGSQLEQSKESLSLNKTVPSTSNWEICDFITKGNTLVGLSKSGVEKLSQTDHLVLPSQAADGTQLIQVASFAFTPDKKTAIAEYTSRAGENGEISQLDVDGKEIINEGEVFNSYLLKKVTIPTGYKHIGQDAFVDNKNIAEVNLPESLETISDYAFAHLALKQIDLPDNLKAIGELAFFDNQITGKLSLPRQLMRLAERAFKSNHIKTIEFRGNSLKVIGEASFQDNDLSQLMLPDGLEKIESEAFTGNPGDDHYNNRVVLWTKSGKNPSGLATENTYVNPDKSLWQESPEIDYTKWLEEDFTYQKNSVTGFSNKGLQKVKRNKNLEIPKQHNGVTITEIGDNAFRNVDFQNKTLRKYDLEEVKLPSTIRKIGAFAFQSNNLKSFEASDDLEEIKEGAFMNNRIETLELKDKLVTIGDAAFHINHIYAIVLPESVQEIGRSAFRQNGANNLIFMGSKVKTLGEMAFLSNRLEHLDLSEQKQLTEIPVQAFSDNALKEVLLPASLKTIREEAFKKNHLKQLEVASALSHIAFNALDDNDGDEQFDNKVVVKTHHNSYALADGEHFIVDPDKLSSTIVDLEKILKLIEGLDYSTLRQTTQTQFRDMTTAGKALLSKSNLRQGEKQKFLQEAQFFLGRVDLDKAIAKAEKALVTKKATKNGQLLERSINKAVLAYNNSAIKKANVKRLEKELDLLTGLVEGKGPLAQATMVQGVYLLKTPLPLPEYYIGLNVYFDKSGKLIYALDMSDTIGEGQKDAYGNPILNVDEDNEGYHALAVATLADYEGLDIKTILNSKLSQLTSIRQVPTAAYHRAGIFQAIQNAAAEAEQLLPKPGTHSEKSSSSESANSKDRGLQSNPKTNRGRHSAILPRTGSKGSFVYGILGYTSVALLSLITAIKKKKY

SEQ ID NO: 33 - protein, 364 aa, Streptococcus pyogenes
  DOMAIN (1)..(1): Portion that was not expressed on the basis that it appeared to be a leader sequence or a transmembrane domain
MVDLGFSLYPERYDVTKSKAYIDLCHSYGAKRLFMSLLQLAPADHQMFHCYAELIAYANQLGIRVIADVSPSFISQAGWSDQLIERAHAFGLAGLRLDEALPLAEIVTLTRNPFGLKIELNMSTDKQLLMSLLATDAERSNIIGCHNFYPHEFTGLSWQHFKDMSRFYHEHDIETAAFITAQSASEGPWLLAEGLPTVEDHRHLPIGLQVELMKAIGTIDNILISNQFISEEELAACTQALARPVTTIKVRPIIDLTEVEEQIIGYPHCYRGDVSDYVIRSTMPRLVYAQESIAPRDQSKEVKRGSIIIDNDRYHRYKGELQIALKNFTVSSKANVVAEVREDYLSLLDDLRPWQEFCLEIAPS

SEQ ID NO: 34 - protein, 1165 aa, Streptococcus pyogenes
  DOMAIN (1)..(1): Portion that was not expressed on the basis that it appeared to be a leader sequence or a transmembrane domain
MKKKVNQGSKRYQYLLKKWGIGFVIAATGTVVLGCTPSILTHQVAAKTIVGLARDEAQQGDGNAKSGDGLQSSSKEAKPVLDSSSANPASIAEHHLRMHFKTLPAGESLGSLGLWVWGDVDQPSKDWPNGAITMTKAKKDDYGYYLDVPLAAKHRQQVSYLINNKAGENLSKDQHISLLTPKMNEVWIDENYHAHAYRPLKKGYLRINYHNQSGHYDNLAVWTFKDVKTPTTDWPNGLDLSHKGHYGAYVDVPLKEGANEIGFLILDKSKTGDAIKVQPKDYLFKELDNHTQVFVKDTDPKVYNNPYYIDQVSLKGAEQTTPNEIKAIFTTLDGLDEDAVKQNIKITDKAGKTVAIDELTLDRDKSVMTLKGDFKAQGAVYTVTFGEVSQVARQSWQLKDKLYAYDGELGATLAKDGSVDLALWSPSADTVKVVVYDKQDQTRVVGQADLTKSDKGVWRAHLTSDSVKGISDYTGYYYLYEITRGQEKVMVLDPYAKSLAAWNDATATDDIKTAKAAFIDPSKLGPTGLDFAKINNFKKREDAIIYEAHVRDFTSDKALEGKLTHPFGTFSAFVEQLDYLKDLGVTHVQLLPVLSYFYANELDKSRSTAYTSSDNNYNWGYDPQHYFALSGMYSANPNDPALRIAELKNLVNEIHKRGMGVIFDVVYNHTARTYLFEDLEPNYYHFMNADGTARESFGGGRLGTTHAMSRRILVDSITYLTREFKVDGFRFDMMGDHDAAAIEQAFKAAKAINPNTIMIGEGWRTYQGDEGKKEIAADQDWMKATNTVGVFSDDIRNTLKSGFPNEGTAAFITGGAKNLEGLFKTIKAQPGNFEADAPGDVVQYIAAHDNLTLHDVIAKSINKDPKVAEEEIHKRIRLGNTMILTAQGTAFIHSGQEYGRTKQLLNPDYKTKASDDKVPNKATLIDAVAQYPYFIHDSYDSSDAVNHFDWAKATDSIAHPISNQTKAYTQGLIALRRSTDAFTKATKAEVDRDVTLITQAGQDGIQQEDLIMGYQTVASNGDRYAVFVNADNKTRKVVLPQAYRYLLGAQVLVDAEQAGVTAIAKPKGVQFTKEGLTIEGLTALVLKVSSKTANPSQQKSQTDNHQTKTPDGSKDLDKSLMTRPKRAKTNQKLPKTGEASSKGLLAAGIALLLLAISLLMKRQKD

SEQ ID NO: 35 - protein, 348 aa, Streptococcus pyogenes
  DOMAIN (1)..(37): Portion that was not expressed on the basis that it appeared to be a leader sequence or a transmembrane domain
MLTSKHHNLNKLVWRYGLTSAAAVLLAFGGGASSVKAEVSSTTMTSSQRESKIKEIEESLKKYPEVSNEKFWERKWYGTYFKEEDFQKELKDFTEKRLKEILDLIGKSGIKGDRGETGPAGPAGPQGKTGERGAQGPKGDRGEQGIQGKAGEKGERGEKGDKGETGERGEKGEAGIQGPQGEAGKDGAPGKDGAPGEKGEKGDRGETGAQGPVGPQGEKGETGAQGPAGPQGEAGKPGEQGPAGPQGEAGQPGEKAPEKSPEGEAGQPGEKAPEKSKEVTPAAEKPADKEANQTPERRNGNMAKTPVANNHRRLPATGEQANPFFTAAAVAVMTTAGVLAVTKRKENN

SEQ ID NO: 36 - protein, 379 aa, Streptococcus pyogenes
  DOMAIN (1)..(37): Portion that was not expressed on the basis that it appeared to be a leader sequence or a transmembrane domain
MRRAENNKHSRYSIRKLSVGVTSIAIASLFLGKVAYAVDGIPPISLTQKTTATTSENWHHIDKDGLIPLGISLEAAKEEFKKEVEESRLSEAQKETYKQKIKTAPDKDKLLFTYHSEYMTAVKDLPASTESTTQPVEAPVQETQASASDSMVTGDSTSVTTDSPEETPSSESPVAPALSEAPAQPAESEEPSVAASSEETPSPSTPAAPETPEEPAAPSPSPESEEPSVAAPSEETPSPETPEEPAAPSQPAESEESSVAATTSPSPSTPAESETQTPPAVTKDSDKPSSAAEKPAASSLVSEQTVQQPTSKRSSDKKEEQEQSYSPNRSLSRQVRAHESGKYLPSTGEKAQPLFIATMTLMSLFGSLLVTKRQKETKK

SEQ ID NO: 37 - protein, 1181 aa, Streptococcus pyogenes
  DOMAIN (1)..(25): Portion that was not expressed on the basis that it appeared to be a leader sequence or a transmembrane domain
MRKKQKLPFDKLAIALMSTSILLNAQSDIKANTVTEDTPATEQAVETPQPTAVSEEAPSSKETKTPQTPDDAEETIADDANDLAPQAPAKTADTPATSKATIRDLNDPSQVKTLQEKAGKGAGTVVAVIDAGFDKNHEAWRLTDKTKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIIDAVNLGAKVINMSFGNAALAYANLPDETKKAFDYAKSKGVSIVTSAGNDSSFGGKTRLPLADHPDYGVVGTPAAADSTLTVASYSPDKQLTETATVKTADQQDKEMPVLSTNRFEPNKAYDYAYANRGMKEDDFKDVKGKIALIERGDIDFKDKIANAKKAGAVGVLIYDNQDKGFPIELPNVDQMPAAFISRKDGLLLKENPQKTITFNATPKVLPTASGTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKLFALAPKALYETSWQKITIPANSSKQVTIPIDVSQFSKDLLAPMKNGYFLEGFVRFKQDPTKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQDGSDQAPDKKPETKPEQDGSGQAPDKKPETKPEQDGSGQTPDKKPETKPEQDGSGQTPDKKPETKPEKDSSGQTPGKTPQKGQPSRTLEKRSSKRALATKASTKDQLPTTNDKDTNRLHLLKLVMTTFFLGLVAHIFKTKRTED

SEQ ID NO: 38 - protein, 484 aa, Streptococcus pyogenes
  DOMAIN (1)..(1): Portion that was not expressed on the basis that it appeared to be a leader sequence or a transmembrane domain
MAKNNTNRHYSLRKLKTGTASVAVALTVLGAGFANQTEVKANGDGNPREVIEDLAANNPAIQNIRLRYENKDLKARLENAMEVAGRDFKRAEELEKAKQALEDQRKDLETKLKELQQDYDLAKESTSWDRQRLEKELEEKKEALELAIDQASRDYHRATALEKELEEKKKALELAIDQASQDYNRANVLEKELETITREQEINRNLLGNAKLELDQLSSEKEQLTIEKAKLEEEKQISDASRQSLRRDLDASREAKKQVEKDLANLTAELDKVKEDKQISDASRQGLRRDLDASREAKKQVEKDLANLTAELDKVKEEKQISDASRQGLRRDLDASREAKKQVEKALEEANSKLAALEKLNKELEESKKLTEKEKAELQAKLEAEAKALKEQLAKQAEELAKLRAGKASDSQTPDTKPGNKAVPGKGQAPQAGTKPNQNKAPMKETKRQLPSTGETANPFFTAAALTVMATAGVAAVVKRKEEN

SEQ ID NO: 39 - protein, 541 aa, Streptococcus pyogenes
  DOMAIN (1)..(1): Portion that was not expressed on the basis that it appeared to be a leader sequence or a transmembrane domain
MKKRKLLAVTLLSTILLNSAVPLVVADTSLRNSTSSTDQPTTADTDTDDESETPKKDKKSKETASQHDTQKDHKPSHTHPTPPSNDTKQTDQASSEATDKPNKDKNDTKQPDSSDQSTPSPKDQSSQKESQNKDGRPTPSPDQQKDQTPDKTPEKSADKTPEKGPEKATDKTPEPNRDAPKPIQPPLAAAPVFIPWRESDKDLSKLKPSSRSSAAYVRHWTGDSAYTHNLLSRRYGITAEQLDGFLNSLGIHYDKERLNGKRLLEWEKLTGLDVRAIVAIAMAESSLGTQGVAKEKGANMFGYGAFDFNPNNAKKYSDEVAIRHMVEDTIIANKNQTFERQDLKAKKWSLGQLDTLIDGGVYFTDTSGSGQRRADIMTKLDQWIDDHGSTPEIPEHLKITSGTQFSEVPVGYKRSQPQNVLTYKSETYSFGQCTWYAYNRVKELGYQVDRYMGNGGDWQRKPGFVTTHKPKVGYVVSFAPGQAGADATYGHVAVVEQIKEDGSILISESNVMGLGTISYRTFTAEQASLLTYVVGDKLPRP

SEQ ID NO: 40 - protein, 271 aa, Streptococcus pyogenes
  DOMAIN (1)..(1): Portion that was not expressed on the basis that it appeared to be a leader sequence or a transmembrane domain
MNLLGSRRVFSKKCRLVKFSMVALVSATMAVTTVTLENTALARQTQVSNDVVLNDGASKYLNEALAWTFNDSPNYYKTLGTSQITPALFPKAGDILYSKLDELGRTRTARGTLTYANVEGSYGVRQSFGKNQNPAGWTGNPNHVKYKIEWLNGLSYVGDFWNRSHLIADSLGGDALRVNAVTGTRTQNVGGRDQKGGMRYTEQRAQEWLEANRDGYLYYEAAPIYNADELIPRAVVVSMQSSDNTINEKVLVYNTANGYTINYHNGTPTQK

SEQ ID NO: 41 - protein, 310 aa, Streptococcus pyogenes
  DOMAIN (1)..(21): Portion that was not expressed on the basis that it appeared to be a leader sequence or a transmembrane domain
MGKRMSLILGAFLSVFLLVACSSTGTKTAKSDKLKVVATNSIIADMTKAIAGDKIDLHSIVPIGQDPHEYEPLPEDVEKTSNADVIFYNGINLEDGGQAWFTKLVKNAQKTKNKDYFAVSDGIDVIYLEGASEKGKEDPHAWLNLENGIIYSKNIAKQLIAKDPKNKETYEKNLKAYVAKLEKLDKEAKSKFDAIAENKKLIVTSEGCFKYFSKAYGVPSAYIWEINTEEEGTPDQISSLIEKLKVIKPSALFVESSVDRRPMETVSKDSGIPIYSEIFTDSIAKKGKPGDSYYAMMKWNLDKISEGLAK

SEQ ID NO: 42 - protein, 235 aa, Streptococcus pyogenes
  DOMAIN (1)..(24): Portion that was not expressed on the basis that it appeared to be a leader sequence or a transmembrane domain
MTKKKGKLVLISLFVLAACLGAYSAMRQSHKTSNVSAETIASSSTRHFIDEIGPTASTIGQERDLYASVMIAQAILESSNGKSSLSQAPYYNFFGIKGAYNGSSVTMSTWEDDGNGNTYTIDQAFRAYPSIADSLNDYADLLSSSTYIGARKSNTLSYQDATAALTGLYATDTSYNLKLNNIIATYGLTAYDVANSSAQETGLATSGYVWNEYRRNYTDAETLAVDEAWAKRMTY

SEQ ID NO: 43 - protein, 225 aa, Streptococcus pyogenes
  DOMAIN (1)..(1): Portion that was not expressed on the basis that it appeared to be a leader sequence or a transmembrane domain
MSSVGVINLRNLYSTYDPTEVKGKINEGPPFSGSLFYKNIPYGNSSIELKVELNSVEKANFFSGKRVDIFTLEYSPPCNSNIKKNSYGGITLSDGNRIDKKNIPVNIFIDGVQQKYSYTDISTGSTDKKEVTIQELDVKSRYYLQKHFNIYGFGDVKDFGRSSRFQSGFEEGNIIFHLNSGERISYNLFDTGHGDRESMLKKYSDNKTAYSDQLHIDIYLVKFNK

SEQ ID NO: 44 - protein, 419 aa, Streptococcus pyogenes
  DOMAIN (1)..(24): Portion that was not expressed on the basis that it appeared to be a leader sequence or a transmembrane domain
MSWNWKKTSVLGTLSLASVLPLTACVSGGGKGVKETDGKTIVVSVDEGYVDYIKSIKGEFEKEHKVTVKVKKEGMMDTLDKLSTDGPTGASPDVFLAPFDRVGGLGTEGQIAEVTLGNSKEFDDTVKKLVTIDGKTYGAPDVIETLVTYYNKDLVPQAPKSFTELEVLQKDSKFAFASEPGKSVGFLAKWTDFYYGYGLIAGYGGYIFGDKGTKPSDLGLGNDGTVEGLNYAKQWYGTWPQGMQDTKKAGDFITEQFISKKAGVIIDGPWAASSFKDAGVNFGVMEIPTLTNGKKYQPFAGGKAWVISNYSKGKTTAQKFLDYVTNAENQKRFYDKTQEIPANLTARNYASKEGNELTKAVISQFESAQPMPNIPEMAEVWEPGANMFFNVASGKEEASKAAKEAAKTIKEAIEQKYAE

SEQ ID NO: 45 - protein, 265 aa, Streptococcus pyogenes
  DOMAIN (1)..(31): Portion that was not expressed on the basis that it appeared to be a leader sequence or a transmembrane domain
MTTMQKTISLLSLALLIGLLGTSGKAISVYAQDQHTDNVIAESTISQVSVEASMRGTEPYIDATVTTDQPVRQPTQATITLKDASDNTINSWVYTMAAQQRRFTAWFDLTGQKSGDYHVTVTVHTQEKAVTGQSGTVHFDQNKARKTPTNMQQKDTSKAMTNSVDVDTKAQTNQSANQEIDSTSNPFRSATNHRSTSLKRSTKNEKLTPTASNSQKNGSNKTKMLVDKEEVKPTSKRGFPWVLLGLVVSLAAGLFIAIQKVSRRK

SEQ ID NO: 46 - protein, 995 aa, Streptococcus pyogenes
  DOMAIN (1)..(25): Portion that was not expressed on the basis that it appeared to be a leader sequence or a transmembrane domain
MDKHLLVKRTLGCVCAATLMGAALATHHDSLNTVKAEEKTVQVQKGLPSIDSLHYLSENSKKEFKEELSKAGQESQKVKEILAKAQQADKQAQELAKMKIPEKIPMKPLHGSLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 47 - protein, 824 aa, Streptococcus pyogenes
  DOMAIN (1)..(393): Portion that was not expressed on the basis that it appeared to be a leader sequence or a transmembrane domain
MIKDTFLKTNWLNISHHIILLVFGFYFSFYSLAKELVSSTAQPVNYYAHLLNVSFVGYIISLIGLSYYLSRQVSRQLFLKTSFIVISYLIVSYWVQITQHLNDKRFDIWSLTKNQFYQFQALPSLLIILVMATLIKILAAYFAIEKDRFGLLGYQGNTFSVALILAVVPINDIHLLKLISSRFSELVTAGNSQIALLKISGLLIVLLVIFATIIYVVLNALKHLKSNKPSFSVAATTSLFLALVFNYTFQYGVKGDEALLGYYVFPGATLFQIVAITLVALLAYVITNRYWPTTFFLLILGTIISVVNDLKESMRSEPLLVTDFVWLQELGLVTSFVKKSVIVEMVVGLAICIVVAWYLHGRVLAGKLFMSPVKRASAVLGLFIVSCSMLIPFSYEKEGKILSGLPIISALNNDNDINWLGFSTNARYKSLAYVWTRQVTKKIMEKPTNYSQETIASIAQKYQKLAEDINKDRKNNIADQTVIYLLSESLSDPDRVSNVTVSHDVLPNIKAIKNSTTAGLMQSDSYGGGTANMEFQTLTSLPFYNFSSSVSVLYSEVFPKMAKPHTISEFYQGKNRIAMHPASANNFNRKTVYSNLGFSKFLALSGSKDKFKNIENVGLLTSDKTVYNNILSLINPSESQFFSVITMQNHIPWSSDYPEEIVAEGKNFTEEENHNLTSYARLLSFTDKETRAFLEKLTQINKPITVVFYGDHLPGLYPDSAFNKHIENKYLTDYFIWSNGTNEKKNHPLINSSDFTAALFEHTDSKVSPYYALLTEVLNKASVDKSPDSPEVKAIQNDLKNIQYDVTIGKGYLLKHKTFFKISR

SEQ ID NO: 48 - protein, 406 aa, Streptococcus pyogenes
  DOMAIN (1)..(36): Portion that was not expressed on the basis that it appeared to be a leader sequence or a transmembrane domain
MENWKFALSSIWGHKMRSILTMLGIIIGVAAVVIIMGLGNAMKNSVTSTFSSKQKDIQLYFQEKGEEEDLYAGLHTHENNHEVKPEWLEQIVKDIDGIDSYYFTNSATSTISYEKKKVDNASIIGVSKDYFNIKNYDIVAGRTLTDNDYSNFSRIILLDTVLADDLFGKGNYKSALNKVVSLSDKDYLVIGVYKTDQTPVSFDGLSGGAVMANTQVASEFGTKEIGSIYIHVNDIQNSMNLGNQAADMLTNISHIKDGQYAVPDNSKIVEEINSQFSIMTTVIGSIAAISLLVGGIGVMNIMLVSVTERTREIGLRKALGATRLKILSQFLIESVVLTVLGGLIGLLLAQLSVGALGNAMTLKGACISLDVALIAVLFSASIGVFFGMLPANKASKLDPIEALRYE

SEQ ID NO: 49 - protein, 564 aa, Streptococcus pyogenes
  DOMAIN (1)..(1): Portion that was not expressed on the basis that it appeared to be a leader sequence or a transmembrane domain
MAKNTTNRHYSLRKLKTGTASVAVALTVVGAGLVAGQTVRADHSDLVAEKQRLEDLGQKFERLKQRSELYLQQYYDNKSNGYKGDWYVQQLKMLNRDLEQAYNELSGEAHKDALGKLGIDNADLKAKITELEKSVEEKNDVLSQIKKELEEAEKDIQFGREVHAADLLRHKQEIAEKENVISKLNGELQPLKQKVDETDRNLQQEKQKVLSLEQQLAVTKENAKKDFELAALGHQLADKEYNAKIAELESKLADAKKDFELAALGHQHAHNEYQAKLAEKDGQIKQLEEQKQILDASRKGTARDLEAVRQAKKATEAELNNLKAELAKVTEQKQILDASRKGTARDLEAVRKSKKQQVEAALKQLEEQNKISEASRKGLRRDLDTSREAKKQVEKDLANLTAELDKVKEEKQISDASRQGLRRDLDASREAKKQVEKALEEANSKLAALEKLNKDLEESKKLTEKEKAELQAKLEAEAKALKEQLAKQAEELAKLRAGKASDSQTPDAKPGNKAVPGKGQAPQAGTKPNQNKAPMKETKRQLPSTGETANPFFTAAALTVMAAA

SEQ ID NO: 50 - protein, 500 aa, Streptococcus pyogenes
  DOMAIN (466)..(500): Portion that was not expressed on the basis that it appeared to be a leader sequence or a transmembrane domain
MAKNNTNRHYSLRKLKTGTASVAVALTVLGTGLASQTEVKADGEARDVVPELVANNLGLLRKRVARLQAELKTKEEKLRKLDLALGKEHIDNIALKHQLETEKREAEAQRQILENEKKKLEEELANKNTTLDGALRAITEKEEKLRELDLALGKEHIDNIDLKHQLETEKREAEAQRQILENEKKKLEEELANKNTTLDGALRAITEKEEKLRELDLALGKEHIDNIDLKHQLETEKREAEAQRQILENEKKKLEEQNKISEASRKGLRRDLDASREAKKQLEAEHQKLEEQNKISEASRKGLRRDLDASREAKKQVEKDLANLTAELDKVKEEKQISDASRQGLRRDLDASREAKKQVEKALEEANSKLAALEKLNKELEESKKLTEKEKAELQAKLEAEAKALKEQLAKQAEELAKLRAGKASGSQTPDAKPGNKAVPGKGQAPQAGAKPNQNKAPMKETKRQLPSTGETANPFLTAAALTVMATAGVAAVVKRKEEN

SEQ ID NO: 51 - protein, 337 aa, Streptococcus pyogenes
  DOMAIN (1)..(1): Portion that was not expressed on the basis that it appeared to be a leader sequence or a transmembrane domain
MARKDTNKQYSLRKLKTGTASVAVAVAVLGAGFANQTTVKANSKNPVPVKKEAKLSEAELHDKIKNLEEEKAELFEKLDKVEEEHKKVEEEHKKDHEKLEKKSEDVERHYLRQLDQEYKEQQERQKNLEELERQSQREVEKRYQEQLQKQQQLEKEKQISEASRKSLSRDLEASRAAKKDLEAEHQKLKEEKQISEASRQGLSRDLEASREAKKKVEADLAEANSKLQALEKLNKELEEGKKLSEKEKAELQAKLEAEAKALKEQLAKQAEELAKLKGNQTPNAKVAPQANRSRSAMTQQKRTLPSTGETANPFFTAAAATVMVSAGMLALKRKEEN

SEQ ID NO: 52 - protein, 581 aa, Streptococcus pyogenes
  DOMAIN (1)..(1): Portion that was not expressed on the basis that it appeared to be a leader sequence or a transmembrane domain
MAKNNTNRHYSLRKLKTGTASVAVALTVLGTGLVAGQTVKADARSVNGEFPRHVKLKNEIENLLDQVTQLYTKHNSNYQQYNAQAGRLDLRQKAEYLKGLNDWAERLLQELNGEDVKKVLGKVAFEKDDLEKEVKELKEKIDKKEKEYQDLDKDFDLAKQGYVLSDKRHQQELEEKEKKVTEATAKVGQISEELETVKQKVESTMQDLTEKQNRVSQLEQELATTKQNAKEDFELAALANAADKQKLEAKIADLETKLKEAKEDFELAALGHQHAHNEYQAKLAEKDDQIKQLEEQKQILDASRKGTARDLEAVRQAKKATEAELNNLKAELAKVTEQKQILDASRKGTARDLEAVRQAKAQVEAALKQLEEQNRISEASRKGLRRDLDASREAKKQVEKDLANLTAELDKVKEEKQISDASRQGLRRDLDASREAKKQVEKALEEANSKLAALEKLNKELEESKKLTEKEKAELQAKLEAEAKALKEQLAKQAEELAKLRAGKASDSQIPDTKPGNKAVPGKGQAPQAGTKPNQNKAPMKETKRQLPSTGETANPFFTAAALTVMATAGVAAVVKRKEEN

SEQ ID NO: 53 - protein, 384 aa, Streptococcus pyogenes
  DOMAIN (1)..(1): Portion that was not expressed on the basis that it appeared to be a leader sequence or a transmembrane domain
MARKDTNKQYSLRKLKTGTASVAVAVAVLGAGFANQTEVKAEGVKKAEEAKLSVPKTEYDKLYDDYDKLQEKSAEYLERIGELEERQKNLEKLERQSQVAADKHYQEQVKKHQEYKQEQEERQKNLEELERQNKREIDKRYKEQLHKQQQLETEKQISEASRKSLSRDLEASREAKKKVEADLAALEAEHQKLKEEKQISDASRQSLSRDLEASREAKKKVEADLAALTAEHQKLKEEKQISDASRQGLSRDLEASREAKKKVEADLAEANSKLQALEKLNKELEEGKKLSEKEKAELQAKLEAEAKALKEQLAKQAEELAKLKGNQTPNAKVAPQANRSRSAMTQQKRTLPSTGETANPFFTAAAATVMVSAGMLALKRKEEN

(19) United States
(12) Patent Application Publication
(10) Pub. No.: US 2012/0088685 A1
(43) Pub. Date: Apr. 12, 2012

(54) DIAGNOSIS OF NEUROPSYCHIATRIC AND BEHAVIOURAL DISORDERS

(76) Inventors: Graziella Orefici, Roma (IT); Guido Grandi, Segrate (IT); Immaculada Margarity Ros, Siena (IT); Renata Marie Grifantini, Siena (IT); Giuliano Bensi, Siena (IT); Mauro Bombaci, Siena (IT)

(21) Appl. No.: 13/254,636

(22) PCT Filed: Mar. 5, 2010
(86) PCT No.: PCT/IB10/50967

§ 371 Date: Dec. 20, 2011

(30) Foreign Application Priority Data
Mar. 5, 2009 (GB) 0903858.9

(51) Int. Cl. C40B 30/04; C40B 40/10; G01N 33/569

(52) U.S. Cl. 506/9; 436/501; 506/18

(57) ABSTRACT

This invention provides a method of identifying GAS infection as the cause of a neuropsychiatric or behavioural disorder in a patient, or of identifying a patient at risk of developing a neuropsychiatric or behavioural disorder caused by GAS infection. This invention also provides associated protein arrays.

### TECHNICAL FIELD

This invention is in the field of identifying patients having neuropsychiatric and behavioural disorders associated with Streptococcus pyogenes (Group A Streptococcus; GAS) infection and identifying patients at risk of developing neuropsychiatric and behavioural disorders associated with GAS infection.

### BACKGROUND ART

GAS is a common human pathogen responsible for a large variety of infections which most frequently occur at the level of the upper respiratory tract and skin, causing mild diseases such as pharyngitis, impetigo and cellulitis. Less frequently, GAS infections result in life-threatening and invasive conditions, such as bacteremia, pneumonia, necrotizing fasciitis (NF) and streptococcal toxic shock syndrome (STSS) [1]. In addition, Group A streptococci also lead to non-suppurative sequelae of infections, such as acute rheumatic fever and post-streptococcal glomerulonephritis, which appear to be associated with autoimmunity reactions due to “molecular mimicry” between host tissues and M protein, the major GAS surface-associated antigen. In fact, anti-M specific antibodies have been shown to cross-react with human tissues, including the heart, skeletal muscle, brain and glomerular basement membranes [2].

Post-infectious disorders secondary to GAS infections, especially rheumatic fever, may present with a wide array of neurological and psychiatric pictures, characterized by the association of movement disorders (mainly chorea and tics) and behavioural disorders (mainly obsessive-compulsive symptoms, anxiety and mood disorders) [3]. The nosography of post-streptococcal neuropsychiatric disorders is still in progress. Two entities are however universally acknowledged: Sydenham's chorea (SC) [4], which constitutes one of the major criteria for the diagnosis of acute rheumatic fever, and post-streptococcal acute disseminated encephalomyelitis (PSADEM) [5].

In the last 15 years, it has been suggested that the clinical spectrum of these disorders might be broader. Particularly, in 1998 Swedo et al. proposed the existence of a paediatric disorder mainly characterized by tics and obsessive-compulsive symptoms exacerbating in association with relapses of streptococcal pharyngitis [6]. They indicated this phenotype with the acronym PANDAS (Paediatric Autoimmune Neuropsychiatric Disorders Associated with Streptococcal infections). This hypothesis, however, has generated significant controversy, one critical issue being represented by the difficulty in confirming a temporal relationship between neuropsychiatric symptoms and GAS infections ([7], [8], [9]).

GAS infection is currently diagnosed by culture of throat swabs on blood agar and by determination of the anti-streptolysin O antibody titre (ASO titre) in serum. Streptolysin O is a toxin released by GAS that induces an immune response and detection of antibodies to this protein can thus be used to confirm a recent infection. To date, however, no assays are available for identifying individuals having or at risk of developing disorders secondary to GAS infection. In particular, in the absence of a proven link between GAS infection and neuropsychiatric or behavioural disorders, there are no assays that can be used to identify individuals having or at risk of developing neuropsychiatric or behavioural disorders as a result of GAS infection.

### DISCLOSURE OF THE INVENTION

The invention concerns methods of identifying individuals having or at risk of developing neuropsychiatric or behavioural disorders, in particular tic disorders, resulting from GAS infection. The invention also concerns protein arrays that can be used in such methods.

The invention provides a method of identifying a neuropsychiatric or behavioural disorder in a patient as being associated with GAS infection, or of identifying a patient at risk of developing a neuropsychiatric or behavioural disorder associated with GAS infection, said method comprising the steps of:
a) contacting a biological sample from a patient suffering from or at risk of a neuropsychiatric or behavioural disorder with at least one GAS antigen selected from the group comprising the amino acid sequences of SEQ ID NO:41, SEQ ID NO:45, SEQ ID NO:23, SEQ ID NO:19 or SEQ ID NO:44, or functional equivalents thereof, under conditions appropriate for binding of any antibodies present in the biological sample to the at least one GAS antigen or to the functional equivalents thereof; and
b) detecting the presence of any antibodies in the biological sample bound to the at least one GAS antigen or to the functional equivalents thereof,
wherein the detection of antibodies bound to the at least one of the GAS antigen is indicative that the patient is suffering from a neuropsychiatric or behavioural disorder associated with GAS infection or that the patient is at risk of developing a neuropsychiatric or behavioural disorder associated with GAS infection.

The term “neuropsychiatric or behavioural disorder” as used herein includes movement disorders, such as chorea and tics, and behavioural disorders such as obsessive-compulsive disorder, anxiety and mood disorders. In particular, the methods of the invention may be useful in identifying patients suffering from or at risk of developing GAS-associated tic disorders.

The term “tic disorder” includes motor or phonic tics. The term “tic disorder” also includes: transient tic disorder consisting of multiple motor and/or phonic tics with duration of at least 4 weeks, but less than 12 months; chronic tic disorder consisting of either single or multiple motor or phonic tics, present for more than a year; Tourette's disorder diagnosed when both motor and phonic tics are present for more than a year, and Tic Disorder NOS (not otherwise specified) diagnosed when tics are present, but do not meet the criteria for any specific tic disorder. In some cases, the tic disorder may be associated with other disorders, such as the behavioural and neuropsychiatric disorders discussed above.

Analysis of serum samples from patients affected by tic disorders has led to the surprising finding that the five GAS antigens having the amino acid sequences of SEQ ID NO:41 (SPy0453), SEQ ID NO:45 (SPy1939), SEQ ID NO:23 (SPy1795), SEQ ID NO:19 (SPy1054) or SEQ ID NO:44 (SPy1306) are recognised by significantly higher percentages of sera from patients suffering from tic disorders compared to sera from patients with no tic disorders and sera from patients with pharyngitis. (The SPy numbers given herein are based on the SF370 GAS genome annotation in reference [10].) These findings provide the first evidence that sera from tic patients exhibit immunological profiles typical of individuals who elicited a specific and strong immune response to GAS. These GAS antigens can thus be used to detect the presence of antibodies in patient sera to identify tic disorders and other neuropsychiatric or behavioural disorders associated with GAS infection, and to identify patients with an increased risk of developing tic disorders and other neuropsychiatric or behavioural disorders associated with GAS infection.

The methods of the invention may comprise contacting the sample with 1, 2, 3, 4 or all 5 of the GAS antigens or functional equivalents thereof recited above. Hence, when the sample is contacted with 1 of the GAS antigens, the methods may comprise contacting the sample with: SEQ ID NO:41; SEQ ID NO:45; SEQ ID NO:23; SEQ ID NO:19; or SEQ ID NO:44, or functional equivalents thereof.

Where the sample is contacted with 2 of the GAS antigens, the methods may comprise contacting the sample with: i) SEQ ID NO:41 and SEQ ID NO:45; ii) SEQ ID NO:41 and SEQ ID NO:23; iii) SEQ ID NO:41 and SEQ ID NO:19; iv) SEQ ID NO:41 and SEQ ID NO:44; v) SEQ ID NO:45 and SEQ ID NO:23; vi) SEQ ID NO:45 and SEQ ID NO:19; vii) SEQ ID NO:45 and SEQ ID NO:44; viii) SEQ ID NO:23 and SEQ ID NO:19; ix) SEQ ID NO:23 and SEQ ID NO:44, or x) SEQ ID NO:19 and SEQ ID NO:44, or functional equivalents thereof.

Where the sample is contacted with 3 of the GAS antigens, the methods may comprise contacting the sample with: i) SEQ ID NO:41, SEQ ID NO:45 and SEQ ID NO:23; ii) SEQ ID NO:41, SEQ ID NO:23 and SEQ ID NO:19; iii) SEQ ID NO:41, SEQ ID NO:19 and SEQ ID NO:44; iv) SEQ ID NO:45, SEQ ID NO:23 and SEQ ID NO:19; v) SEQ ID NO:45, SEQ ID NO:19 and SEQ ID NO:44, or vi) SEQ ID NO:23, SEQ ID NO:19 and SEQ ID NO:44, or functional equivalents thereof.

Where the sample is contacted with 4 of the GAS antigens, the methods may comprise contacting the sample with: i) SEQ ID NO:41, SEQ ID NO:45, SEQ ID NO:23, and SEQ ID NO:19; ii) SEQ ID NO:41, SEQ ID NO:23, SEQ ID NO:19 and SEQ ID NO:44; or iii) SEQ ID NO:45, SEQ ID NO:23, SEQ ID NO:19 and SEQ ID NO:44, or functional equivalents thereof.

Alternatively, the sample may be contacted with all 5 of the GAS antigens, i.e. with SEQ ID NO:41, SEQ ID NO:45, SEQ ID NO:23, SEQ ID NO:19 and SEQ ID NO:44, or functional equivalents thereof.

The detection of antibodies bound to 1, 2, 3, 4, or all 5 of these GAS antigens or functional equivalents thereof in a biological sample from a patient with a neuropsychiatric or behavioural disorder, such as a tic disorder, is indicative that the neuropsychiatric or behavioural disorder is associated with GAS infection. The detection of antibodies bound to 1, 2, 3, 4, or all 5 of these GAS antigens or functional equivalents thereof in a biological sample from a patient not having a neuropsychiatric or behavioural disorder, is indicative that the patient is at increased risk of developing a neuropsychiatric or behavioural disorder associated with GAS infection, such as a tic disorder. Suitable methods for detecting the presence of antibodies are known in the art and are described in more detail below.

The methods of the invention may further comprise contacting the sample with further GAS antigens, or functional equivalents thereof, in addition to one or more of the GAS antigens of SEQ ID NOS:41; 45; 23; 19; or 44, and detecting the presence of antibodies binding to these further GAS antigens.

Such further GAS antigens may be selected from the group comprising the amino acid sequences recited in SEQ ID NO:31 (SPy0747), SEQ ID NO:2 (SPy0031), SEQ ID NO:17 (SPy1032), SEQ ID NO:3 (SPy0159), SEQ ID NO:16 (SPy0737), SEQ ID NO:47 (SPy0793), SEQ ID NO:18 (SPy1037), SEQ ID NO:5 (SPy0714), SEQ ID NO:27 (SPy2000), SEQ ID NO:21 (SPy1390), SEQ ID NO:29 (SPy2037), SEQ ID NO:5 (SPy0252), SEQ ID NO:25 (SPy1882), SEQ ID NO:35 (SPy1983), SEQ ID NO:9 (M5005_SPy0249), SEQ ID NO:8 (SPy0287), SEQ ID NO:13 (SPy0441), SEQ ID NO:28 (SPy2007), SEQ ID NO:33 (SPy1326), SEQ ID NO:30 (SPy2066), SEQ ID NO:48 (SPy0838), or functional equivalents thereof.

These 21 GAS antigens have been identified as preferentially reacting against sera from patients suffering from tic disorders and from patients suffering from pharyngitis compared to sera from patients not suffering from tic disorders. Detection of antibodies binding to one or more of these additional GAS antigens, or functional equivalents thereof, in a biological sample, in addition to the detection of antibodies binding to one or more to the GAS antigens of SEQ ID NOS:41; 45; 23; 19; or 44 above may thus be a further indication that a neuropsychiatric or behavioural disorder, such as a tic disorder, is associated with GAS infection, or that a patient is at increased risk of developing a neuropsychiatric or behavioural disorder, such as a tic disorder, associated with GAS infection.

The methods of the invention may comprise contacting the biological sample with 1, 2, 3, 4, 5, 6, 7, 8, 9, 10, 11, 12, 13, 14, 15, 16, 17, 18, 19, 20 or all 21 of these additional GAS antigens of SEQ ID NOS: 31, 2, 17, 3, 16, 47, 18, 5, 27, 21, 29, 5, 25, 35, 9, 8, 13, 28, 33, 30, or 48 under conditions appropriate for binding of any antibodies present in the biological sample to the additional GAS antigens or to the functional equivalents thereof, and detecting the presence of antibodies bound to these further GAS antigens. Further GAS antigens may also be selected from the group comprising the amino acid sequences recited in SEQ ID NO:6 (SPy0269), SEQ ID NO:53 (gi-507127), SEQ ID NO:37 (SPy2010), SEQ ID NO:51 (MGAS10270_SPy1784), SEQ ID NO:24 (SPy1801), SEQ ID NO:46 (SPy1813), SEQ ID NO:26 (SPy1979), SEQ ID NO:20 (SPy1361), SEQ ID NO:49 (gi-126660), SEQ ID NO:4 (SPy0167), SEQ ID NO:32 (SPy0843), SEQ ID NO:42 (SPy0857), SEQ ID NO:50 (gi-4586375), SEQ ID NO:39 (SPy2025), SEQ ID NO:52 (SPyM3_{—}1727), SEQ ID NO:10 (SPy0416), SEQ ID NO:40 (SPy2043), SEQ ID NO:38 (SPy2018), SEQ ID NO:1 (SPy0019), SEQ ID NO:34 (SPy1972), SEQ ID NO:14 (SPy0457), SEQ ID NO:36 (SPy2009), SEQ ID NO:22 (SPy1733), SEQ ID NO:12 (SPy0436), SEQ ID NO:43 (SPy1007), or functional equivalents thereof.

These 25 GAS antigens have been identified as reacting equally against tic, pharyngitis and non-tic sera. These 25 GAS antigens include the SLO protein currently used in GAS diagnosis. Detection of antibodies binding to these antigens, or failure to detect antibodies binding to these antigens in the sample, can thus be used for control purposes. For example, detection of antibodies binding to one or more of these additional GAS antigens, or functional equivalents thereof, in addition to the detection of antibodies binding to one or more of the GAS antigens of SEQ ID NOS:41; 45; 23; 19; or 44 recited above (and optionally the detection of antibodies binding to one or more of the further 21 GAS antigens of SEQ ID NOS: 31, 2, 17, 3, 16, 47, 18, 5, 27, 21, 29, 5, 25, 35, 9, 8, 13, 28, 33, 30, or 48 recited above) serves as a positive control that the patient is or has been infected by GAS. Detection of antibodies binding to one or more of these additional 25 GAS antigens of SEQ ID NOS:6, 53, 37, 51, 24, 46, 26, 20, 49, 4, 32, 42, 50, 39, 52, 10, 40, 38, 1, 34, 14, 36, 22, 12, or 43, or functional equivalents thereof, in the absence of the detection of antibodies binding to one or more of the GAS antigens of SEQ ID NOS:41; 45; 23; 19; or 44 recited above, provides an indication of GAS infection without an associated probability of developing a neuropsychiatric or behavioural disorder associated with GAS. The failure to detect antibodies against one or more of these additional GAS antigens of SEQ ID NOS:6, 53, 37, 51, 24, 46, 26, 20, 49, 4, 32, 42, 50, 39, 52, 10, 40, 38, 1, 34, 14, 36, 22, 12, or 43 provides confirmation that the patient is not suffering from a GAS infection.

The methods of the invention may thus comprise contacting the biological sample with 1, 2, 3, 4, 5, 6, 7, 8, 9, 10, 11, 12, 13, 14, 15, 16, 17, 18, 19, 20, 21, 22, 23, 24 or all 25 of these additional GAS antigens of SEQ ID NOS:6, 53, 37, 51, 24, 46, 26, 20, 49, 4, 32, 42, 50, 39, 52, 10, 40, 38, 1, 34, 14, 36, 22, 12, or 43, or functional equivalents thereof, under conditions appropriate for binding of any antibodies present in the biological sample to the additional GAS antigens or to the functional equivalents thereof, and detecting the presence of antibodies bound to these additional GAS antigens.

Antibody Detection:

The methods of the invention described above all comprise the detection of antibodies bound to the GAS antigens. Methods for detecting antibodies bound to antigens are well known to those of skill in the art and any such methods may be used.

For example, the GAS antigen or antigens (or functional equivalent) may be immobilised at known locations on a surface, such as on the surface of an array as described below. The immobilised antigens may be incubated with the immobilised antigens under conditions that allow the binding of any antibodies present in the sample to the antigens. A suitable incubation period may be around 1 hour. Following washing to remove any unbound antibodies, the detection of antibodies bound to the antigens may be accomplished using an entity that will bind and recognise the bound antibodies.

For example, the step of detecting antibodies bound to the GAS antigens in any of the methods described above may comprise contacting the biological sample and GAS antigens with a labelled secondary antibody, such as a labelled anti-IgG antibody, under conditions suitable for the binding of the secondary antibody to any antibodies in the biological sample that have bound to the immobilised GAS antigens.

The secondary antibody, such as the anti-IgG antibody, may be labelled with a fluorescent or an enzyme label such that the binding of the secondary antibody, and thus the presence of antibodies against the GAS antigens in the biological sample, is detected by detecting the label. Where the label is a fluorescent label, a fluorescence intensity of at least 15,000 may be indicative of a positive result, i.e. of the presence of an antibody in the sample bound to the GAS antigen.

Biological Samples:

The biological samples that may be tested in the methods of the invention may be any sample known to contain antibodies against GAS antigens. Examples of suitable samples are saliva samples, blood samples or serum samples. In particular, the sample may be a serum sample.

The biological sample is from a human patient. The human patient may be an adult, an adolescent between the ages of around 12 to around 18 or from a child under 12. The patient may be displaying clinical symptoms of neuropsychiatric or behavioural disorders, particularly of tic disorders. Alternatively, or in addition, the patient may be displaying clinical symptoms associated with GAS infection, such as pharyngitis. In some cases, the patient may be asymptomatic for current GAS infection.

The methods of the invention may be conducted in vitro. The methods of the first and second aspects of the invention may further comprise the step of obtaining the biological sample from the patient.

Protein Arrays:

In order to facilitate the screening of biological samples against multiple GAS antigens simultaneously, the GAS antigens employed in the methods of the invention may be displayed on one or more protein arrays. For example, each GAS antigens may be displayed on a separate array or a single array may display multiple GAS antigens simultaneously. According to a further aspect of the invention, protein arrays are provided. These arrays are suitable for use in any of the methods described above.

The invention provides a protein array comprising at least two GAS antigens having an amino acid sequence selected from SEQ ID NO:41, SEQ ID NO:45, SEQ ID NO:23, SEQ ID NO:19 or SEQ ID NO:44, or a functional equivalent thereof.

The protein array may comprise 2, 3, 4 or all 5 of these GAS antigens or functional equivalents thereof.

When the array comprises 2 of the GAS antigens, it may comprise GAS antigens having the sequence of: i) SEQ ID NO:41 and SEQ ID NO:45; ii) SEQ ID NO:41 and SEQ ID NO:23; iii) SEQ ID NO:41 and SEQ ID NO:19; iv) SEQ ID NO:41 and SEQ ID NO:44; v) SEQ ID NO:45 and SEQ ID NO:23; vi) SEQ ID NO:45 and SEQ ID NO:19; vii) SEQ ID NO:45 and SEQ ID NO:44; viii) SEQ ID NO:23 and SEQ ID NO:19; ix) SEQ ID NO:23 and SEQ ID NO:44, or x) SEQ ID NO:19 and SEQ ID NO:44, or functional equivalents thereof.

When the array comprises 3 of the GAS antigens, it may comprise GAS antigens having the sequence of: i) SEQ ID NO:41, SEQ ID NO:45 and SEQ ID NO:23; ii) SEQ ID NO:41, SEQ ID NO:23 and SEQ ID NO:19; iii) SEQ ID NO:41, SEQ ID NO:19 and SEQ ID NO:44; iv) SEQ ID NO:45, SEQ ID NO:23 and SEQ ID NO:19; v) SEQ ID NO:45, SEQ ID NO:19 and SEQ ID NO:44, or vi) SEQ ID NO:23, SEQ ID NO:19 and SEQ ID NO:44, or functional equivalents thereof.

When the array comprises 4 of the GAS antigens, it may comprise GAS antigens having the sequence of: i) SEQ ID NO:41, SEQ ID NO:45, SEQ ID NO:23, and SEQ ID NO:19; ii) SEQ ID NO:41, SEQ ID NO:23, SEQ ID NO:19 and SEQ ID NO:44; or iii) SEQ ID NO:45, SEQ ID NO:23, SEQ ID NO:19 and SEQ ID NO:44, or functional equivalents thereof.

Alternatively, the array may comprise all 5 of the GAS antigens, i.e. SEQ ID NO:41, SEQ ID NO:45, SEQ ID NO:23, SEQ ID NO:19 and SEQ ID NO:44, or functional equivalents thereof.

The protein array may comprise additional GAS antigens that may be selected from the group comprising the amino acid sequences recited in SEQ ID NOS:31, 2, 17, 3, 16, 47, 18, 5, 27, 21, 29, 5, 25, 35, 9, 8, 13, 28, 33, 30, 48 or functional equivalents thereof. The array may comprise 1, 2, 3, 4, 5, 6, 7, 8, 9, 10, 11, 12, 13, 14, 15, 16, 17, 18, 19, 20 or all 21 of these additional GAS antigens.

The protein array may comprise additional GAS antigens that may be selected from the group comprising the amino acid sequences recited in SEQ ID NOS 6, 53, 37, 51, 24, 46, 26, 20, 49, 4, 32, 42, 50, 39, 52, 10, 40, 38, 1, 34, 14, 36, 22, 12, 43, or functional equivalents thereof. The array may comprise 1, 2, 3, 4, 5, 6, 7, 8, 9, 10, 11, 12, 13, 14, 15, 16, 17, 18, 19, 20, 21, 22, 23, 24 or all 25 of these additional GAS antigens.

Any type of protein array known in the art may be used in the method of invention. Production of protein arrays is described in references 11, 12.

For example, the protein array may be a glass slide to which the antigen or antigens are anchored. In its simplest form, the array may be a glass slide displaying a simple antigen prepared simply by coating glass microscope slides with aminosilane (Ansorge, Faulstich), adding an antigen-containing solution to the slide and drying. Slides coated with aminosilane may be obtained from Telechem and Pierce for coating with the antigen.

Alternatively, the array may display multiple antigens. For example, nitrocellulose-coated slides may be spotted with nanoliters of multiple GAS antigens. Such arrays may display replicates of each GAS antigen. The antigens spots in such arrays may be approximately 150 μm in diameter and contain ˜0.35 ng of protein

Other types of protein array include a 3D gel pad and microwell arrays. As will be apparent to the skilled reader, types of protein array that have not yet been conceived but which are devised in the future may well prove to be suitable for use in accordance with the present invention.

The invention further provides a kit comprising a protein array according to the invention and instructions for the use of the array in the identification of patients having or at risk of developing a neuropsychiatric or behavioural disorder associated with GAS infection.

Functional Equivalents:

The SEQ ID NOS used to identify the GAS antigens that may be used in the methods and protein arrays of the invention described above are full length sequences for these GAS antigens.

The methods and protein arrays of the invention are not limited to the use of these full-length GAS antigens but also encompass any “functional equivalent” of any of these GAS antigens.

The term “functional equivalent” as used herein is intended to encompass variants of the GAS antigens having the full-length sequences shown in the sequence listing that retain the ability to interact with antibodies against the full-length GAS antigen present in the biological and that may thus be used in place of the full-length GAS antigens.

The term “functional equivalent” thus encompasses fragments of the full-length GAS antigens having the sequences shown in the sequence listing. Such fragments may retain the ability to bind to antibodies that bind to the full-length GAS antigens. The functional equivalents of the invention may bind to antibodies generated against the full-length GAS antigen with an affinity of at least 10⁻⁷M.

Fragments include at least n consecutive amino acids of the full-length GAS antigen sequences, wherein n is 7 or more (e.g. 8, 10, 12, 14, 16, 18, 20, 25, 30, 35, 40, 50, 60, 70, 80, 90, 100, 150, 200, 250 or more). Fragments may comprise an epitope from the full-length GAS antigen sequence. Further fragments may lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of the full-length sequence. For example, fragments that may be employed in the methods and arrays of the invention include fragments that are lacking the leader sequences and/or the transmembrane sequences present in the full-length GAS antigens. The portions of the GAS antigens that are considered to be leader sequences and transmembrane sequences are shown in the sequence listing.

Further examples of fragments that may be used in the methods and arrays of the invention include N-terminal fragments. Examples of such fragments include the amino acid sequence shown in SEQ ID NO:11 (which is an N-terminal fragment of the sequence in SEQ ID NO:10) and the amino acid sequence shown in SEQ ID NO:7 (which is an N-terminal fragment of SEQ ID NO;6).

The term “functional equivalent” also includes variants of the full-length GAS proteins having amino acid substitutions and fragments of such variants. Variants may have 50% or more identity (e.g. 60%, 65%, 70%, 75%, 80%, 85%, 90%, 91%, 92%, 93%, 94%, 95%, 96%, 97%, 98%, 99%, 99.5% or more) to the full-length GAS antigen sequences provided herein. Variants may contain conservative amino acid substitutions compared to the GAS antigen sequence given in the sequence listing. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr.

The term “functional equivalent” additionally encompasses longer variants of the GAS antigens including fusion proteins that include an additionally entity that has been chemically or genetically linked to the GAS antigen. For example, the GAS antigen may be attached a label that facilitates its localisation on a protein array or facilitates detecting it when it is bound to an antibody. Examples of such labels include an analytically-detectable reagent such as a radioisotope, a fluorescent molecule or an enzyme. Alternatively, the GAS antigen may be fused to a domain that facilitated its initial purification, such as a histidine or GST domain.

The term “functional equivalent” also includes mimetics of the GAS antigens, variants and fragments described above, which are structurally similar to the GAS antigens and retain the ability to bind to antibodies against the full-length GAS antigens.

General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Identity between polypeptide sequences is preferably determined by the Smith-Waterman homology search algorithm as implemented in the MPSRCH program (Oxford Molecular), using an affme gap search with parameters gap open penalty=12 and gap extension penalty=1.

### BRIEF DESCRIPTION OF THE DRAWINGS

FIG. 1. Protein micro array set-up and validation. A, SDS-PAGE analysis of purified recombinant GAS proteins stained with Coomassie. Molecular weight markers in lane 1. B, Representative image of a chip after incubation with a human serum and with Cy3-labelled anti-human IgG and Cy5-labelled anti-human IgM. Replicates of tested antigens and of negative and positive IgG and IgM controls are highlighted. C, graphic representation of the control human IgG curve. Orange dots correspond to the different IgG concentrations measured on the x-axis, while the continuous line corresponds to the interpolated resulting curve. MFIs values are reported on the y-axis.

The chip image of different IgG concentration revealed by incubation with anti-human IgG-Cy3 is shown below the graph. D, Sigmoid-derived data normalization method. Data are normalized using the sigmoid control curve (black) referred to a reference sigmoid curve (red). IgG control concentrations and MFIs are reported on the x- and the y-axis respectively. HL, high level signal area (normalized MFI value >40,000); LL, low level signal area (normalized MFI value <15,000); id, ideal sigmoid curve; P and P′, intersection points of not normalized and normalized MFI values on the experimental and reference sigmoid curve; Val and N (Val): Background-subtracted MFI value and normalized value resulting after normalization, respectively.

FIG. 2. Tic, No Tic and Pharyngitis sera analysis. A, Unsupervised hierarchical clustering of human Tic (n=61), No Tic (n=35) and Pharyngitis (n=239) sera versus the selected GAS antigens. Antigens/sera interactions resulting in signals with high or low FI are visualized in yellow and blue respectively. Color scale of signal intensity is reported on top-left. Clusters group antigens and/or sera showing similar reactivity profiles. Colored bars and roman numbers on the right of the dendrogram identify the sera clusters considered for the analysis. B, Distribution of sera in the different clusters. The histogram shows the percent of positive sera (FI>15000) for each class in relation to the five defined clusters.

FIG. 3. Correlation between sera ELISA titers and MFI. Bars indicate the geometric mean values (left y-axis) of ELISA titers obtained using 8 sera from either no tic (black bars) or tic (white bars) patients for the four antigens indicated below the x axis. Asterisks above white bars indicate statistically significant differences between tic and no tic ELISA titers. White circles (no tic sera) and black triangles (tic sera) indicate the arithmetic mean of the MFIs (right y-axis) obtained on the protein chip with the same sera. Both ELISA titers and MFIs differences were statistically significant with a P values<0.05 calculated either with T Student (ELISA) or Fisher's exact test (MFI).

FIG. 4. Relative reactivity of the three sera classes. A, number of tested GAS antigens recognized by at least 30% of sera for each patient group. B, Percentages of antigens recognized by at least 30% of the sera of each class with FI>40,000. C, percentages of sera for each class reacting with at least 30% of the antigens and with FI>40,000. Numbers above histograms bars indicate P values calculated with the two-tailed χ² test.

### MODES FOR CARRYING OUT THE INVENTION

Introduction

In the attempt to shed some light on the possible contribution of GAS infections to the onset of neuropsychiatric or behavioral disorders, we concentrated our attention on a group of young patients with tic disorder, a pathological condition consisting of “sudden, repetitive, non-rhythmic, involuntary movements (motor tic) or sounds (phonic tic) that involve discrete groups of muscles” [13]. Sera obtained from these children were tested for their immunological reactivity versus a representative panel of GAS antigens. The analysis was carried out using the protein array technology [12], which allows high throughput analysis of human sera against a large number of antigens. Protein chips containing 102 GAS proteins were probed with sera from tic patients and children without tics. The resulting data were compared with those obtained with chips probed with GAS-associated pharyngitis patient sera. By using this experimental approach, we were able to better define the relationship between tic disorder and immune response to GAS antigens.

Results

Micro Array Design and Validation

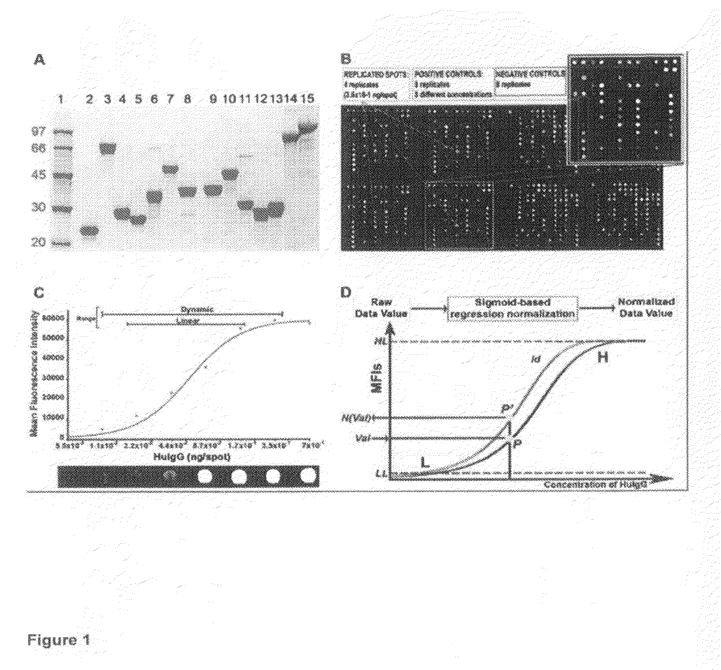

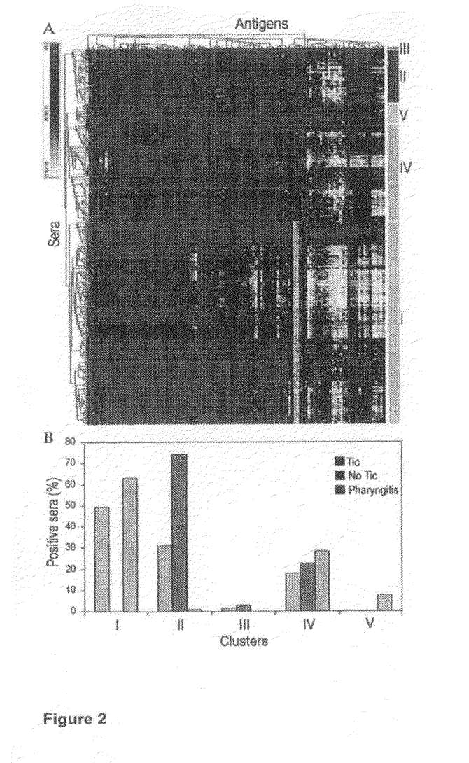

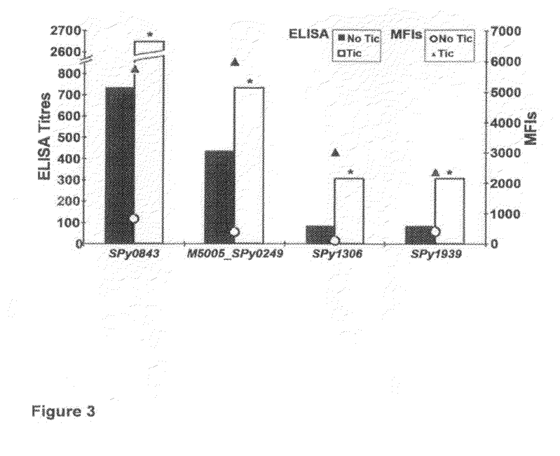

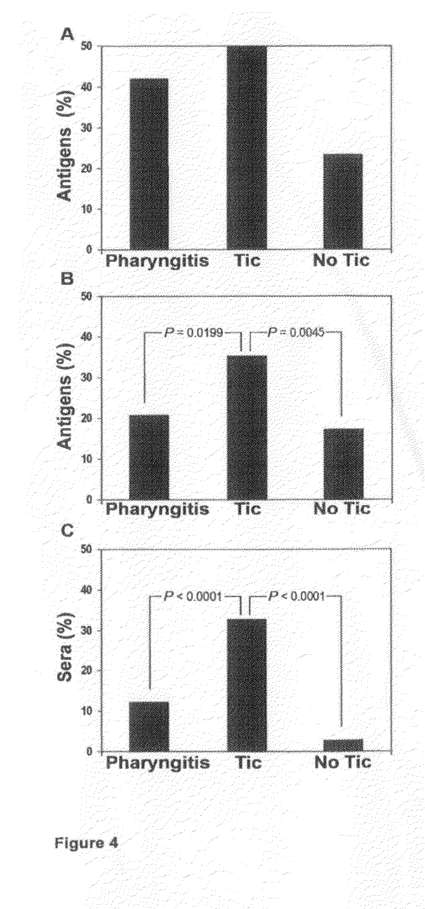

To study the serological response of tic patient versus a representative panel of GAS antigens a protein array was generated by printing 102 recombinant proteins, mainly selected from the GAS SF370 M1 genome (Table 1). The majority of printed GAS proteins were expressed as C-terminal His-tag fusions while 23 proteins where expressed as double fusions, with glutathione S-transferase (GST) at the N-terminal and with a His-tag at the C-terminal. Proteins obtained after affinity purification from the bacterial soluble fraction showed purity levels equal or greater than 70%, as estimated by densitometric scan of PAGE-SDS gels (FIG. 1A). The protein array validation was obtained by using a defined printing scheme and control spots (FIG. 1B). Printing 4 replicates of each antigen followed by incubation with mouse anti-sera raised against the recombinant proteins and/or GST and His fusion tags, assured that all of them were efficiently and reproducibly immobilized on nitrocellulose slides (not shown). PBS buffer, spotted on either side of each protein spot was used to detect protein carry-over during spotting and fewer than 10% of PBS spots showed signal intensities higher than the average nitrocellulose background fluorescence intensity (FI) value (see Materials and Methods). Proteins eluted after affinity purification of total soluble extracts from an E. coli strain carrying the empty expression vectors were also printed on the array to determine the maximum background signal due to contaminants possibly generated by incubation with the sera under investigation, which always resulted in a mean fluorescence intensity (MFI) not higher than 1023 (Standard deviation, SD, 461). Finally, 8 different amounts of human IgGs were spotted on the array in 4 replicates (from 6×10⁻³ to 7×10⁻¹ ng of immobilized protein per spot) and used as controls for detection, system reproducibility and data normalization. MFIs of human IgG spots, obtained after detection with Cy3-conjugated anti-human antibodies, were best fitted by sigmoid curves showing a signal dynamic range of about 2 logs and, within this range, a linearity covering approximately 1 log of FI values, with a lower detection limit which resulted to be approximately 7×10⁻³ ng (FIG. 1C). To compare data from different experiments we used a normalization method previously set up and validated for our system (Reguzzi V., personal communication), in which the experimental IgG curve of each slide was adjusted on a reference sigmoid IgG curve, and the background-subtracted MFI values of each protein were normalized accordingly (FIG. 1D and Materials and Methods). The definition of the MFI of the reference IgG curve also permitted us to assess that the intra-slide coefficients of variation (CV) was lower than 5%, while the inter-slide CV, measured by computation of human IgG MFI values of 50 slides, ranged from 46% to 30% for FI values lower than 15000 (value corresponding to the normalized MFI value of buffer spots plus 2 standard deviations as described in Materials and Methods) and decreased to approximately 20% for FI values higher than 15000. On the basis of these results, a normalized FI value of 15000 was arbitrarily chosen as the lowest signal threshold for scoring a protein as positively recognized by human sera. In addition, a second FI cut off of 40,000 was arbitrarily defined to mark highly reactive proteins and/or identify high titer sera, which in our experimental system corresponded approximately to the saturation segment of the human IgG curve (FIG. 1D).

Serological Profiling of Tic, no Tic and Pharyngitis Sera.

In the attempt to determine if there is a link between GAS infection or exposure to GAS antigens and tic disorders, the GAS protein array was probed with 61 sera from patients with tics. At the time of the visit patients did not show clinical signs of pharyngitis and the percentage of GAS carriers in the group (14.7%) did not differ from that observed in a normal children population falling in the same age range [14]. A similar analysis was carried out using 35 sera from children without tic disorder and without pharyngitis symptoms (referred to as “no tic”) and 239 sera from GAS-pharyngitis patients (confirmed by throat swab and isolation of the infective GAS strain). All groups were formed by 4-14-year-old children. Sera reactivity was evaluated by detecting total IgG bound to each protein spot using fluorescently labeled anti-human IgG and measuring the FI values for each antigen. To define the antigen recognition pattern of the three sera groups, the normalized FI values were subjected to unsupervised bi-dimensional hierarchical clustering using the Pearson algorithm to calculate cluster distances. The clustered view of the antibody recognition profiles of the GAS antigens observed for tic, no tic and pharyngitis patients, for a total of 335 sera, is shown in FIG. 2A. This analysis resulted in the definition of 5 different major clusters at the second level of hierarchy. Cluster I included 181 sera, cluster II 47 sera, cluster IV 87 sera. Clusters III and V included a minority of the sera (2 and 18 respectively). Most of the tic sera distributed without any statistically significant difference in clusters I, II and IV, while pharyngitis sera were grouped in clusters I, IV and V (FIG. 2B). Remarkably, nearly 80% of the no tic sera segregated in cluster II, indicating that the large majority of sera belonging to the negative control group had a significantly different antigen recognition pattern as compared to the other two sera classes.

Specific Immunogenicity of GAS Antigens in Tic, no Tic and Pharyngitis Patients.

When we analyzed the tic sera reactivity frequencies for each single antigen, 50% of them resulted to be recognized by at least 30% of the tic patient sera, while approximately 25% resulted to be reactive against less than 10% of them. Three groups of immunogenic antigens could be distinguished: (i) one consisting of 25 proteins (Table 1), which were recognized with similar frequencies by all three sera families. Several known immunogenic GAS antigens fell in this subset, such as M proteins, streptolysin O (SLO), streptokinase A and C5a peptidase precursors. The fact that these antigens were also recognized by sera belonging to the no tic negative control group, was not considered contradictory since these individuals had most probably already experienced GAS infections; (ii) another group of 21 proteins (Table 3), recognized with comparable frequencies by both tic and pharyngitis sera but with statistically significant lower frequencies by no tic sera; (iii) a third group of 5 proteins (Table 4), which were instead recognized with statistically significant higher recognition rates by tic sera compared to both pharyngitis and no tic sera. Interestingly, approximately 40% of tic sera were simultaneously reactive against at least 3 of these antigens (Table 5).

The possibility that a correlation existed between the observed serological profiles and an ongoing or recent GAS infection was evaluated by considering the anti streptolysin O (ASO) titers of tic patients. According to our previous works 15 we fixed the cut-off of 407 International Units (I.U.) to define high or low ASO titer. When 31 tic sera with high ASO were compared to 16 tic sera with low ASO, we did not observe major differences neither in the type nor in the total number of recognized GAS antigens (not shown).

To confirm and further validate our data, IgG levels against few test antigens included in the three groups were analyzed by standard methods. When 8 negative sera (MFI<15000) and 8 positive sera (MFI>15000) against each of the antigens SPy0843, M5005_SPy0249, SPy 1306 and SPy 1939 were tested by ELISA, statistically significant titer differences were observed among negative and positive sera, as defined on the basis of the MFIs resulting from the micro-array analysis (FIG. 3).

Comparison of the Immunoreactivity of Tic Sera.

The relationship between tic and anti-GAS immune response was confirmed and strengthened by comparing the overall reactivity of the three sera families against the selected GAS proteins. While the percentages of antigens recognized by >30% of tested sera were not remarkably different for the tic and pharyngitis groups, no tic sera recognized overall fewer antigens (FIG. 4A). Significant divergences between tic sera and the other two sera families were instead observed when the differences in spot intensities were considered, as indicative of differential antigen-specific IgG levels. In fact, we verified which were the percentages of antigens recognized most intensely by the three sera families (FI>40,000, arbitrary cut-off) and, conversely, which the percentages of sera generating on the array a very high signal (FI>40,000) against at least 30% of the tested antigens. The percentage of GAS antigens recognized by the tested sera with FI>40.000 was about 30% in the case of tic patients, 18% in the case of pharyngitis patients and 13% in the no tic group (FIG. 4B). Similarly, over 30% of the tic patient sera reacted very intensively (FI>40,000) with at least 30% of the spotted antigens, as opposite to 12% of the pharyngitis patient sera and only 3% of the negative control group sera (FIG. 4C). In both cases, statistical analysis carried out using the χ² test and referring to a P value<0.05 confirmed that the differences observed between the reactivity of tic sera and the other two sera groups were statistically significant.

Discussion

In this work we have addressed the issue of whether a consistent and strong correlation between tic disorders and GAS infection could be established. To this purpose, we have carried out the first systematic analysis of the IgG response of tic patients sera versus a representative panel of GAS antigens and the resulting data were compared to those obtained with sera from children either without tic or with pharyngitis.

The protein micro array approach which we exploited turned up to be appropriate to test a total of almost 350 human sera against over 100 bacterial antigens, demonstrating that this technology should be taken into account whenever large sets of data on in vivo expression of pathogen antigens and on the subsequent host immune response are required. Protein micro array confirmed to be a fast, easy and sensitive approach and the results that we obtained with the different controls were sufficiently robust to validate data obtained with the biological samples. As a further confirmation, the different MFIs obtained with either positive or negative sera and which were indicative of differential antigen-specific IgG levels, appeared to be fully consistent with the corresponding IgG titers determined by using a more conventional ELISA assay (FIG. 3), confirming what previously observed by Robinson et al.[16], who not only demonstrated that chip and ELISA results correlate but who also highlighted the higher sensitivity of the array approach.

Two major conclusions can be derived from our protein array results. The first one is that the serological profiles observed in tic patients were similar to those observed in sera of patients who experienced a common acute pharyngitis. In fact our analysis established that 46 antigens, out of the 102 present on the chip, reacted against tic and pharyngitis patient sera in a similar manner, being each of them recognized by comparable percentages of sera from the two groups (Table 2 and Table 3). Remarkably, the profiles of both sera groups were significantly different from those observed in no tic patients (Table 3 and FIG. 2).

The second conclusion coming from the array results is that the IgG response of tic sera appeared to be overall quantitatively stronger than that observed in pharyngitis patients. In fact, when we took into account the frequencies of highly reacting antigens and sera (MFIs higher than 40,000), they appeared to be significantly higher in tic patients, compared to both pharyngitis and no tic patients (FIGS. 4B and 4C).

Overall, the results discussed so far demonstrated that a large number of GAS antigens eliciting an immune response in the course of a common acute pharyngitis were also recognized, and even more robustly, by tic sera. These data provide the first evidence that tic patients exhibit serological profiles typical of individuals who have mounted a specific and strong immune response against Group A Streptococcus antigens, strengthening the relationship between tic disorder and GAS infection, so far based only on discordant epidemiological reports and few signs of infection [17]

Given the association between tic disease and GAS infections, it is challenging to envisage to what extent and how the pathogen may contribute to the onset or recurrence of tic disorder. These issues sound even more intriguing if we consider that the immune response against GAS antigens which we analyzed occurred in tic patients in the presence of neuropsychiatric symptoms but in the absence of overt GAS infection, as testified by the lack of clinical signs of pharyngitis and by the usual GAS carrier frequency observed in the tic patient population. Additionally, the fact that no major differences were observed between tic patients with low or high ASO titers further suggested that the serological profiles of these patients was not strictly correlated with the immune response against SLO, which is the parameter routinely used to confirm an ongoing or recent GAS-induced pharyngitis.

Still much has to be explained on how GAS infections contribute to the wide spectrum of post-streptococcal syndrome of the central nervous system (CNS) [18]. It is assumed that CNS disease predisposition is dependent on a multiplicity of factors, among which genetic background [19], and that GAS infections “per se” are not the primary cause of the outcome of CNS disorders but contribute as cofactors triggering the disorder outcome or increasing the risk for the disease to occur, especially in the presence of a particular genetic predisposition.

Induction of non-suppurative sequelae due to “molecular mimicry” between bacterial antigens and host tissues distinguishes Group A Streptococcus from related streptococci and most of other human bacterial pathogens. Protein M is the prototype antigen causing “autoimmune sequelae”, primarily represented by acute RF and by rheumatic heart disease (RHD). These pathologies have indeed been linked to mimicry between M protein and cardiac myosin, due to the immunological response to GAS infection which would cause induction of specific cross-reacting antibodies and inflammatory T cells infiltrating and damaging the myocardium or valve [20, 21]. Similar structural and immunological similarities have been found between M protein and several other auto antigens such as tropomyosin, vimentin, keratin and laminin [1]. The glycolytic enolase enzyme has also been identified as an additional antigen, possibly playing a role in acute RF development. In fact, antistreptococcal enolase antibodies appeared to cross-react with human enolase and sera from acute RF patients had higher anti-human and anti-bacterial enolase titers compared to those found in sera of both pharyngitis and healthy control subjects [22]. Similarly, antibody titers against 5 streptococcal antigens and 4 tissue antigens possibly involved in molecular mimicry, were recently shown to be significantly higher in acute RF compared to pharyngitis patients [23].

The possibility that post-streptococcal autoimmune events similar to those mentioned above could play a role in causing or contributing to neuropsychiatric disorders is still under evaluation. This hypothesis appeared to be supported by the fact that higher levels of antibodies directed against brains structures were observed in children with OCD and PANDAS [24, 25] and that patients with Tourette's syndrome and tic disorder have increased titers of antibodies specific for streptococcal M protein [26], which is known to elicit antibodies cross-reacting with human brains proteins [27]. Further support to the autoimmune hypothesis derived from the observations that monoclonal antibodies obtained from Sydenham's chorea patients [28] showed specificity for mammalian lysoganglioside and N-acetyl-β-D-glucosamine (GlcNAc), the dominant epitope of group A streptococcal carbohydrate, as well as from data indicating the M1 isoform of pyruvate kinase [29] and additional neuronal surface glycolytic enzymes [30] as autoimmune targets in Tourette syndrome and other CNS diseases.

On the basis of this background, our data demonstrating a strong immune response against GAS antigens in tic patients even in the absence of evident infection, suggest that the serological profile observed in these patients may be relevant in the context of one of the current hypothesis [25,31] proposing that antibodies directed against specific streptococcal antigens could be responsible for auto-immune reactions triggering the occurrence of tic disorders in susceptible individuals. In this scenario, peaks of autoimmune response against critical GAS antigens may occur cyclically, due to repeated boosts, as a consequence of either recurrent infections or re-exposure to the pathogen capable of surviving within the host, causing periodic release and exacerbation of neuropsychiatric or behavioral symptoms in children.

In conclusion, we believe that our data demonstrated the link existing between tic disorder and GAS infection and strengthened the concept that a relationship may exist between tics and antibody response against Group A Streptococcus antigens.

Materials and Methods

Selection, Expression and Purification of GAS Surface-Exposed Antigens

Computer programs included in the GCG Wisconsin Package version 11.1, in combination with PSORT program, were used to analyze the SF370 strain sequence and to select a subset of predicted surface-exposed and secreted proteins. Proteins included in the subset were those containing leader peptides, lipoprotein signature, outer membrane anchoring motives, host cell binding domains such as RGD and homologies to known surface proteins or to known virulence factors. As a result of this activity, 96 genes were selected from SF370, cloned and expressed in E. coli both as a C-terminal His-tag fusion protein or as a double fusion protein with an N-terminal GST peptide and a C-terminal His-tag [32]. Following IMAC or Glutathione-sepharose affinity-chromatography, the antigens were successfully purified from the bacterial soluble fraction, dialyzed against PBS buffer and used to assemble the GAS-specific protein array. In addition 6 emm genes were cloned from strains with different M types and the corresponding proteins were purified and spotted on the chip. The proteins were: M1 from SF370, M2 from 2726 strain, M3 from MGAS315, M9 from 2720 strain, M12 from 2728 strains and M23 from DSM2071. All antigens are listed in the Table 1.

Protein Micro Array Technology

The GAS protein array was generated by spotting affinity-purified recombinant protein (0.5 mg/ml) in 4 replicates on nitrocellulose-coated slides (FAST slides, Schleicher and Schuell) with the Chipwriter Pro spotter (Biorad), resulting in spots of approximately 150 μm in diameter. As experimental positive controls to assess the sensitivity and reproducibility of the array set up and for data normalization, a curve of human IgG(s) (solutions from 0.008 to 1 mg/ml) was spotted on the arrays in 8 replicates and detected with Cy3 conjugated α-human IgG secondary antibody. Similarly, a curve of human IgM(s) was also spotted on the arrays, which were undetectable under tested conditions.

PBS buffer was spotted in at least twice the number of the protein spots, and used to assess the possible non-specific signal due to cross contamination. For experiments with human sera, slides were saturated with blocking solution 3% Top Block (Fluka-BioChemiKa)—0.1% Tween 20 in PBS (TPBS), and later incubated with sera (1:1000 dilutions in TPBS) for 1 h at room temperature. After three washes for 5 min in 0.1% TPBS, slides were incubated with Cy3 conjugated anti-human IgG (Southern Biotech) for 1 h at room temperature in the dark. Afterwards arrays were washed twice with TPBS, once with PBS and finally with milliQ sterile water (30 seconds) and were then dried at 37° C. for 10-20 minutes in the dark and scanned. Each serum was tested at least twice. Spotting was validated by confirming the presence of all immobilized proteins using mouse anti-GST and anti-His6 tags monoclonal antibodies, followed by detection with a Cy3 labeled a-mouse IgG secondary antibody. Fluorescence signals were detected by using a ScanArray 5000 Unit (Packard, Billerica, Mass., USA) and the 16-bit images images were generated with ScanArray™ software at 10 μm per pixel resolution and processed using ImaGene 6.0 software (Biodiscovery Inc, CA, USA). Elaboration and analysis of image raw fluorescence Intensity (FI) data was performed using an in house-developed software. For each sample, the mean fluorescence intensity (MFI) of replicated spots was determined, after subtraction of the background value surrounding each spot, and subsequently normalized on the basis of the human IgG curve to allow comparison of data from different experiments. The MFIs values of IgG, spotted at different concentrations, were best fitted by a curve belonging to sigmoid family using a maximum likelihood estimator [33]. The normalization method has been set up by defining a reference IgG curve that covers the entire 16 bits pixel range, adjusting the experimental IgG curve and normalizing protein MFIs values accordingly. With the following formula we get the normalized MFI value from the experimental MFI value (Reguzzi V., personal communication):

$$Normalized\_Value = LLid + \frac{( HLid - LLid )}{1 + {( \frac{( HL - Value )}{Value - LL )} )}^{\frac{σ}{σ id}} \cdot {e}^{\frac{( μ id - μ )}{σ id}}}$$

where HLid, LLid, σid and μid are the parameters for the reference sigmoid and HL, LL, σid and μid are the parameters for experimental IgG curve.

In order to profile the specificity of the antigenic responses of each serum and to define the antigen recognition pattern of the three classes of sera, the normalized MFI values were subjected to unsupervised bi-dimensional hierarchical clustering using TIGR Multiexperiment Viewer (MeV) software (http://www.tigr.org/software/tm4/mev.html).

Human Sera

“Pharyngitis” sera were collected from 249 male and female patients aged 4-14 with clinical symptoms of pharyngitis. A throat swab was performed at diagnosis which confirmed pharyngitis being GAS-associated and allowed the isolation of the GAS infective strain, which was then serotyped according to the sequence of the specific M protein antigen. Among the isolated strains, all of the epidemiologically most important M types were represented.

“Tic” sera were collected from patients affected by tic disorders during their first observation at outpatient division of The Department of Child and Adolescent Neuropsychiatry of the University of Rome La Sapienza between September 2004 and July 2005. Inclusion criteria comprised to be affected by tic disorders; exclusion criteria comprised mental retardation, pervasive developmental disorders, non ambulant subjects and institutionalized children. In total, 61 consecutive patients (6 females) affected by tic disorder aged 4-14 years (mean: 9.6; median: 9.2) were studied. All patients were coming from Rome and its province. 17 patients were recruited during fall, 20 during winter, 18 during spring and 8 during summer. In the large majority of cases the observation occurred during a period of symptom exacerbation in subjects having had tics for months or years; only 3 patients were examined at the onset of tic symptoms. In these patients the severity of tics, as measured by the Yale Global Tic Severity Scale, ranged between 15 and 35 (without impairment score). No patient showed clinical signs of pharingo-tonsillitis; 9 patients (14.75%) had a pharyngeal swab positive for GAS. All isolates were typed according to the sequence of the M protein antigen (emm typing). The types identified covered several M types with no prevalence of any specific type. Regarding anti-streptolysin O (ASO) titers, 14 patients showed normal values (<200 International Units, I.U.), 16 intermediate values (200-400 I.U.), 19 elevated values (400-1000 I.U.) and 12 very elevated values (>1.000 I.U.) No direct correlation by swab positivity for GAS and ASO titer was observed but the mean value for GAS positive patients was in the range that we classified as elevated (about 650 IU). In all 50.81% of the patients had ASO titers elevated or very elevated. The season of recruitment of patients was not correlated with ASO titer values or with swab positivity for GAS.

“No tic” sera were collected from 35 outpatients (4 females) aged 4-14 years (mean: 8.9; median: 9.1) affected by benign epilepsies attending to The Department of Child and Adolescent Neuropsychiatry of the University of Rome La Sapienza. The controls were matched to the tic patients for sex, age and time (season of the year) of observation. No patient had tic symptoms or pharingo-tonsillitis. Regarding ASO titer, 16 patients showed normal values (<200 I.U.), 15 intermediate values (200-400 I.U.) and then 4 elevated values (400-1000 I.U.). In all 11.42% of the patients had elevated, and none had very elevated ASO titers.

Pharyngitis, tic and no tic sera samples were residual obtained during routine medical controls for GAS pharyngitis diagnosis or during neuropsychiatric visits and were available at Baylor College of Medicine, Houston, Tex., (pharyngitis) or at Istituto Superiore di Sanità, Rome and Department of Child and Adolescent Neuropsychiatry, University La Sapienza, Rome (tic and no tic sera). These Institutions had authorization to treat such residual materials for research purposes.

Statistical Analysis

Statistical differences among sera groups, ELISA titers and MFIs were analyzed using the two-tailed χ², the T student or the Fisher's exact tests. Differences were considered statistically significant when P values below 0.05 were obtained.

TABLE 1
In silicO selected GAS antigens

| SPy^{(a)} | Annotation |
|---|---|
| gi-126660 | M protein type 12 |
| gi-4586375 | M protein type 23 |
| gi-507127 | M protein type 9 |
| MGAS10270_SPy1784 | M protein type 2 |
| M5005_SPy_0107^{(b)} | collagen binding protein |
| SPy0019 | putative secreted protein |
| SPy0031 | putative choline binding protein |
| SPy0128 | hypothetical protein |
| SPy0130 | hypothetical protein |
| SPy0159 | hypothetical protein |
| SPy0163 | putative ABC transporter (lipoprotein) |
| SPy0167 | streptolysin O precursor |
| SPy0186 | hypothetical protein |
| SPy0210 | hypothetical protein |
| SPy0212 | exotoxin G precursor |
| SPy0252 | putative sugar transporter sugar |
| | binding lipoprotein |
| SPy0269 | putative surface exclusion protein |
| SPy0287 | hypothetical protein |
| M5005_SPy0249^{(b)} | oligopeptidepermease OppA |
| SPy0317 | hypothetical protein |
| SPy0380 | putative manganese-dependent inorganic |
| | pyrophosphatase |
| SPy0385 | ferrichrome ABC transporter |
| | (ferrichrome-binding protein) |
| SPy0416 | putative cell envelope proteinase |
| SPy0436 | putative exotoxin (superantigen) |
| SPy0441 | hypothetical protein |
| SPy0453 | metal binding protein of ABC transporter |
| | (lipoprotein) |
| SPy0457 | putative cyclophilin-type protein |
| SPy0513 | putative XAA-PRO dipeptidase; |
| | X-PRO dipeptidase |
| SPy0591 | putative protease |
| SPy0604 | hypothetical protein |
| SPy0652 | hypothetical protein |
| SPy0711 | pyrogenic exotoxin C precursor, |
| | phage associated |
| SPy0712 | putative DNase (similar to mitogenic |
| | factor), phage associated |
| SPy0714 | putative adhesion protein |
| SPy0731 | phosphopyruvate hydratase (enolase) |
| SPy0737 | putative extracellular matrix binding protein |
| SPy0740 | streptolysin S associated ORF |
| SPy0747 | hypothetical protein |
| SPy0772 | hypothetical protein |
| SPy0778 | putative ABC transporter |
| | (substrate-binding protein) |
| SPy0793 | hypothetical protein |
| SPy0838 | hypothetical protein |
| SPy0843 | hypothetical protein |
| SPy0857 | putative peptidoglycan hydrolase |
| SPy0925 | putative oxidoreductase |
| SPy1006 | putative lysin - phage associated |
| SPy1007 | streptococcal exotoxin I |
| SPy1013 | putative fibronectin-binding protein-like |
| | protein A |
| SPy1032 | extracellular hyaluronate lyase |
| SPy1037 | hypothetical protein |
| SPy1054 | putative collagen-like protein |
| SPy1105 | putative spermidine/putrescine ABC |
| | transporter |
| SPy1173 | glucose-inhibited division protein A |
| SPy1204 | bifunctional GMP synthase/glutamine |
| | amidotransferase protein |
| SPy1228 | putative lipoprotein |
| SPy1245 | putative phosphate ABC transporter, |
| | periplasmic phosphate-binding protein |
| SPy1274 | putative amino acid ABC transporter, |
| | periplasmic amino acid-binding protein |
| SPy1280 | D-fructose-6-phosphate amidotransferase |
| SPy1290 | hypothetical protein |
| SPy1294 | putative maltose/maltodextrin-binding |
| | protein |
| SPy1306 | maltose/maltodextrin-binding protein |
| SPy1326 | hypothetical protein |
| SPy1357 | protein GRAB (protein G-related alpha |
| | 2M-binding protein) |
| SPy1361 | putative internalin A precursor |
| SPy1390 | putative protease maturation protein |
| SPy1436 | putative deoxyribonuclease |
| SPy1491 | hypothetical protein |
| SPy1497 | putative hemolysin |
| SPy1558 | hypothetical protein |
| SPy1577 | 3-dehydroquinate synthase |
| SPy1618 | putative O-acetylserine lyase |
| SPy1633 | hypothetical protein |
| SPy1697 | hypothetical protein |
| SPy1718 | putative esterase |
| SPy1733 | putative transcription regulator |
| SPy1743 | acetyl-CoA carboxylase alpha subunit |
| SPy1751 | putative trans-2-enoyl-ACP reductase II |
| SPy1795 | putative ABC transporter (periplasmic |
| | binding protein) |
| SPy1796 | hypothetical protein |
| SPy1801 | immunogenic secreted protein precursor |
| | homo log |
| SPy1813 | hypothetical protein |
| SPy1874 | putative glycoprotein endopeptidase |
| SPy1877 | putative glutamine synthetase |
| SPy1882 | putative acid phosphatase |
| SPy1939 | hypothetical protein |
| SPy1959 | hypothetical protein |
| SPy1972 | putative pullulanase |
| SPy1979 | streptokinase A precursor |
| SPy1983 | collagen-like surface protein |
| SPy2000 | surface lipoprotein |
| SPy2001 | transmembrane transport protein |
| SPy2007 | putative laminin adhesion |
| SPy2009 | hypothetical protein |
| SPy2010 | C5A peptidase precursor |
| SPy2018 | M protein type 1 |
| SPy2025 | immunogenic secreted protein precursor |
| SPy2033 | hypothetical protein |
| SPy2037 | peptidylprolyl isomerase |
| SPy2043 | mitogenic factor |
| SPy2066 | putative dipeptidase |
| SPy2209 | hypothetical protein |
| SPyM3_1727 | M protein type 3 |

^{(a)}when the SPy number is not available, the gi- number is indicated.
^{(b)}M5005 SPy number since the protein was present in SF370 but was not annotated.

TABLE 2
GAS antigens reacting equally against tic, pharyngitis and no tic sera

| | GAS | SEQ | | | | |
|---|---|---|---|---|---|---|
| SPy^{(a)} | no. | EiD | Annotation | Tic | Phar^{(b)} | No Tic |
| SPy0269 | 40 | 6&7 | putative surface exclusion protein | 98 | 89 | 93 |
| gi-507127 | N/A | 53 | M protein type 9 | 97 | 87 | 89 |
| SPy2010 | 191 | 37 | C5A peptidase precursor | 92 | 67 | 89 |
| MGAS10270_SPy1784 | N/A | 51 | M protein type 2 | 89 | 94 | 83 |
| SPy1801 | 97 | 24 | immunogenic secreted protein | 89 | 84 | 83 |
| | | | precursor homolog | | | |
| SPy1813 | 380 | 46 | hypothetical protein | 89 | 82 | 94 |
| SPy1979 | 99 | 26 | streptokinase A precursor | 89 | 81 | 94 |
| SPy1361 | 88 | 20 | putative internalin A precursor | 89 | 80 | 91 |
| gi-126660 | N/A | 49 | M protein type 12 | 89 | 66 | 86 |
| SPy0167 | 25 | 4 | streptolysin O precursor | 89 | 63 | 97 |
| SPy0843 | 158 | 32 | hypothetical protein | 87 | 81 | 74 |
| SPy0857 | 208 | 42 | putative peptidoglycan hydrolase | 87 | 76 | 94 |
| gi-4586375 | N/A | 50 | M protein type 23 | 87 | 74 | 83 |
| SPy2025 | 193 | 39 | immunogenic secreted protein | 87 | 59 | 89 |
| | | | precursor | | | |
| SPyM3_1727 | N/A | 52 | M protein type 3 | 85 | 83 | 86 |
| SPy0416 | 57 | 10& 11 | putative cell envelope | 85 | 62 | 83 |
| | | | proteinase | | | |
| SPy2043 | 195 | 40 | mitogenic factor | 82 | 59 | 91 |
| SPy2018 | 192 | 38 | M protein type 1 | 74 | 85 | 63 |
| SPy0019 | 5 | 1 | putative secreted protein | 74 | 49 | 83 |
| SPy1972 | 187 | 34 | putative pullulanase | 70 | 50 | 60 |
| SPy0457 | 63 | 14 | putative cyclophillin-type | 57 | 46 | 34 |
| | | | protein | | | |
| SPy2009 | 190 | 36 | hypothetical protein | 46 | 33 | 26 |
| SPy1733 | 95 | 22 | putative transcription regulator | 43 | 35 | 26 |
| SPy0436 | 60 | 12 | putative exotoxin | 38 | 25 | 31 |
| SPy1007 | 219 | 43 | streptococcal exotoxin I | 30 | 40 | 43 |

Numeric values refer to % of positive sera of each class versus each antigen.
^{(a)}When the SPy number is not available, the gi- number is indicated.
^{(b)}Pharyngitis.

TABLE 3
GAS antigens preferentially reacting against tic and pharyngitis sera

| | GAS | SEQ | | | | |
|---|---|---|---|---|---|---|
| SPy | no. | ID | Annotation | Tic | Phar^{(a)} | No Tic |
| SPy0747 | 143 | 31 | hypothetical protein | 90 | 92 | 51 |
| SPy0031 | 6 | 2 | putative choline binding protein | 64 | 83 | 29 |
| SPy1032 | 75 | 17 | extracellular hyaluronate lyase | 62 | 74 | 29 |
| SPy0159 | 22 | 3 | hypothetical protein | 59 | 69 | 26 |
| SPy0737 | 68 | 16 | putative extracellular matrix | 59 | 60 | 17 |
| | | | binding protein | | | |
| SPy0793 | 473 | 47 | hypothetical protein | 56 | 65 | 22 |
| SPy1037 | 76 | 18 | hypothetical protein | 54 | 52 | 20 |
| SPy0714 | 67 | 5 | putative adhesion protein | 54 | 44 | 17 |
| SPy2000 | 100 | 27 | surface lipoprotein | 52 | 64 | 6 |
| SPy1390 | 89 | 21 | putative protease maturation | 51 | 77 | 9 |
| | | | protein | | | |
| SPy2037 | 103 | 29 | peptidylprolyl isomerase | 51 | 72 | 9 |
| SPy0252 | 36 | 5 | putative sugar transporter sugar | 51 | 69 | 3 |
| | | | binding lipoprotein | | | |
| SPy1882 | 98 | 25 | putative acid phosphatase | 51 | 62 | 9 |
| SPy1983 | 188 | 35 | collagen-like surface protein | 51 | 46 | 23 |
| M5005_SPy0249^{(b)} | 45 | 9 | oligopeptidepermease OppA | 49 | 63 | 20 |
| SPy0287 | 42 | 8 | hypothetical protein | 49 | 60 | 9 |
| SPy0441 | 62 | 13 | hypothetical protein | 43 | 52 | 6 |
| SPy2007 | 101 | 28 | putative laminin adhesion | 38 | 27 | 0 |
| SPy1326 | 165 | 33 | hypothetical protein | 38 | 28 | 9 |
| SPy2066 | 105 | 30 | putative dipeptidase | 33 | 31 | 11 |
| SPy0838 | 477 | 48 | hypothetical protein | 30 | 34 | 4 |

Numeric values refer to % of positive sera of each class versus each antigen. Recognition percentages from No Tic sera were significantly lower (P < 0.05) when compared to those obtained with either Tic or Pharyngitis sera, as established by using the two-tailed χ² test.
^{(a)}Pharyngitis.
^{(b)}M5005 SPy number used since the protein, although present, was not annotated in SF370 M1 strain.

TABLE 4
GAS antigens preferentially reacting against tic sera

| | GAS | SEQ | | | | |
|---|---|---|---|---|---|---|
| | no. | ID | Annotation | Tic | Phar^{(a)} | No Tic |
| SPy0453 | 205 | 41 | metal binding protein | 41 | 25 | 9 |
| | | | of ABC transporter | | | |
| SPy1939 | 277 | 45 | hypothetical protein | 39 | 13 | 11 |
| SPy1795 | 96 | 23 | Putative ABC | 38 | 22 | 3 |
| | | | transporter | | | |
| SPy1054 | 77 | 19 | Putative collagen-like | 36 | 14 | 3 |
| | | | protein | | | |
| SPy1306 | 242 | 44 | maltose/maltodextrin- | 34 | 15 | 0 |
| | | | binding protein | | | |

^{(a)}Pharyngitis.
Numeric values refer to % of positive sera of each class versus each antigen.
Number of tic, pharyngitis and no tic sera positive against each antigen were compared using two-tailed χ² test, resulting in a statistically higher number of positive tic sera as compared to pharyngitis and no tic sera (P < 0.05).

TABLE 5
Sera reacting against multiple tic-preferentially-recognized antigens

| Number | | | | | |
|---|---|---|---|---|---|
| positive | | | P | | P |
| antigens^{(a)} | Tic^{(b)} | Pharyngitis^{(b)} | value^{(c)} | No Tic^{(b)} | value^{(d)} |
| 5 | 23% (14/61) | 5% (11/239) | <0.0001 | 0% (0/35) | 0.0057 |
| at least 4 | 33% (20/61) | 6% (15/239) | <0.0001 | 0% (0/35) | 0.0004 |
| at least 3 | 39% (24/61) | 17% (41/239) | 0.0003 | 3% (1/35) | 0.0002 |

^{(a)}number of antigens reported in Table 4 simultaneously recognized by the same serum.
^{(b)}percentages of positive sera (number of positive sera/total sera tested) simultaneously recognizing the number of antigens indicated in the first column.
^{(c)}P values of Tic versus Pharyngitis calculated with two-tailed χ² test.
^{(d)}P values of Tic versus No Tic calculated with two-tailed χ² test.

### REFERENCES

[1] Cunningham M W. Clin. Microbiol. Rev. 13, 470-511 (2000).

[2] Cunningham M W, Adv. Exp. Med Biol., 609: 29-42 (2008)

[3] Martino D, Giovannoni G, Adv. Neurol., 96:320-335 (2005)

[4] Weiner S G, Normadin P A, Pediatr. Emerg. Care, 23: 20-24 (2007)

[5] Dale R C, Chruch A J, Cardoso F, Goddard E, Cox T C, et al, Annals of Neurology, 40:588-595 (2001)

[6] Swedo S E, Leonard H L, Garvey M, Mittelman B, Allen A J, et al, Am J. Psychiatry 155, 264-271 (1998)

[7] Luo F, Leckman J F, Katsovich L, Findley D, Grantz H, et al. Pediatrics 113, e578-585 (2004).

[8] Murphy M L & Pichichero M E Arch Pediatr Adolesc Med 156, 356-361 (2002).

[9] Kurlan R, Johnson D, Kaplan E L et al, Pediatrics 121: 1188-1197 (2008)

[10] Ferretti J J, McShan W M, Ajdic D, Savic D J, Savic G, Lyon K, Primeaux C, Sezate S, Suvorov A N, Kenton S, Lai H S, Lin S P, Qian Y, Jia H G, Najar F Z, Ren Q, Zhu H, Song L, White J, Yuan X, Clifton S W, Roe B A, McLaughlin R:. Proc Natl Acad Sci USA, 98:4658-4663 (2001)

[11] Cretich, M., Damin F., et al, Biomolecular Engineering 23, 77-88 (2006).

[12] Zhu, H & Snyder, M., Current Opinion in Chemical Biology, 7:55-63 (2003)

[13] Leckman J F, Block M H, King R A, Scahill L, Adv Neurol, 99: 1-16 (2006)

[14] Martin J M, Green M, Barbadora K A, Wald E R, Pediatrics, 114:1212-1219 (2004)

[15] Cardona F, Orefici G, J Pediatr, 138: 71-75 (2001)

[16] Robinson W H, DiGennaro C, Hueber W, Haab B B, Kamachi M, et al, Nat. Med., 8:295-301 (2002)

[17] Garvey M A, Giedd J, Swedo S E, J. Child Neurol, 13:413-423 (1998)

[18] Dale R C, Dev Med Child Neurol, 47: 785-791 (2005)

[19] Lougee L, Perlmutter S J, Nicolson R, Garvey M A, Swedo S E, J. Am Acad child Adolesc Psychiatry, 39: 1120-1126 (2000)

[20] Cunningham M W, Molecular Immunology, 40: 1121-1127 (2004)

[21] Guilherme L, Fae K C, Oshiro S E, Tanaka A C, Pomerantzeff P M, et al, Curr Protein pepti Sci, 8: 39-44 (2007)

[22] Fontan P A, Pancholi V, Nociari M M, Fischetti V A, I Infect Dis, 182:1712-1721 (2000)

[23] Martins T B, Hoffman J L, Augustine N H, Phansalkar A R, Fischetti V A, Int Immunol., 20:445-452 (2008)

[24] Church A J, Dale R C, Lees A J, Giovannoni G, Robertson M, J. Neurol. Neurosurg Psychiatry, 74: 602-607 (2003)

[25] Dale R C, Heyman I, Giovanni G, Church A W J, Br. J. Of Psychiatry, 187:314-319 (2005)

[26] Muller N, Kroll B, Scwarz M J, Riedel M, Straube A, Psychiatry Res 101: 187-193 (2001)

[27] Bronze M S, Dale J B, J. Immunol, 151: 2820-2828 (1993)

[28] Kirvan C A, Swedo S E Heuser J S, Cunningham M W, Nat Med, 9: 914-920 (2003)

[29] Kansy J W, Katsovich L, McIver K S, Pick J, Zabriskie J B, J Neuroimmunol, 181: 165-176 (2006)

[30] Dale R C, Candler P M, Chruch A J, Wait R, Pocock J M, J Neuroimmunol 172: 187-197 (2006)

[31] Harris K, Singer H S, J. Child Neurol, 21: 678-689 (2006)

[32] Motigiani S, Falugi F, Scarselli M, Finco O, Petracca R, Infect Immuno, 70: 368-379 (2002)

[33] Harris J W, Stocker H, Handbook of Mathematics and Computational Science 1: 824 (1998)]

Sequence listing:
Portions that are underlined were not expressed on the basis that
they appeared to be leader sequences or transmembrane domains: -
SEQ ID NO: 1 - SPy0019; gas5
MKKRILSAVLVSGVTLGAATTVGAEDLSTKIAKQDSIISNLTTEQKAAQNQVSALQAQVSSLQSEQDKLTA
RNTELEALSKRFEQEIKALTSQIVARNEKLKNQARSAYKNNETSGYINALLNSKSISDVVNRLVAINRAVS
ANAKLLEQQKADKVSLEEKQAANQTAINTIAANMAMAEENQNTLRTQQANLVAATANLALQLASATEDKAN
LVAQKEAAEKAAAEALAQEQAAKVKAQEQAAQQAASVEAAKSAITPAPQATPAAQSSNAIEPAALTAPAAP
SAGPQTSYDSSNTYPVGQCTWGAKSLAPWAGNNWGNGGQWAYSAQAAGYRTGSTPMVGAIAVWNDGGYGHV
AVVVEVQSASSIRVMESNYSGRQYIADHRGWENPTGVTFIYPH
SEQ ID NO: 2 - SPy0031; gas6
MKKEHRFLVSGVILLGFNGLVPTMPSTLISQQENLVHAAVLGDNYPSKWKKGNGIDSWNMYIRQCTSFAAF
RLSSANGFQLPKGYGNACTWGHIAKNQGYPVNKTPSIGAIAWFDKNAYQSNAAYGHVAWVADIRGDTVTIE
EYNYNAGQGPERYHKRQIPKSQVSGYIHFKDLSSQTSHSYPRQLKHISQASFDPSGTYHFTTRLPVKGQTS
IDSPDLAYYEAGQSVYYDKVVTAGGYTWLSYLSFSGNRRYIPIKEPAQSVVQNDNTKPSIKVGDTVTFPGV
FRVDQLVNNLIVNKELAGGDPTPLNWIDPTPLDETDNQGKVLGDQILRVGEYFIVTGSYKVLKIDQPSNGI
YVQIGSRGTWVNADKANKL
SEQ ID NO: 3 - SPy0159; gas22
MIRKYDRTSTKKKSLNWIWLIIAFFMISSFIGGSSFTESLLDILPAIAIGGTGYAIFRVRSHQKRLAKAKI
AKQLEDLKAKIQLADRKVRLLDTYLADHDDFQYNVLAQQLLPQLSDIKAKAITLKDQLDPQIYRRITKKAN
DVESDITLQLETLQIATTLNPQPLKTPSPNLINKAPELKPYYDNIQTDHQAILAKIQGADNQEELLALHDA
NMRRFEDILTGYLKIKEEPKNYYNAAARLEQAKQAIQQFDEDLDETLRRLNESDLKDFDISLRIMQGATQR
RTTHHQKD
SEQ ID NO: 4 - SPy0167; gas25
MSNKKTFKKYSRVAGLLTAALIIGNLVTANAESNKQNTASTETTTTNEQPKPESSELTTEKAGQKTDDMLN
SNDMIKLAPKEMPLESAEKEEKKSEDKKKSEEDHTEEINDKIYSLNYNELEVLAKNGETIENFVPKEGVKK
ADKFIVIERKKKNINTTPVDISIIDSVTDRTYPAALQLANKGFTENKPDAVVTKRNPQKIHIDLPGMGDKA
TVEVNDPTYANVSTAIDNLVNQWHDNYSGGNTLPARTQYTESMVYSKSQIEAALNVNSKILDGTLGIDFKS
ISKGEKKVMIAAYKQIFYTVSANLPNNPADVFDKSVTFKELQRKGVSNEAPPLFVSNVAYGRTVFVKLETS
SKSNDVEAAFSAALKGTDVKTNGKYSDILENSSFTAVVLGGDAAEHNKVVTKDFDVIRNVIKDNATFSRKN
PAYPISYTSVFLKNNKIAGVNNRTEYVETTSTEYTSGKINLSHQGAYVAQYEILWDEINYDDKGKEVITKR
RWDNNWYSKTSPFSTVIPLGANSRNIRIMARECTGLAWEWWRKVIDERDVKLSKEINVNISGSTLSPYGSI
TYK
SEQ ID NO: 5 - SPy0252; gas36
MNMKKLASLAMLGASVLGLAACGGKSQKEAGASKSDTAKTEITWWAFPVFTQEKAEDGVGTYEKKLIAAFE
KANPEIKVKLETIDFTSGPEKITTAIEAGTAPDVLFDAPGRIIQYGKNGKLADLNDLFTEEFTKDVNNDKL
IQASKAGDTAYMYPISSAPFYMALNKKMLKDAGVLDLVKEGWTTDDFEKVLKALKDKGYNPGSFFANGQGG
DQGPRAFFANLYSSHITDDKVTKYTTDDANSIKAMTKISNWIKDGLMMNGSQYDGSADIQNFANGQTSFTI
LWAPAQPGIQAKLLEASKVDYLEIPFPSDDGKPELEYLVNGFAVFNNKDEQKVAASKTFIQFIADDKEWGP
KNVVRTGAFPVRTSYGDLYKDKRMEKIAEWTKFYSPYYNTIDGFAEMRTLWFPMVQAVSNGDEKPEDALKA
FTEKANKTIKKTQ
SEQ ID NO: 6 - SPy0269; gas40
MDLEQTKPNQVKQKIALTSTIALLSASVGVSHQVKADDRASGETKASNTHDDSLPKPETIQEAKATIDAVE
KTLSQQKAELTELATALTKTTAEINHLKEQQDNEQKALTSAQEIYTNTLASSEETLLAQGAEHQRELTATE
TELHNAQADQHSKETALSEQKASISAETTRAQDLVEQVKTSEQNIAKLNAMISNPDAITKAAQTANDNTKA
LSSELEKAKADLENQKAKVKKQLTEELAAQKAALAEKEAELSRLKSSAPSTQDSIVGNNTMKAPQGYPLEE
LKKLEASGYIGSASYNNYYKEHADQIIAKASPGNQLNQYQDIPADRNRFVDPDNLTPEVQNELAQFAAHMI
NSVRRQLGLPPVTVTAGSQEFARLLSTSYKKTHGNTRPSFVYGQPGVSGHYGVGPHDKTIIEDSAGASGLI
RNDDNMYENIGAFNDVHTVNGIKRGIYDSIKYMLFTDHLHGNTYGHAINFLRVDKHNPNAPVYLGFSTSNV
GSLNEHFVMFPESNIANHQRFNKTPIKAVGSTKDYAQRVGTVSDTIAAIKGKVSSLENRLSAIHQEADIMA
AQAKVSQLQGKLASTLKQSDSLNLQVRQLNDTKGSLRTELLAAKAKQAQLEATRDQSLAKLASLKAALHQT
EALAEQAAARVTALVAKKAHLQYLRDFKLNPNRLQVIRERIDNTKQDLAKTTSSLLNAQEALAALQAKQSS
LEATIATTEHQLTLLKTLANEKEYRHLDEDIATVPDLQVAPPLTGVKPLSYSKIDTTPLVQEMVKETKQLL
EASARLAAENTSLVAEALVGQTSEMVASNAIVSKITSSITQPSSKTSYGSGSSTTSNLISDVDESTQRALK
AGVVMLAAVGLTGFRFRKESK
SEQ ID NO: 7 - SPy0269; gas40N
SVGVSHQVKADDRASGETKASNTHDDSLPKPETIQEAKATIDAVEKTLSQQKAELTELATALTKTTAEINH
LKEQQDNEQKALTSAQEIYTNTLASSEETLLAQGAEHQRELTATETELHNAQADQHSKETALSEQKASISA
ETTRAQDLVEQVKTSEQNIAKLNAMISNPDAITKAAQTANDNTKALSSELEKAKADLENQKAKVKKQLTEE
LAAQKAALAEKEAELSRLKSSAPSTQDSIVGNNTMKAPQGYPLEELKKLEASGYIGSASYNNYYKEHADQI
IAKASPGNQLNQYQ
SEQ ID NO: 8 - SPy0287; gas42
MTKEKLVAFSQAHAEPAWLQERRLAALEAIPNLELPTIERVKFHRWNLGDGTLTENESLASVPDFIAIGDN
PKLVQVGTQTVLEQLPMALIDKGVVFSDFYTALEEIPEVIEAHFGQALAFDEDKLAAYHTAYFNSAAVLYV
PDHLEITTPIEAIFLQDSDSDVPFNKHVLVIAGKESKFTYLERFESIGNATQKISANISVEVIAQAGSQIK
FSAIDRLGPSVTTYISRRGRLEKDANIDWALAVMNEGNVIADFDSDLIGQGSQADLKVVAASSGRQVQGID
TRVTNYGQRTVGHILQHGVILERGTLTFNGIGHILKDAKGADAQQESRVLMLSDQARADANPILLIDENEV
TAGHAASIGQVDPEDMYYLMSRGLDQETAERLVIRGFLGAVIAEIPIPSVRQEIIKVLDEKLLNR
SEQ ID NO: 9 - M5005_SPy0249; gas45
MKKSKWLAAVSVAILSVSALAACGNKNASGGSEATKTYKYVFVNDPKSLDYILTNGGGTTDVITQMVDGL
LENDEYGNLVPSLAKDWKVSKDGLTYTYTLRDGVSWYTADGEEYAPVTAEDFVTGLKHAVDDKSDALYVV
EDSIKNLKAYQNGEVDFKEVGVKALDDKTVQYTLNKPESYWNSKTTYSVLFPVNAKFLKSKGKDFGTTDP
SSILVNGAYFLSAFTSKSSMEFHKNENYWDAKNVGIESVKLTYSDGSDPGSFYKNFDKGEFSVARLYPND
PTYKSAKKNYADNITYGMLTGDIRHLTWNLNRTSFKNTKKDPAQQDAGKKALNNKDFRQAIQFAFDRASF
QAQTAGQDAKTKALRNMLVPPTFVTIGESDFGSEVEKEMAKLGDEWKDVNLADAQDGFYNPEKAKAEFAK
AKEALTAEGVTFPVQLDYPVDQANAATVQEAQSFKQSVEASLGKENVIVNVLETETSTHEAQGFYAETPE
QQDYDIISSWWGPDYQDPRTYLDIMSPVGGGSVIQKLGIKAGQNKDVVAAAGLDTYQTLLDEAAAITDDN
DARYKAYAKAQAYLTDNAVDIPVVALGGTPRVTKAVPFSGGFSWAGSKGPLAYKGMKLQDKPVTVKQYEK
AKEKWMKAKAKSNAKYAEKLADHVEK
SEQ ID NO: 10 - SPy0416; gas57
MEKKQRFSLRKYKSGTFSVLIGSVFLVMTTTVAADELSTMSEPTITNHAQQQAQHLTNTELSSAESKSQDT
SQITLKTNREKEQSQDLVSEPTTTELADTDAASMANTGSDATQKSASLPPVNTDVHDWVKTKGAWDKGYKG
QGKVVAVIDTGIDPAHQSMRISDVSTAKVKSKEDMLARQKAAGINYGSWINDKVVFAHNYVENSDNIKENQ
FEDFDEDWENFEFDAEAEPKAIKKHKIYRPQSTQAPKETVIKTEETDGSHDIDWTQTDDDTKYESHGMHVT
GIVAGNSKEAAATGERFLGIAPEAQVMFMRVFANDIMGSAESLFIKAIEDAVALGADVINLSLGTANGAQL
SGSKPLMEAIEKAKKAGVSVVVAAGNERVYGSDHDDPLATNPDYGLVGSPSTGRTPTSVAAINSKWVIQRL
MTVKELENRADLNHGKAIYSESVDFKDIKDSLGYDKSHQFAYVKESTDAGYNAQDVKGKIALIERDPNKTY
DEMIALAKKHGALGVLIFNNKPGQSNRSMRLTANGMGIPSAFISHEFGKAMSQLNGNGTGSLEFDSVVSKA
PSQKGNEMNHFSNWGLTSDGYLKPDITAPGGDIYSTYNDNHYGSQTGTSMASPQIAGASLLVKQYLEKTQP
NLPKEKIADIVKNLLMSNAQIHVNPETKTTTSPRQQGAGLLNIDGAVTSGLYVTGKDNYGSISLGNITDTM
TFDVTVHNLSNKDKTLRYDTELLTDHVDPQKGRFTLTSHSLKTYQGGEVTVPANGKVTVRVTMDVSQFTKE
LTKQMPNGYYLEGFVRFRDSQDDQLNRVNIPFVGFKGQFENLAVAEESIYRLKSQGKTGFYFDESGPKDDI
YVGKHFTGLVTLGSETNVSTKTISDNGLHTLGTFKNADGKFILEKNAQGNPVLAISPNGDNNQDFAAFKGV
FLRKYQGLKASVYHASDKEHKNPLWVSPESFKGDKNFNSDIRFAKSTTLLGTAFSGKSLTGAELPDGHYHY
VVSYYPDVVGAKRQEMTFDMILDRQKPVLSQATFDPETNRFKPEPLKDRGLAGVRKDSVFYLERKDNKPYT
VTINDSYKYVSVEDNKTFVERQADGSFILPLDKAKLGDFYYMVEDFAGNVAIAKLGDHLPQTLGKTPIKLK
LTDGNYQTKETLKDNLEMTQSDTGLVTNQAQLAVVHRNQPQSQLTKMNQDFFISPNEDGNKDFVAFKGLKN
NVYNDLTVNVYAKDDHQKQTPIWSSQAGASVSAIESTAWYGITARGSKVMPGDYQYVVTYRDEHGKEHQKQ
YTISVNDKKPMITQGRFDTINGVDHFTPDKTKALDSSGIVREEVFYLAKKNGRKFDVTEGKDGITVSDNKV
YIPKNPDGSYTISKRDGVTLSDYYYLVEDRAGNVSFATLRDLKAVGKDKAVVNFGLDLPVPEDKQIVNFTY
LVRDADGKPIENLEYYNNSGNSLILPYGKYTVELLTYDTNAAKLESDKIVSFTLSADNNFQQVTFKITMLA
TSQITAHFDHLLPEGSRVSLKTAQDQLIPLEQSLYVPKAYGKTVQEGTYEVVVSLPKGYRIEGNTKVNTLP
NEVHELSLRLVKVGDASDSTGDHKVMSKNNSQALTASATPTKSTTSATAKALPSTGEKMGLKLRIVGLVLL
GLTCVFSRKKSTKD
SEQ ID NO: 11 - SPy0416; gas57N
ADELSTMSEPTITNHAQQQAQHLTNTELSSAESKSQDTSQITLKTNREKEQSQDLVSEPTTTELADTDAAS
MANTGSDATQKSASLPPVNTDVHDWVKTKGAWDKGYKGQGKVVAVIDTGIDPAHQSMRISDVSTAKVKSKE
DMLARQKAAGINYGSWINDKVVFAHNYVENSDNIKENQFEDFDEDWENFEFDAEAEPKAIKKHKIYRPQST
QAPKETVIKTEETDGSHDIDWTQTDDDTKYESHGMHVTGIVAGNSKEAAATGERFLGIAPEAQVMFMRVFA
NDIMGSAESLFIKAIEDAVALGADVINLSLGTANGAQLSGSKPLMEAIEKAKKAGVSVVVAAGNERVYGSD
HDDPLATNPDYGLVGSPSTGRTPTSVAAINSKWVIQRLMTVKELENRADLNHGKAIYSESVDFKDIKDSLG
YDKSHQFAYVKESTDAGYNAQDVKGKIALIERDPNKTYDEMIALAKKHGALGVLIFNNKPGQSNRSMRLTA
NGMGIPSAFISHEFGKAMSQLNGNGTGSLEFDSVVSKAPSQKGNEMNHFSNWGLTSDGYLKPDITAPGGDI
YSTYNDNHYGSQTGTSMASPQIAGASLLVKQYLEKTQPNLPKEKIADIVKNLLMSNAQIHVNPETKTTTSP
RQQGAGLLNIDGAVTSGLYVTGKDNYGSISLGNITDTMTFDVTVHNLSNKDKTLRYDTELLTDHVDPQKGR
FTLTSHSLKTYQGGEVTVPANGKVTVRVTMDVSQFTKELTKQMPNGYYLEGFVRFRDSQDDQLNRVNIPFV
GFKGQFENLAVAEESIYRLKSQGKTGFYFDESGPKDDIYVGKHFTGLVTLGSETNVSTKTISDNGLHTLGT
FKNADGKFILEKN
SEQ ID NO: 12 - SPy0436; gas60
MKRIIKTIILVIIIFHGYGSVKSDSENIKDVKLQLNYAYEIIPVDYTNCNIDYLTTHDFYIDISSYKKKNF
SVDSEVESYITTKFTKNQKVNIFGLPYIFTRYDVYYIYGGVTPSVNSNSENSKIVGNLLIDGVQQKTLINP
IKIDKPIFTIQEFDFKIRQYLMQTYKIYDPNSPYIKGQLEIAINGNKHESFNLYDATSSSTRSDIFKKYKD
NKTINMKDFSHFDIYLWTK
SEQ ID NO: 13 - SPy0441; gas62
MKLAVLGTGMIVKEVLPVLQKIDGIDLVAILSTVRSLTTAKDLAKAHHMPLATSKYEAILGNEEIDTVYIG
LPNHLHFAYAKEALLAGKHVICEKPFTMTAGELDELVVIARKRKLILLEAITNQYLSNMTFIKEHLDQLGD
IKIVECNYSQYSSRYDAFKRGDIAPAFNPKMGGGALRDLNIYNIHFVVGLFGRPKTVQYLANVEKGIDTSG
MLVMDYEQFKVVCIGAKDCTAEIKSTIQGNKGSLAVLGATNTLPQVQLSLHGHEPQVINHNKHDHRMYEEF
VAFRDMIDQRDFEKVNQALEHSRAVMAVLERAVHS
SEQ ID NO: 14 - SPy0457; gas63
MKKLLSLSLVAISLLNLSACESVDRAIKGDKYIDEKTAKEESEAASKAYEESIQKALKADASQFPQLTKEV
GKEEAKVVMRTSQGDITLKLFPKYAPLAVENFLTHAKKGYYDNLTFHRVINDFMIQSGDPKGDGTGGESIW
KGKDPKKDAGNGFVNEISPFLYHIRGALAMANAGANTNGSQFYINQNKKNQSKGLSSTNYPKEIISAYEHG
GNPSLDGGYTVFGQVIDGMDVVDKIAATSINQNDKPEQDITITSIDIVKDYRFKN
SEQ ID NO: 15 - SPy0714; gas67
MKKKILLMMSLISVFFAWQLTQAKQVLAEGKVKVVTTFYPVYEFTKGVIGNDGDVFMLMKAGTEPHDFEPS
TKDIKKIQDADAFVYMDDNMETWVSDVKKSLTSKKVTIVKGTGNMLLVAGAGHDHPHEDADKKHEHNKHSE
EGHNHAFDPHVWLSPYRSITVVENIRDSLSKAYPEKAENFKANAATYIEKLKELDKDYTAALSDAKQKSFV
TQHAAFGYMALDYGLNQISINGVTPDAEPSAKRIATLSKYVKKYGIKYIYFEENASSKVAKTLAKEAGVKA
AVLSPLEGLTEKEMKAGQDYFTVMRKNLETLRLTTDVAGKEILPEKDTTKTVYNGYFKDKEVKDRQLSDWS
GSWQSVYPYLQDGTLDQVWDYKAKKSKGKMTAAEYKDYYTTGYKTDVEQIKINGKKKTMTFVRNGEKKTFT
YTYAGKEILTYPKGNRGVRFMFEAKEADAGEFKYVQFSDHAIAPEKAKHFHLYWGGDSQEKLHKELEHWPT
YYGSDLSGREIAQEINAH
SEQ ID NO: 16 - SPy0737; gas68
MRKVKKVFVSSCMLLTVGLGVAVPTGFSQSNGVMVVKAAEVPATDLSRQASDSERVDESSLLQKENLSVDS
FKLENLNGWEAENDTAGNLGKFKDPDSSGYQNILTSSGKNISVAVAPKGSGKMNIKVTKRSNFQGGYYVGG
LRTQTPVLKLNDVYRYSFTTKKLSGNSSEFKTRVKPVESNNKLGKELVIRVDNKNVSTKHDWLPDISDGTH
TVDFTGLDKKLSVAFRFSPRQTSNVVYEFSNINIKNISPASVPAIPSKVLEGTSVLSGTAISSGDTLEKRK
SFDGDILRVYKDSKIIARTVIKGNKWDVKLSKPLIAGEKLDFEILHPRSQNVSKKISKQVEAKPFDPASYK
EKVIAKLKPVYEATSEKITNDAWLDENAKDLQKQKLEEQYISGKVAISEAGTKQEAIDAAYNKYSSQTDPD
SLPSQYKQGNKENEQEKGRQDLIQTRDLTLKAIQEDKWLTEQEKTIQKEEALKAFETGIESVNQTVSLEQL
KQRLIVYKASEKDSEKKEYPESIPNQHIPGKEKEVKAAKQEELKKLHDTTLEKINQDKWLTPDQQAEQLKQ
AEVTFKKGQEAIKSAQTLTQLETDLADYVSENEGKGNSIPDKYKSGNKDDLVNKAEVKLKEAHEATKQAIE
KDPWLSPEQKKAQKEKAKARLDEGLKALKAADSLEILKVTEEAFVDKEKNPDSIPNQHKAGTADQARKQAL
DSLDKEVQKELESIDNDNTLTTDEKAAAKKKVNDAYDVAKQTAMEANSYEDLTTIKDEFLSNLPHKQGTPL
KDQQSDAIAELEKKQQEIEKAIEGDKTLPRDEKEKQIADSKERLKSDTQKVKDAKNADAIKKAFEEGKVNI
PQAHIPGDLNKDKEKLLAELKQKADDTEKAIDVDKTLTEDEKKEQKVKTKAELEKAKTDVKNTQTREELDK
KVPELKKAIEDTHVKGNLEGVKNKAIEDLKKAHTETVAKINGDDTLDKATKEAQVKEADKALAAGKDAITK
ADDADKVSTAVTEHTPKIKAAHKTGDLKKAQVDANTALDKAAEKERGEINKDATLTTEDKAKQLKEVETAL
TKAKDNVKAAKTADAINDARDKGVATIDAVHKAGQDLGARKSGQVAKLEEAAKATKDKISADPTLTSKEKE
EQSKAVDAELKKAIEAVNAADTADKVDDALGEGVTDIKNQHKSGDSIDARREAHGKELDRVAQETKGAIEK
DPTLTTEEKAKQVKDVDAAKERGMAKLNEAKDADALDKAYGEGVTDIKNQHKSGDPVDARRGLHNKSIDEV
AQATKDAITADTTLTEAEKETQRGNVDKEATKAKEELAKAKDADALDKAYGDGVTSIKNQHKSGKGLDVRK
DEHKKALEAVAKRVTAEIEADPTLTPEVREQQKAEVQKELELATDKIAEAKDADEADKAYGDGVTAIENAH
VIGKGIEARKDLAKKDLAEAAAKTKALIIEDKTLTDDQRKEQLLGVDTEYAKGIENIDAAKDAAGVDKAYS
DGVRDILAQYKEGQNLNDRRNAAKEFLLKEADKVTKLINDDPTLTHDQKVDQINKVEQAKLDAIKSVDDAQ
TADAINDALGKGIENINNQYQHGDGVDVRKATAKGDLEKEAAKVKALIAKDPTLTQADKDKQTAAVDAAKN
TAIAAVDKATTTEGINQELGKGITAINKAYRPGEGVKARKEAAKADLEKEAAKVKALITNDPTLTKADKAK
QTEAVAKALKAAIAAVDKATTAEGINQELGKGITAINKAYRPGEGVKARKEAAKADLEREAAKVREAIAND
PTLTKADKAKQTEAVAKALKAAIAAVDKATTAEGINQELGKGITAINKAYRPGEGVEAHKEAAKANLEKVA
KETKALISGDRYLSETEKAVQKQAVEQALAKALGQVEAAKTVEAVKLAENLGTVAIRSAYVAGLAKDTDQA
TAALNEAKQAAIEALKQAAAETLAKITTDAKLTEAQKAEQSENVSLALKTAIATVRSAQSIASVKEAKDKG
ITAIRAAYVPNKAVAKSSSANHLPKSGDANSIVLVGLGVMSLLLGMVLYSKKKESKD
SEQ ID NO: 17 - SPy1032; gas75
MNTYFCTHHKQLLLYSNLFLSFAMMGQGTAIYADTLTSNSEPNNTYFQTQTLTTTDSEKKVVQPQQKDYYT
ELLDQWNSIIAGNDAYDKTNPDMVTFHNKAEKDAQNIIKSYQGPDHENRTYLWEHAKDYSASANITKTYRN
IEKIAKQITNPESCYYQDSKAIAIVKDGMAFMYEHAYNLDRENHQTTGKENKENWWVYEIGTPRAINNTLS
LMYPYFTQEEILKYTAPIEKFVPDPTRFRVRAANFSPFEANSGNLIDMGRVKLISGILRKDDLEISDTIKA
IEKVFTLVDEGNGFYQDGSLIDHVVTNAQSPLYKKGIAYTGAYGNVLIDGLSQLIPIIQKTKSPIKADKMA
TIYHWINHSFFPIIVRGEMMDMTRGRSISRFNAQSHVAGIEALRAILRIADMSEEPHRLALKTRIKTLVTQ
GNAFYNVYDNLKTYHDIKLMKELLSDTSVPVQKLDSYVASFNSMDKLALYNNKHDFAFGLSMFSNRTQNYE
AMNNENLHGWFTSDGMFYLYNNDLGHYSENYWATVNPYRLPGTTETEQKPLEGTPENIKTNYQQVGMTGLS
DDAFVASKKLNNTSALAAMTFTNWNKSLTLNKGWFILGNKIIFVGSNIKNQSSHKAYTTIEQRKENQKYPY
CSYVNNQPVDLNNQLVDFTNTKSIFLESDDPAQNIGYYFFKPTTLSISKALQTGKWQNIKADDKSPEAIKE
VSNTFITIMQNHTQDGDRYAYMMLPNMTRQEFETYISKLDIDLLENNDKLAAVYDHDSQQMHVIHYGKKAT
MFSNHNLSHQGFYSFPHPVRQNQQ
SEQ ID NO: 18 - SPy1037; gas76
MKRFLNSRPWLGMVSVFFAILLFLTAASSNHNNSSSQIYSPIETYTHSLKDVPIDMKYDSDKYFISGYSYG
AEVYLTSTNRIKLDSEVNNDTRNFKIVADLTHSHPGTVSVNLRVENLPSGVTATVSPDKISVTIGKKESKV
FPVRGSVDAKQIANGYEISKIETGVNKVEVTSDESTIALIDHVVAKLPDDQVLDRNYSSRVTLQAVSADGT
ILASAIDPAKTNLSVAVKKITKSVPIRVEAVGMMDDSLSDIQYKLSKQTAVISGSREVLEDIDEIIAEVNI
SDVTKNTSKTVSLSSSQVSIEPSVVTVQLTTTKK
SEQ ID NO: 19 - SPy1054; gas77
MLTFGGASAVKAEENEKVREQEKLIQQLSEKLVEINDLQTLNGDKESIQSLVDYLTRRGKLEEEWMEYLNS
GIQRKLFVGPKGPAGEKGEQGPTGKQGERGETGPAGPRGDKGETGDKGAQGPVGPAGKDGQNGKDGLPGKD
GKDGQNGKDGLPGKDGKDGQDGKDGLPGKDGKDGQNGKDGLPGKDGQPGKPAPKTPEVPQNPDTAPHTPKT
PRIPGQSKDVTPAPQNPSNRGLNKPQTQGGNQLAKTPAAHDTHRQLPATGETTNPFFTAAAVAIMTTAGVV
AVAKRQENN
SEQ ID NO: 20 - SPy1361; gas88
MKTKKVIILVGLLLSSQLTLIACQSRGNGTYPIKTKQSRKGMTSNKIKPIKKSKKTNKTHKGVAGVDFPTD
DGFILTKDSKILSKTDQGIVVDHDGHSHFIFYADLKGSPFEYLIPKGASLAKPAVAQRAASQGTSKVADPH
HHYEFNPADIVAEDALGYTVRHDDHFHYILKSSLSGQTQAQAKQVATRLPQTSSLVSTATANGIPGLHFPT
SDGFQFNGQGIVGVTKDSILVDHDGHLHPISFADLRQGGWAHVADQYDPAKKAEKPAETHQTPELSEREKE
YQEKLAYLAEKLGIDPSTIKRVETQDGKLGLEYPHHDHAHVLMLSDIEIGKDIPDPHAIEHARELEKHKVG
MDTLRALGFDEEVILDIVRTHDAPTPFPSNEKDPNMMKEWLATVIKLDLGSRKDPLQRKGLSLLPNLETLG
IGFTPIKDISPVLQFKKLKQLLMTKTGVTDYRFLDNMPQLEGIDISQNNLKDISFLSKYKNLTLVAAADNG
IEDIRPLGQLPNLKFLVLSNNKISDLSPLASLHQLQELHIDNNQITDLSPVSHKESLTVVDLSRNADVDLA
TLQAPKLETLMVNDTKVSHLDFLKNNPNLSSLSINRAQLQSLEGIEASSVIVRVEAEGNQIKSLVLKDKQG
SLTFLDVTGNQLTSLEGVNNFTALDILSVSKNQLTNVNLSKPNKTVTNIDISHNNISLADLKLNEQHIPEA
IAKNFPAVYEGSMVGNGTAEEKAAMATKAKESAQEASESHDYNHNHTYEDEEGHAHEHRDKDDHDHEHEDE
NEAKDEQNHAD
SEQ ID NO: 21 - SPy1390; gas89
MKNSNKLIASVVTLASVMALAACQSTNDNTKVISMKGDTISVSDFYNETKNTEVSQKAMLNLVISRVFEAQ
YGDKVSKKEVEKAYHKTAEQYGASFSAALAQSSLTPETFKRQIRSSKLVEYAVKEAAKKELTTQEYKKAYE
SYTPTMAVEMITLDNEETAKSVLEELKAEGADFTAIAKEKTTTPEKKVTYKFDSGATNVPTDVVKAASSLN
EGGISDVISVLDPTSYQKKFYIVKVTKKAEKKSDWQEYKKRLKAIIIAEKSKDMNFQNKVIANALDKANVK
IKDKAFANILAQYANLGQKTKAASESSTTSESSKAAEENPSESEQTQTSSAEEPTETEAQTQEPAAQ
SEQ ID NO: 22 - SPy1733; gas95
MKIGKKIVLMFTAIVLTTVLALGVYLTSAYTFSTGELSKTFKDFSTSSNKSDAIKQTRAFSILLMGVDTGS
SERASKWEGNSDSMILVTVNPKTKKTTMTSLERDTLTTLSGPKNNEMNGVEAKLNAAYAAGGAQMAIMTVQ
DLLNITIDNYVQINMQGLIDLVNAVGGITVTNEFDFPISIAENEPEYQATVAPGTHKINGEQALVYARMRY
DDPEGDYGRQKRQREVIQKVLKKILALDSISSYRKILSAVSSNMQTNIEISSRTIPSLLGYRDALRTIKTY
QLKGEDATLSDGGSYQIVTSNHLLEIQNRIRTELGLHKVNQLKTNATVYENLYGSTKSQTVNNNYDSSGQA
PSYSDSHSSYANYSSGVDTGQSASTDQDSTASSHRPATPSSSSDALAADESSSSGSGSLVPPANINPQT
SEQ ID NO: 23 - SPy1795; gas96
MIKRCKGIGLALMAFFLVACVNQHPKTAKETEQQRIVATSVAVVDICDRLNLDLVGVCDSKLYTLPKRYDA
VKRVGLPMNPDIELIASLKPTWILSPNSLQEDLEPKYQKLDTEYGFLNLRSVEGMYQSIDDLGNLFQRQQE
AKELRQQYQDYYRAFQAKRKGKKKPKVLILMGLPGSYLVATNQSYVGNLLDLAGGENVYQSDEKEFLSANP
EDMLAKEPDLILRTAHAIPDKVKVMFDKEFAENDIWKHFTAVKEGKVYDLDNTLFGMSAKLNYPEALDTLT
QLFDHVGDHP
SEQ ID NO: 24 - SPy1801; gas97
MNKNKLLRVAMLLSLLAPTAESMTVLAQDVMLETHKATTNETSDSSSKEENNKNAAPTTSDKTDQGPLDAS
AETNSNSLVNADDKKRSDSSQSAIGSSDNKAEAENQVDDKSTDHSKSTDHSKPTDQPKPSPSKVDTAPASS
LSKQLPEARTPIQSLSPYVSDLDLSEIDIPSVNTYAAYVEHWSGKNAYTHHLLSRRYGIKADQIDSYLKST
GIAYDSTRINGEKLLQWEKKSGLDVRAIVAIAMSESSLGTQGIATLLGANMFGYAAFDLDPTQASKFNDDS
AIVKMTQDTIIKNKNSNFALQDLKAAKFSRGQLNFASDGGVYFTDTTGSGKRRAQIMEDLDKWIDDHGGTP
AIPAELKVQSSASFASVPAGYKLSKSYDVLGYQASSYAWGQCTWYVYNRAKELGYQFDPFMGNGGDWKYKV
GYALSKTPKVGYAISFAPGQAGADGTYGHVSIVEDVRKDGSILISESNCIGLGKISYRTFTAQQAEQLTYV
IGKSKN
SEQ ID NO: 25 - SPy1882; gas98
MKSKKVVSVISLTLSLFLVTGCAKVDNNKSVNLKPATKQTYNSYSDDQLRSRENTMSVLWYQRAAETQALY
LQGYQLATDRLKEQLNKPTDKPYSIVLDIDETVLDNSPYQAKNVLEGTGFTPESWDYWVQKKEAKPVAGAK
DFLQFADQNGVQIYYISDRSTTQVDATMENLQKEGIPVQGRDHLLFLEKGVKSKESRRQKVKETTNVTMLF
GDNLLDFADFSKKSQEDRTALLSDLQEEFGRRFIIFPNPMYGSWEGAIYKGEKLDVLKQLEERRKSLKSFK
SEQ ID NO: 26 - SPy1979; gas99
MKNYLSIGVIALLFALTFGTVKSVQAIAGYGWLPDRPPINNSQLVVSMAGIVEGTDKKVFINFFEIDLTSQ
PAHGGKTEQGLSPKSKPFATDNGAMPHKLEKADLLKAIQKQLIANVHSNDGYFEVIDFASDATITDRNGKV
YFADKDGSVTLPTQPVQEFLLKGHVRVRPYKEKPVQNQAKSVDVEYTVQFTPLNPDDDFRpGLKDTKLLKT
LAIGDTITSQELLAQAQSILNKTHPGYTIYERDSSIVTHDNDIFRTILPMDQEFTYHVKNREQAYEINPKT
GIKEKTNNTDLVSEKYYVLKQGEKPYDPFDRSHLKLFTIKYVDVNTNELLKSEQLLTASERNLDFRDLYDP
RDKAKLLYNNLDAFDIMDYTLTGKVEDNHDKNNRVVTVYMGKRPKGAKGSYHLAYDKDLYTEEERKAYSYL
RDTGTPIPDNPKDK
SEQ ID NO: 27 - SPy2000; gas100
MSKYLKYFSIITLFLTGLILVACQQQKPQTKERQRKQRPKDELVVSMGAKLPHEFDPKDRYGVHNEGNITH
STLLKRSPELDIKGELAKTYHLSEDGLTWSFDLHDDFKFSNGEPVTADDVKFTYDMLKADGKAWDLTFIKN
VEVVGKNQVNIHLTEAHSTFTAQLTEIPIVPKKHYNDKYKSNPIGSGPYMVKEYKAGEQAIFVRNPYWHGK
KPYFKKWTWVLLDENTALAALESGDVDMIYATPELADKKVKGTRLLDIPSNDVRGLSLPYVKKGVITDSPD
GYPVGNDVTSDPAIRKALTIGLNRQKVLDTVLNGYGKPAYSIIDKTPFWNPKTAIKDNKVAKAKQLLTKAG
WKEQADGSRKKGDLDAAFDLYYPTNDQLRANLAVEVAEQAKALGITIKLKASNWDEMATKSHDSALLYAGG
RHHAQQFYESHHPSLAGKGWTNITFYNNPTVTKYLDKAMTSSDLDKANEYWKLAQWDGKTGASTLGDLPNV
WLVSLNHTYIGDKRINVGKQGVHSHGHDWSLLTNIAEWTWDESTK
SEQ ID NO: 28 - SPy2007; gas101
MKKGFFLMAMVVSLVMIAGCDKSANPKQPTQGMSVVTSFYPMYAMTKEVSGDLNDVRMIQSGAGIHSFEPS
VNDVAAIYDADLFVYHSHTLEAWARDLDPNLKKSKVDVFEASKPLTLDRVKGLEDMEVTQGIDPATLYDPH
TWTDPVLAGEEAVNIAKELGRLDPKHKDSYTKNAKAFKKEAEQLTEEYTQKFKKVRSKTFVTQHTAFSyLA
KRFGLKQLGISGISPEQEPSPRQLKEIQDFVKEYNVKTIFAEDNVNPKIAHAIAKSTGAKVKTLSPLEAAP
SGNKTYLENLRANLEVLYQQLK
SEQ ID NO: 29 - SPy2037; gas103
MKQMNKLITGVVTLATVVTLSACQSSHNNTKLVSMKGDTITVSDFYNETKNTELAQKAMLSLVISRVFETQ
YANKVSDKEVEKAYKQTADQYGTSFKTVLAQSGLTPETYKKQIRLTKLVEYAVKEQAKNETISKKDYRQAY
DAYTPTMTAEIMQFEKEEDAKAALEAVKAEGADFAAIAKEKTTAADKKTTYTFDSGETTLPAEVVRAASGL
KEGNRSEIITALDPATSKRTYHIIKVTKKATKKADWKAYQKRLKDIIVTGKLKDPDFQNKVIAKALDKANV
KIKDKAFANILAQFAKPNQKQPAQK
SEQ ID NO: 30 - SPy2066; gas105
MINKKISLGVLSILTAFSLQSVSYACTGFIIGKDLTKDGSLLYGRTEDLEPHHNKNFIVRLAKDNPAGEKW
KDLSNGFEYPLPEHSYRYSAIPDVTPNKGVYDEAGFNEFGVSMSATVSASANDAIQKIDPYVKNGLAESSM
TSVILPSVKTAREGVALIAKIVTEKGAAEGNIVTLADKDGIWYMEILSGHQYVAIKFPDDKYAVFPNTFYL
GHVDFNDKENTIASEDVEKVAKKAKSYTEVDGKFHIAKSYNPPLNDANRSRSFSGIKSLDPDSKVTYKDSN
YELLQSTDKTFSLEDAMKLQRNRFEGLDLKPLDQMALDGKGKPKSKKAVKGYAYPISNPNVMEAHIFQLKK
DIPAELGGVMWLSIGSPRNAPYLPYLGNISRTYEAYQEKSTQYNDKSWYWTVSHINDLVAAHPKPFGTKVI
DEMKGLEKTWIAEQDKSTKEISDLVVSDPKAAQEKADKISLDRAEKTFKRLKAIEAKLVKEKPKNKKGLNR
S
SEQ ID NO: 31 - SPy0747; gas143
MINKKCIIPVSLLTLAITLTSVEEVTSRQNLTYANEIVTQRPKRESVISDKSNFPVISPYLASVDFGERKT
PLPTPDKGVKVTTEQSIAQVRKGPEERPYTVTGKITSVINGWGGYGFYIQDSEGIGLYVYPQKDLGYSKGD
IVQLTGTLTRFKGDLQLQQVTAHKKLELSFPTSVKEAVISELETTTPSTLVKLSHVTVGELSTDQYNNTSF
LVRDDSGKSIVVHIDHRTGVKGADVVTKISQGDLINLTAILSIVDGQLQLRPFSLEQLEVVKKVTSSNSDA
SSRNIVKIGEIQGASHTSPLLKKAVTVEQVVVTYLDDSTHFYVQDLNGDGDLATSDGIRVFAKNAKVQVGD
VLTISGEVEEFFGRGYEERKQTDLTITQIVAKAVTKTGTAQVPSPLVLGKDRIAPANIIDNDGLRVFDPEE
DAIDYWESMEGMLVAVDDAKILGPMKNKEIYVLPGSSTRPLNNSGGVLLPANSYNTDVIPVLFKKGKQIIK
AGDSYKGRLAGPVSYSYGNYKVFVDDSKNMPSLMDGHLKPEKTNLQKDLSKLSIASYNIENFSANPSSTKD
EKVKRIAESFIHDLNAPDIIGLIEVQDNNGPTDDGTTDATQSAQRLIDAIKKLGGPTYRYVDIAPENNVDG
GQPGGNIRTGFLYQPERVSLSDKPKGGARDALTWVNGELNLSVGRIDPTNAAWKDVRKSLAAEFIFQGRKV
VVVANHLNSKRGDNALYGCVQPVTFKSEQRRHVLANMLAQFAKEGAKHQANIVMLGDFNDFEFTKTIQLIE
EGDMVNLVSRHDISDRYSYFHQGNNQTLDNILVSRHLLDHYEFDMVHVNSPFMEAHGRASDHDPLLLQLSF
SKENDKAESSKQSVKAKKTSKGKLLPKTGDSLVYVITLLGTASLLVPILLLTKGKKES
SEQ ID NO: 32 - SPy0843; gas158
MKKHLKTVALTLTTVSVVTHNQEVFSLVKEPILKQTQASSSISGADYAESSGKSKLKINETSGPVDDTVTD
LFSDKRTTPEKIKDNLAKGPREQELKAVTENTESEKQITSGSQLEQSKESLSLNKTVPSTSNWEICDFITK
GNTLVGLSKSGVEKLSQTDHLVLPSQAADGTQLIQVASFAFTPDKKTAIAEYTSRAGENGEISQLDVDGKE
IINEGEVFNSYLLKKVTIPTGYKHIGQDAFVDNKNIAEVNLPESLETISDYAFAHLALKQIDLPDNLKAIG
ELAFFDNQITGKLSLPRQLMRLAERAFKSNHIKTIEFRGNSLKVIGEASFQDNDLSQLMLPDGLEKIESEA
FTGNPGDDHYNNRVVLWTKSGKNPSGLATENTYVNPDKSLWQESPEIDYTKWLEEDFTYQKNSVTGFSNKG
LQKVKRNKNLEIPKQHNGVTITEIGDNAFRNVDFQNKTLRKYDLEEVKLPSTIRKIGAFAFQSNNLKSFEA
SDDLEEIKEGAFMNNRIETLELKDKLVTIGDAAFHINHIYAIVLPESVQEIGRSAFRQNGANNLIFMGSKV
KTLGEMAFLSNRLEHLDLSEQKQLTEIPVQAFSDNALKEVLLPASLKTIREEAFKKNHLKQLEVASALSHI
AFNALDDNDGDEQFDNKVVVKTHHNSYALADGEHFIVDPDKLSSTIVDLEKILKLIEGLDYSTLRQTTQTQ
FRDMTTAGKALLSKSNLRQGEKQKFLQEAQFFLGRVDLDKAIAKAEKALVTKKATKNGQLLERSINKAVLA
YNNSAIKKANVKRLEKELDLLTGLVEGKGPLAQATMVQGVYLLKTPLPLPEYYIGLNVYFDKSGKLIYALD
MSDTIGEGQKDAYGNPILNVDEDNEGYHALAVATLADYEGLDIKTILNSKLSQLTSIRQVPTAAYHRAGIF
QAIQNAAAEAEQLLPKPGTHSEKSSSSESANSKDRGLQSNPKTNRGRHSAILPRTGSKGSFVYGILGYTSV
ALLSLITAIKKKKY
SEQ ID NO: 33 - SPy1326; gas165
MVDLGESLYPERYDVTKSKAYIDLCHSYGAKRLFMSLLQLAPADHQMFHCYAELIAYANQLGIRVIADVSP
SFISQAGWSDQLIERAHAFGLAGLRLDEALPLAEIVTLTRNPFGLKIELNMSTDKQLLMSLLATDAERSNI
IGCHNFYPHEFTGLSWQHFKDMSRFYHEHDIETAAFITAQSASEGPWLLAEGLPTVEDHRHLPIGLQVELM
KAIGTIDNILISNQFISEEELAACTQALARPVTTIKVRPIIDLTEVEEQIIGYPHCYRGDVSDYVIRSTMP
RLVYAQESIAPRDQSKEVKRGSIIIDNDRYHRYKGELQIALKNFTVSSKANVVAEVREDYLSLLDDLRPWQ
EFCLETAPS
SEQ ID NO: 34 - SPy1972; gas187
MKKKVNQGSKRYQYLLKKWGIGFVIAATGTVVLGCTPSILTHQVAAKTIVGLARDEAQQGDGNAKSGDGLQ
SSSKEAKPVLDSSSANPASIAEHHLRMHFKTLPAGESLGSLGLWVWGDVDQPSKDWPNGAITMTKAKKDDY
GYYLDVPLAAKHRQQVSYLINNKAGENLSKDQHISLLTPKMNEVWIDENYHAHAYRPLKKGYLRINYHNQS
GHYDNLAVWTFKDVKTPTTDWPNGLDLSHKGHYGAYVDVPLKEGANEIGFLILDKSKTGDAIKVQPKDYLF
KELDNHTQVFVKDTDPKVYNNPYYIDQVSLKGAEQTTPNEIKAIFTTLDGLDEDAVKQNIKITDKAGKTVA
IDELTLDRDKSVMTLKGDFKAQGAVYTVTFGEVSQVARQSWQLKDKLYAYDGELGATLAKDGSVDLALWSP
SADTVKVVVYDKQDQTRVVGQADLTKSDKGVWRAHLTSDSVKGISDYTGYYYLYEITRGQEKVMVLDPYAK
SLAAWNDATATDDIKTAKAAFIDPSKLGPTGLDFAKINNFKKREDAIIYEAHVRDFTSDKALEGKLTHPFG
TFSAFVEQLDYLKDLGVTHVQLLPVLSYFYANELDKSRSTAYTSSDNNYNWGYDPQHYFALSGMYSANPND
PALRIAELKNLVNEIHKRGMGVIFDVVYNHTARTYLFEDLEPNYYHFMNADGTARESFGGGRLGTTHAMSR
RILVDSITYLTREFKVDGFRFDMMGDHDAAAIEQAFKAAKAINPNTIMIGEGWRTYQGDEGKKEIAADQDW
MKATNTVGVFSDDIRNTLKSGFPNEGTAAFITGGAKNLEGLFKTIKAQPGNFEADAPGDVVQYIAAHDNLT
LHDVIAKSINKDPKVAEEEIHKRIRLGNTMILTAQGTAFIHSGQEYGRTKQLLNPDYKTKASDDKVPNKAT
LIDAVAQYPYFIHDSYDSSDAVNHFDWAKATDSIAHPISNQTKAYTQGLIALRRSTDAFTKATKAEVDRDV
TLITQAGQDGIQQEDLIMGYQTVASNGDRYAVFVNADNKTRKVVLPQAYRYLLGAQVLVDAEQAGVTAIAK
PKGVQFTKEGLTIEGLTALVLKVSSKTANPSQQKSQTDNHQTKTPDGSKDLDKSLMTRPKRAKTNQKLPKT
GEASSKGLLAAGIALLLLAISLLMKRQKD
SEQ ID NO: 35 - SPy1983; gas188
MLTSKHHNLNKLVWRYGLTSAAAVLLAFGGGASSVKAEVSSTTMTSSQRESKIKEIEESLKKYPEVSNEKF
WERKWYGTYFKEEDFQKELKDFTEKRLKEILDLIGKSGIKGDRGETGPAGPAGPQGKTGERGAQGPKGDRG
EQGIQGKAGEKGERGEKGDKGETGERGEKGEAGIQGPQGEAGKDGAPGKDGAPGEKGEKGDRGETGAQGPV
GPQGEKGETGAQGPAGPQGEAGKPGEQGPAGPQGEAGQPGEKAPEKSPEGEAGQPGEKAPEKSKEVTPAAE
KPADKEANQTPERRNGNMAKTPVANNHRRLPATGEQANPFFTAAAVAVMTTAGVLAVTKRKENN
SEQ ID NO: 36 - SPy2009; gas190
MRRAENNKHSRYSIRKLSVGVTSIAIASLFLGKVAYAVDGIPPISLTQKTTATTSENWHHIDKDGLIPLGI
SLEAAKEEFKKEVEESRLSEAQKETYKQKIKTAPDKDKLLFTYHSEYMTAVKDLPASTESTTQPVEAPVQE
TQASASDSMVTGDSTSVTTDSPEETPSSESPVAPALSEAPAQPAESEEPSVAASSEETPSPSTPAAPETPE
EPAAPSPSPESEEPSVAAPSEETPSPETPEEPAAPSQPAESEESSVAATTSPSPSTPAESETQTPPAVTKD
SDKPSSAAEKPAASSLVSEQTVQQPTSKRSSDKKEEQEQSYSPNRSLSRQVRAHESGKYLPSTGEKAQPLF
IATMTLMSLFGSLLVTKRQKETKK
SEQ ID NO: 37 - SPy2010; gas191
MRKKQKLPFDKLAIALMSTSILLNAQSDIKANTVTEDTPATEQAVETPQPTAVSEEAPSSKETKTPQTPDD
AEETIADDANDLAPQAPAKTADTPATSKATIRDLNDPSQVKTLQEKAGKGAGTVVAVIDAGFDKNHEAWRL
TDKTKARYQSKEDLEKAKKEHGITYGEWVNDKVAYYHDYSKDGKTAVDQEHGTHVSGILSGNAPSETKEPY
RLEGAMPEAQLLLMRVEIVNGLADYARNYAQAIIDAVNLGAKVINMSFGNAALAYANLPDETKKAFDYAKS
KGVSIVTSAGNDSSFGGKTRLPLADHPDYGVVGTPAAADSTLTVASYSPDKQLTETATVKTADQQDKEMPV
LSTNRFEPNKAYDYAYANRGMKEDDFKDVKGKIALIERGDIDFKDKIANAKKAGAVGVLIYDNQDKGFPIE
LPNVDQMPAAFISRKDGLLLKENPQKTITFNATPKVLPTASGTKLSRFSSWGLTADGNIKPDIAAPGQDIL
SSVANNKYAKLSGTSMSAPLVAGIMGLLQKQYETQYPDMTPSERLDLAKKVLMSSATALYDEDEKAYFSPR
QQGAGAVDAKKASAATMYVTDKDNTSSKVHLNNVSDKFEVTVTVHNKSDKPQELYYQATVQTDKVDGKLFA
LAPKALYETSWQKITIPANSSKQVTIPIDVSQFSKDLLAPMKNGYFLEGFVRFKQDPTKEELMSIPYIGFR
GDFGNLSALEKPIYDSKDGSSYYHEANSDAKDQLDGDGLQFYALKNNFTALTTESNPWTIIKAVKEGVENI
EDIESSEITETIFAGTFAKQDDDSHYYIHRHANGKPYAAISPNGDGNRDYVQFQGTFLRNAKNLVAEVLDK
EGNVVWTSEVTEQVVKNYNNDLASTLGSTRFEKTRWDGKDKDGKVVANGTYTYRVRYTPISSGAKEQHTDF
DVIVDNTTPEVATSATFSTEDRRLTLASKPKTSQPVYRERIAYTYMDEDLPTTEYISPNEDGTFTLPEEAE
TMEGATVPLKMSDFTYVVEDMAGNITYTPVTKLLEGHSNKPEQDGSDQAPDKKPETKPEQDGSGQAPDKKP
ETKPEQDGSGQTPDKKPETKPEQDGSGQTPDKKPETKPEKDSSGQTPGKTPQKGQPSRTLEKRSSKRALAT
KASTKDQLPTTNDKDTNRLHLLKLVMTTFFLGLVAHIFKTKRTED
SEQ ID NO: 38 - SPy2018; gas192
MAKNNTNRHYSLRKLKTGTASVAVALTVLGAGFANQTEVKANGDGNPREVIEDLAANNPAIQNIRLRYENK
DLKARLENAMEVAGRDFKRAEELEKAKQALEDQRKDLETKLKELQQDYDLAKESTSWDRQRLEKELEEKKE
ALELAIDQASRDYHRATALEKELEEKKKALELAIDQASQDYNRANVLEKELETITREQEINRNLLGNAKLE
LDQLSSEKEQLTIEKAKLEEEKQISDASRQSLRRDLDASREAKKQVEKDLANLTAELDKVKEDKQISDASR
QGLRRDLDASREAKKQVEKDLANLTAELDKVKEEKQISDASRQGLRRDLDASREAKKQVEKALEEANSKLA
ALEKLNKELEESKKLTEKEKAELQAKLEAEAKALKEQLAKQAEELAKLRAGKASDSQTPDTKPGNKAVPGK
GQAPQAGTKPNQNKAPMKETKRQLPSTGETANPFFTAAALTVMATAGVAAVVKRKEEN
SEQ ID NO: 39 - SPy2025; gas193
MKKRKLLAVTLLSTILLNSAVPLVVADTSLRNSTSSTDQPTTADTDTDDESETPKKDKKSKETASQHDTQK
DHKPSHTHPTPPSNDTKQTDQASSEATDKPNKDKNDTKQPDSSDQSTPSPKDQSSQKESQNKDGRPTPSPD
QQKDQTPDKTPEKSADKTPEKGPEKATDKTPEPNRDAPKPIQPPLAAAPVFIPWRESDKDLSKLKPSSRSS
AAYVRHWTGDSAYTHNLLSRRYGITAEQLDGFLNSLGIHYDKERLNGKRLLEWEKLTGLDVRAIVAIAMAE
SSLGTQGVAKEKGANMFGYGAFDFNPNNAKKYSDEVAIRHMVEDTIIANKNQTFERQDLKAKKWSLGQLDT
LIDGGVYFTDTSGSGQRRADIMTKLDQWIDDHGSTPEIPEHLKITSGTQFSEVPVGYKRSQPQNVLTYKSE
TYSFGQCTWYAYNRVKELGYQVDRYMGNGGDWQRKPGFVTTHKPKVGYVVSFAPGQAGADATYGHVAVVEQ
IKEDGSILISESNVMGLGTISYRTFTAEQASLLTYVVGDKLPRP
SEQ ID NO: 40 - SPy2043; gas195
MNLLGSRRVFSKKCRLVKFSMVALVSATMAVTTVTLENTALARQTQVSNDVVLNDGASKYLNEALAWTFND
SPNYYKTLGTSQITPALFPKAGDILYSKLDELGRTRTARGTLTYANVEGSYGVRQSFGKNQNPAGWTGNPN
HVKYKIEWLNGLSYVGDFWNRSHLIADSLGGDALRVNAVTGTRTQNVGGRDQKGGMRYTEQRAQEWLEANR
DGYLYYEAAPIYNADELIPRAVVVSMQSSDNTINEKVLVYNTANGYTINYHNGTPTQK
SEQ ID NO: 41 - SPy0453; gas205
MGKRMSLILGAFLSVFLLVACSSTGTKTAKSDKLKVVATNSIIADMTKAIAGDKIDLHSIVPIGQDPHEYE
PLPEDVEKTSNADVIFYNGINLEDGGQAWFTKLVKNAQKTKNKDYFAVSDGIDVIYLEGASEKGKEDPHAW
LNLENGIIYSKNIAKQLIAKDPKNKETYEKNLKAYVAKLEKLDKEAKSKFDAIAENKKLIVTSEGCFKYFS
KAYGVPSAYIWEINTEEEGTPDQISSLIEKLKVIKPSALFVESSVDRRPMETVSKDSGIPIYSEIFTDSIA
KKGKPGDSYYAMMKWNLDKISEGLAK
SEQ ID NO: 42 - SPy0857; gas208
MTKKKGKLVLISLFVLAACLGAYSAMRQSHKTSNVSAETIASSSTRHFIDEIGPTASTIGQERDLYASVMI
AQAILESSNGKSSLSQAPYYNFFGIKGAYNGSSVTMSTWEDDGNGNTYTIDQAFRAYPSIADSLNDYADLL
SSSTYIGARKSNTLSYQDATAALTGLYATDTSYNLKLNNIIATYGLTAYDVANSSAQETGLATSGYVWNEY
RRNYTDAETLAVDEAWAKRMTY
SEQ ID NO: 43 - SPy1007; gas219
MSSVGVINLRNLYSTYDPTEVKGKINEGPPFSGSLFYKNIPYGNSSIELKVELNSVEKANFFSGKRVDIFT
LEYSPPCNSNIKKNSYGGITLSDGNRIDKKNIPVNIFIDGVQQKYSYTDISTGSTDKKEVTIQELDVKSRY
YLQKHFNIYGFGDVKDFGRSSRFQSGFEEGNIIFHLNSGERISYNLFDTGHGDRESMLKKYSDNKTAYSDQ
LHIDIYLVKFNK
SEQ ID NO: 44 - SPy1306; gas242
MSWNWKKTSVLGTLSLASVLPLTACVSGGGKGVKETDGKTIVVSVDEGYVDYIKSIKGEFEKEHKVTVKVK
KEGMMDTLDKLSTDGPTGASPDVFLAPFDRVGGLGTEGQIAEVTLGNSKEFDDTVKKLVTIDGKTYGAPDV
IETLVTYYNKDLVPQAPKSFTELEVLQKDSKFAFASEPGKSVGFLAKWTDFYYGYGLIAGYGGYIFGDKGT
KPSDLGLGNDGTVEGLNYAKQWYGTWPQGMQDTKKAGDFITEQFISKKAGVIIDGPWAASSFKDAGVNFGV
MEIPTLTNGKKYQPFAGGKAWVISNYSKGKTTAQKFLDYVTNAENQKRFYDKTQEIPANLTARNYASKEGN
ELTKAVISQFESAQPMPNIPEMAEVWEPGANMFFNVASGKEEASKAAKEAAKTIKEAIEQKYAE
SEQ ID NO: 45 - SPy1939; gas277
MTTMQKTISLLSLALLIGLLGTSGKAISVYAQDQHTDNVIAESTISQVSVEASMRGTEPYIDATVTTDQPV
RQPTQATITLKDASDNTINSWVYTMAAQQRRETAWFDLTGQKSGDYHVTVTVHTQEKAVTGQSGTVHFDQN
KARKTPTNMQQKDTSKAMTNSVDVDTKAQTNQSANQEIDSTSNPFRSATNHRSTSLKRSTKNEKLTPTASN
SQKNGSNKTKMLVDKEEVKPTSKRGFPWVLLGLVVSLAAGLFTATQKVSRRK
SEQ ID NO: 46 - SPy1813; gas380
MDKHLLVKRTLGCVCAATLMGAALATHHDSLNTVKAEEKTVQVQKGLPSIDSLHYLSENSKKEFKEELSKA
GQESQKVKEILAKAQQADKQAQELAKMKIPEKIPMKPLHGSLYGGYFRTWHDKTSDPTEKDKVNSMGELPK
EVDLAFIFHDWTKDYSLFWKELATKHVPKLNKQGTRVIRTIPWRFLAGGDNSGIAEDTSKYPNTPEGNKAL
AKAIVDEYVYKYNLDGLDVDVEHDSIPKVDKKEDTAGVERSIQVFEEIGKLIGPKGVDKSRLFIMDSTYMA
DKNPLIERGAPYINLLLVQVYGSQGEKGGWEPVSNRPEKTMEERWQGYSKyIRPEQyMIGFSFYEENAQEG
NLWYDINSRKDEDKANGINTDITGTRAERYARWQPKTGGVKGGIFSYAIDRDGVAHQPKKYAKQKEFKDAT
DNIFHSDYSVSKALKTVMLKDKSYDLIDEKDFPDKALREAVMAQVGTRKGDLERENGTLRLDNPAIQSLEG
LNKFKKLAQLDLIGLSRITKLDRSVLPANMKPGKDTLETVLETYKKDNKEEPATIPPVSLKVSGLTGLKEL
DLSGFDRETLAGLDAATLTSLEKVDISGNKLDLAPGTENRQIFDTMLSTISNHVGSNEQTVKFDKQKPTGH
YPDTYGKTSLRLPVANEKVDLQSQLLFGTVTNQGTLINSEADYKAYQNHKIAGRSFVDSNYHYNNFKVSYE
NYTVKVTDSTLGTTTDKTLATDKEETYKVDFFSPADKTKAVHTAKVIVGDEKTMMVNLAEGATVIGGSADP
VNARKVFDGQLGSETDNISLGWDSKQSIIFKLKEDGLIKHWRFFNDSARNPETTNKPIQEASLQIFNIKDY
NLDNLLENPNKFDDEKYWITVDTYSAQGERATAFSNTLNNITSKYWRVVFDTKGDRYSSPVVPELQILGYP
LPNADTIMKTVTTAKELSQQKDKFSQKMLDELKIKEMALETSLNSKIFDVTAINANAGVLKDCIEKRQLLK
K
SEQ ID NO: 47 - SPy0793; gas473
MIKDTFLKTNWLNISHHIILLVEGFYFSFYSLAKELVSSTAQPVNYYAHLLNVSFVGYIISLIGLSYYLSR
QVSRQLFLKTSFIVISYLIVSYWVQITQHLNDKRFDIWSLTKNQFYQFQALPSLLIILVMATLIKILAAYF
AIEKDRFGLLGYQGNTFSVALILAVVPINDIHLLKLISSRFSELVTAGNSQIALLKISGLLIVLLVIFATI
IYVVLNALKHLKSNKPSFSVAATTSLFLALVFNYTFQYGVKGDEALLGYYVFPGATLFQIVAITLVALLAY
VITNRYWPTTFFLLILGTIISVVNDLKESMRSEPLLVTDFVWLQELGLVTSFVKKSVIVEMVVGLAICIVV
AWYLHGRVLAGKLFMSPVKRASAVLGLFIVSCSMLIPFSYEKEGKILSGLPIISALNNDNDINWLGFSTNA
RYKSLAYVWTRQVTKKIMEKPTNYSQETIASIAQKYQKLAEDINKDRKNNIADQTVIYLLSESLSDPDRVS
NVTVSHDVLPNIKAIKNSTTAGLMQSDSYGGGTANMEFQTLTSLPFYNFSSSVSVLYSEVFPKMAKPHTIS
EFYQGKNRIAMHPASANNFNRKTVYSNLGFSKFLALSGSKDKFKNIENVGLLTSDKTVYNNILSLINPSES
QFFSVITMQNHIPWSSDYPEEIVAEGKNFTEEENHNLTSYARLLSFTDKETRAFLEKLTQINKPITVVFYG
DHLPGLYPDSAFNKHIENKYLTDYFIWSNGTNEKKNHPLINSSDFTAALFEHTDSKVSPYYALLTEVLNKA
SVDKSPDSPEVKAIQNDLKNIQYDVTIGKGYLLKHKTFFKISR
SEQ ID NO: 48 - SPy0838; gas477
MENWKFALSSIWGHKMRSILTMLGIIIGVAAVVIIMGLGNAMKNSVTSTFSSKQKDIQLYFQEKGEEEDLY
AGLHTHENNHEVKPEWLEQIVKDIDGIDSYYFTNSATSTISYEKKKVDNASIIGVSKDYFNIKNYDIVAGR
TLTDNDYSNFSRIILLDTVLADDLFGKGNYKSALNKVVSLSDKDYLVIGVYKTDQTPVSFDGLSGGAVMAN
TQVASEFGTKEIGSIYIHVNDIQNSMNLGNQAADMLTNISHIKDGQYAVPDNSKIVEEINSQFSIMTTVIG
SIAAISLLVGGIGVMNIMLVSVTERTREIGLRKALGATRLKILSQFLIESVVLTVLGGLIGLLLAQLSVGA
LGNAMTLKGACISLDVALIAVLFSASIGVFEGMLPANKASKLDPIEALRYE
SEQ ID NO: 49 - gi126660
MAKNTTNRHYSLRKLKTGTASVAVALTVVGAGLVAGQTVRADHSDLVAEKQRLEDLGQKFERLKQRSELYL
QQYYDNKSNGYKGDWYVQQLKMLNRDLEQAYNELSGEAHKDALGKLGIDNADLKAKITELEKSVEEKNDVL
SQIKKELEEAEKDIQFGREVHAADLLRHKQEIAEKENVISKLNGELQPLKQKVDETDRNLQQEKQKVLSLE
QQLAVTKENAKKDFELAALGHQLADKEYNAKIAELESKLADAKKDFELAALGHQHAHNEYQAKLAEKDGQI
KQLEEQKQILDASRKGTARDLEAVRQAKKATEAELNNLKAELAKVTEQKQILDASRKGTARDLEAVRKSKK
QQVEAALKQLEEQNKISEASRKGLRRDLDTSREAKKQVEKDLANLTAELDKVKEEKQISDASRQGLRRDLD
ASREAKKQVEKALEEANSKLAALEKLNKDLEESKKLTEKEKAELQAKLEAEAKALKEQLAKQAEELAKLRA
GKASDSQTPDAKPGNKAVpGKGQAPQAGTKPNQNKAPMKETKRQLPSTGETANPFFTAAALTVMAAA
SEQ ID NO: 50 - gi4586375
MAKNNTNRHYSLRKLKTGTASVAVALTVLGTGLASQTEVKADGEARDVVPELVANNLGLLRKRVARLQAEL
KTKEEKLRKLDLALGKEHIDNIALKHQLETEKREAEAQRQILENEKKKLEEELANKNTTLDGALRAITEKE
EKLRELDLALGKEHIDNIDLKHQLETEKREAEAQRQILENEKKKLEEELANKNTTLDGALRAITEKEEKLR
ELDLALGKEHIDNIDLKHQLETEKREAEAQRQILENEKKKLEEQNKISEASRKGLRRDLDASREAKKQLEA
EHQKLEEQNKISEASRKGLRRDLDASREAKKQVEKDLANLTAELDKVKEEKQISDASRQGLRRDLDASREA
KKQVEKALEEANSKLAALEKLNKELEESKKLTEKEKAELQAKLEAEAKALKEQLAKQAEELAKLRAGKASG
SQTPDAKPGNKAVPGKGQAPQAGAKPNQNKAPMKETKRQLPSTGETANPFLTAAALTVMATAGVAAVVKRK
EEN
SEQ ID NO: 51 - MGAS10270_SPy1784
MARKDTNKQYSLRKLKTGTASVAVAVAVLGAGFANQTTVKANSKNPVPVKKEAKLSEAELHDKIKNLEEEK
AELFEKLDKVEEEHKKVEEEHKKDHEKLEKKSEDVERHYLRQLDQEYKEQQERQKNLEELERQSQREVEKR
YQEQLQKQQQLEKEKQISEASRKSLSRDLEASRAAKKDLEAEHQKLKEEKQISEASRQGLSRDLEASREAK
KKVEADLAEANSKLQALEKLNKELEEGKKLSEKEKAELQAKLEAEAKALKEQLAKQAEELAKLKGNQTPNA
KVAPQANRSRSAMTQQKRTLPSTGETANPFFTAAAATVMVSAGMLALKRKEEN
SEQ ID NO: 52 - SPyM3_1727
MAKNNTNRHYSLRKLKTGTASVAVALTVLGTGLVAGQTVKADARSVNGEFPREVKLKNEIENLLDQVTQLY
TKHNSNYQQYNAQAGRLDLRQKAEYLKGLNDWAERLLQELNGEDVKKVLGKVAFEKDDLEKEVKELKEKID
KKEKEYQDLDKDFDLAKQGYVLSDKRHQQELEEKEKKVTEATAKVGQISEELETVKQKVESTMQDLTEKQN
RVSQLEQELATTKQNAKEDFELAALANAADKQKLEAKIADLETKLKEAKEDFELAALGHQHAHNEYQAKLA
EKDDQIKQLEEQKQILDASRKGTARDLEAVRQAKKATEAELNNLKAELAKVTEQKQILDASRKGTARDLEA
VRQAKAQVEAALKQLEEQNRISEASRKGLRRDLDASREAKKQVEKDLANLTAELDKVKEEKQISDASRQGL
RRDLDASREAKKQVEKALEEANSKLAALEKLNKELEESKKLTEKEKAELQAKLEAEAKALKEQLAKQAEEL
AKLRAGKASDSQIPDTKPGNKAVPGKGQAPQAGTKPNQNKAPMKETKRQLPSTGETANPFFTAAALTVMAT
AGVAAVVKRKEEN
SEQ ID NO: 53 - gi507127
MARKDTNKQYSLRKLKTGTASVAVAVAVLGAGFANQTEVKAEGVKKAEEAKLSVPKTEYDKLYDDYDKLQE
KSAEYLERIGELEERQKNLEKLERQSQVAADKHYQEQVKKHQEYKQEQEERQKNLEELERQNKREIDKRYK
EQLHKQQQLETEKQISEASRKSLSRDLEASREAKKKVEADLAALEAEHQKLKEEKQISDASRQSLSRDLEA
SREAKKKVEADLAALTAEHQKLKEEKQISDASRQGLSRDLEASREAKKKVEADLAEANSKLQALEKLNKEL
EEGKKLSEKEKAELQAKLEAEAKALKEQLAKQAEELAKLKGNQTPNAKVAPQANRSRSAMTQQKRTLPSTG
ETANPFFTAAAATVMVSAGMLALKRKEEN

1. A method of identifying S. pyogenes (GAS) infection as the cause of a neuropsychiatric or behavioural disorder in a patient, or of identifying a patient at risk of developing a neuropsychiatric or behavioural disorder caused by GAS infection, said method comprising the steps of:
a) contacting a biological sample from a patient suffering from a neuropsychiatric or behavioural disorder with at least one GAS antigen, wherein the at least one GAS antigen is selected from the group consisting of the amino acid sequences SEQ ID NO:41, SEQ ID NO:45, SEQ ID NO:23, SEQ ID NO:19, and SEQ ID NO:44 and fragments thereof that retain the ability to bind to antibodies that bind to the full-length GAS antigens under conditions appropriate for binding of any antibodies present in the biological sample to the at least one GAS antigen; and
b) detecting the presence of any antibodies in the biological sample bound to the at least one GAS antigen thereof, wherein the detection of antibodies against the at least one GAS antigen is indicative that the patient is suffering from a neuropsychiatric or behavioural disorder caused by GAS infection or that the patient is at risk of developing a neuropsychiatric or behavioural disorder caused by GAS infection.

2. The method of claim 1, wherein the neuropsychiatric or behavioural disorder is a tic disorder.

3. The method of claim 1, wherein step a) comprises contacting the sample with 1, 2, 3, 4, or 5 of the GAS antigens.

4. The method of claim 1, further comprising contacting the sample with one or more further GAS antigens, wherein the one or more further GAS antigens are selected from the group consisting of the amino acid sequences SEQ ID NOS:31, 2, 17, 3, 16, 47, 18, 5, 27, 21, 29, 25, 35, 9, 8, 13, 28, 33, 30, and 48 and fragments thereof that retain the ability to bind to antibodies that bind to the full-length GAS antigens under conditions appropriate for binding of any antibodies present in the biological sample to the one or more GAS antigens and detecting the presence of antibodies in the biological sample bound to the one or more further GAS antigens.

5. The method of claim 1 further comprising contacting the sample with one or more further GAS antigens, wherein the one or more further GAS antigens are selected from the group consisting of the amino acid sequences SEQ ID NOS:6, 53, 37, 51, 24, 46, 26, 20, 49, 4, 32, 42, 50, 39, 52, 10, 40, 38, 1, 34, 14, 36, 22, 12, and 43 and fragments thereof that retain the ability to bind to antibodies that bind to the full-length GAS antigens, under conditions appropriate for binding of any antibodies present in the biological sample to the one or more GAS antigens and detecting the presence of antibodies in the biological sample bound to the one or more further GAS antigens.

6. The method of claim 1, wherein the biological sample is a saliva sample, a blood sample or a serum sample.

7. The method of claim 6, wherein the biological sample is a serum sample.

8. The method of claim 1, wherein the biological sample is from an adolescent or from a child.

9. The method of claim 1, wherein the biological sample is from a patient displaying clinical symptoms of pharyngitis.

10. The method of claim 1, wherein the GAS antigens are displayed on one or more protein arrays.

11. A protein array comprising at least two S. pyogenes (GAS) antigens having an amino acid sequence selected from the group consisting of SEQ ID NO:41, SEQ ID NO:45. SEQ ID NO:23, SEQ ID NO:19, and SEQ ID NO:44 and fragments thereof that retain the ability to bind to antibodies that bind to the full-length GAS antigens.

12. An array according to claim 11 wherein the array comprises at least one additional GAS antigen, wherein the at least one additional GAS antigen is selected from the group consisting of the amino acid sequences SEQ ID NOS:31, 2, 17, 3, 16, 47, 18, 5, 27, 21, 29, 25, 35, 9, 8, 13, 28, 33, 30, and 48 and fragments thereof that retain the ability to bind to antibodies that bind to the full-length GAS antigens.

13. An array according to claim 11, wherein the array comprises at least one additional GAS antigen, wherein the at least one additional GAS antigen is selected from the group consisting of the amino acid sequences SEQ ID NOS:6, 53, 37, 51, 24, 46, 26, 20, 49, 4, 32, 42, 50, 39, 50, 10, 40, 38, 1, 34, 14, 36, 22, 12, and 43 and fragments thereof that retain the ability to bind to antibodies that bind to the full-length GAS antigens.
